(19) United States
(12) Patent Application Publication
(10) Pub. No.: US 2025/0114455 A1
(43) Pub. Date: Apr. 10, 2025

(54) RADIOSENSITIZING COMPOSITIONS AND METHODS OF USE THEREOF

(71) Applicant: University of Georgia Research Foundation, Inc., Athens, GA (US)

(72) Inventors: Jin Xie, Athens, GA (US); Ian Michael Delahunty, Athens, GA (US)

(21) Appl. No.: 18/833,322

(22) PCT Filed: Jan. 30, 2023
(86) PCT No.: PCT/US2023/061583

§ 371 Date: Jul. 25, 2024

Related U.S. Application Data
(60) Provisional application No. 63/304,403, filed on Jan. 28, 2022.

(51) Int. Cl. A61K 41/00; A61K 47/54; A61N 5/10; C07J 9/00

(52) U.S. Cl. CPC A61K 41/0038; A61K 47/554; A61N 5/10; C07J 9/00; A61N 2005/1087; A61N 2005/109; A61N 2005/1098

(57) ABSTRACT

Compositions and methods of improving radiation therapy are provided. The composition typically includes an effective amount of 7-DHC or a derivative or analog thereof. The methods typically include administering the composition to a subject in need thereof in combination with one or more doses of radiation therapy, preferably non-ultraviolet radiation therapy. A preferred radiation is ionizing radiation. The compositions and methods can be used to treat tumors, e.g., benign and malignant tumors. Thus, methods of treating cancer are provided. In preferred embodiments, the 7-DHC or a derivative or analog thereof is encapsulated or incorporated into nanoparticles. The particles can be, for example, polymeric nanoparticles, lipoprotein or lipoprotein-like particles, liposomes, inorganic nanoparticles, or a combination thereof. In some embodiments, the 7-DHC or analog thereof is derivatized with a polymer or saccharide, which is some embodiments, can self-assemble into particles. In some embodiments, the nanoparticles further include a targeting agent coupled thereto.

### CROSS-REFERENCE TO RELATED APPLICATIONS

This application claims the benefit and priority to U.S. Ser. No. 63/304,403 filed Jan. 28, 2022, and which is incorporated by reference herein in its entirety.

### STATEMENT REGARDING FEDERALLY SPONSORED RESEARCH OR DEVELOPMENT

This invention was made with government support under R01EB022596 and R01CA247769 awarded by the NIH. The government has certain rights in the invention.

### REFERENCE TO THE SEQUENCE LISTING

The Sequence Listing submitted as a text file named “UGA_2020-065-02_PCT_ST26.xml” created on Jan. 16, 2023, and having a size of 8,288 bytes is hereby incorporated by reference pursuant to 37 C.F.R. § 1.834(c)(1).

### FIELD OF THE INVENTION

The field of the invention generally relates to compositions for increasing the effectiveness of radiation therapy and use thereof for treatment of ailments such as cancer.

### BACKGROUND OF THE INVENTION

Radiotherapy remains a mainstream treatment option for cancer, with nearly 50% patients receiving it at least once during the treatment course (Delaney, et al., Nat. Rev. Cancer, 11, 239-253 (2011)). Despite technological advances such as stereotactic body radiotherapy, intensity-modulated radiation therapy, and image-guided radiation therapy, the efficacy of radiotherapy is limited by normal tissue toxicities (McKelvey, et al., Front. Oncol., 9, 1504 (2019), Burbach, et al., Radiother Oncol., 113, 1-9 (2014)). To improve treatment outcomes, chemotherapeutics, such as cisplatin, fluorouracil, and docetaxel, can be administered during radiotherapy, i.e., chemo-radiotherapy (Gustaysson, et al., Clin. Colorectal Cancer, 14, 1-10 (2015)). However, chemo-radiotherapy is associated with elevated systemic toxicities, which frequently delay or interrupt chemotherapy or radiotherapy (Engstrom, et al., J. Natl. Compr. Cancer Netw., 7, 778-831 (2009)). It is desired that a prodrug can be activated inside tumors by external beam radiation, thereby enhancing the efficacy of radiation therapy without causing side effects to normal tissues. Such techniques, however, are not currently available in the clinic.

Thus, it is an object of the invention to provide improved compositions and methods of use thereof for sensitizing cancer to radiotherapy.

### SUMMARY OF THE INVENTION

It has been discovered that dehydrocholesterol (7-DHC) can serve as a sensitizer for radiation. Thus, compositions and methods of improving radiation therapy are provided. The compositions, e.g., a pharmaceutical composition, typically includes an effective amount of 7-DHC or a derivative or analog thereof. Exemplary analogs and derivatives of 7-DHC are provided, see, e.g., Formulae I-III. Typically, these derivatives and analogs are radiosensitizers, that operate through the same or similar mechanism as 7-DHC, see, e.g., Scheme 1. In some embodiments, the 7-DHC analog is 8-DHC or a derivative or analog according to Formulae IV-VI that can be converted into 7-DHC or a derivative or analog thereof by an isomerase in vivo. For example, in some embodiments, the 7-DHC or a derivative or analog thereof begins (e.g., is administered) as a compound of Formulae IV, V, or VI, and is converted to a corresponding compound of Formulae I, II, or III, respectively, by an isomerase in vivo. Thus, in some embodiments, the compound of Formulae IV, V, or VI is a prodrug. Thus, the compounds are preferably a radiosensitizer or prodrug thereof.

In some embodiments, the compounds, particularly those of Formulae III and VI includes a therapeutic agent, for example a small molecule chemotherapeutic agent, coupled thereto.

The methods typically include administering the composition to a subject in need thereof in combination with one or more doses of radiation therapy, preferably non-ultraviolet radiation therapy. A preferred radiation is ionizing radiation. The radiation may include x-rays, gamma rays, neutrons, protons, or a combination thereof. The compositions and methods can be used to treat tumors, e.g., benign and malignant tumors. Thus, the compositions and methods can be used to treat cancer.

In preferred embodiments, the 7-DHC or derivative or analog thereof is encapsulated or incorporated into or form part of nanoparticles. The particles can be, for example, polymeric nanoparticles, lipoprotein or lipoprotein-like particles, liposomes, inorganic nanoparticles, or a combination thereof. The polymeric nanoparticles can be composed of one or more amphiphilic, hydrophobic, and/or hydrophilic polymers. Exemplary hydrophobic polymers include, but are not limited to, polyesters such as poly(lactic acid-co-glycolic acid)s, poly(lactic acid), poly(glycolic acid). In a particular embodiment, the polymeric nanoparticles include or consist of poly(lactic acid-co-glycolic acid) (PLGA). In some embodiments, 7-DHC or an analog thereof is derivatized with a hydrophilic polymer or a hydrophilic saccharide; or is a salt thereof. In such embodiments, the derivatized compounds self-assembles into nanoparticles alone, or in further combination with other elements, e.g., other polymers or saccharides.

The nanoparticles can be lipoprotein or lipoprotein-like nanoparticles, e.g., low-density lipoprotein (LDL) or LDL-like nanoparticles. For example, in some embodiments, the nanoparticles include one or more triglyceride(s) and one or more phospholipid(s). Exemplary triglycerides include oleic acid (OA) and/or triolein (TO). Exemplary phospholipids include phosphatidyl choline (PC). The lipoprotein or lipoprotein-like nanoparticles can be synthetic or isolated from a natural source, e.g., human subject, optionally wherein the lipoprotein is natural LDL. In some embodiments, the lipoprotein-like particle is free from protein, optionally with the exception of a polypeptide-based targeting moiety.

Typically, the nanoparticles have a size suitable for delivery of the 7-DHC or derivative or analog thereof to tumor microenvironments by enhanced permeability and retention. In some embodiments, the nanoparticles have a size of about 10 nm to about 300 nm, or about 50 nm to about 150 nm.

In some embodiments, the nanoparticles further include a targeting agent coupled thereto. In particular embodiments, the targeting agent targets NTSR1, e.g., is an agonist or antagonist for NTSR1. Exemplary agents that target NTSR1 are NTS and variants thereof, NTSₘᵤₜ. SR142948A, and NTS_{20.8}. See also SEQ ID NOS:1-7.

In preferred methods, the combination of 7-DHC or derivative or analog thereof and radiation enhances the treatment of the cancer compared to administration of the radiation alone. In some embodiments, the same dose of radiation is more effective than when administered in the absence of the pharmaceutical composition, a lower dose of radiation has the same effectiveness as a higher dose when administered in the absence of the pharmaceutical composition, or a combination thereof.

The cancer can be a radiosensitive cancer, or a radioresistant cancer. The cancer can be a vascular, bone, muscle, bladder, brain, breast, cervical, cob-rectal, esophageal, kidney, liver, lung, nasopharyngeal, pancreatic, prostate, skin, stomach, uterine, or germ cell. In some embodiments, the cancer is an epithelial cancer. In some embodiments, the cancer is a non-small cell lung cancer (NSCLC).

In some embodiments, a dose of radiation is administered after administration of the pharmaceutical composition. For example, the dose of radiation can be administered 1 to 48 hours, or 1 to 24 hours, or 1 to 12 hours, or 1 to 6 hours, or 2 to 6 hours, or 1, 2, 3, 4, or 5 hours after administration of the pharmaceutical composition. In some embodiments, 1, 2, 3, 4, 5, 6, 7, 8, 9, 10, or more rounds of administration of the pharmaceutical composition is followed by administration of the dose of radiation, optionally wherein the dose of radiation is administered after one, some, or all of the rounds of administration of the pharmaceutical composition.

### BRIEF DESCRIPTION OF THE DRAWINGS

FIGS. 1A-1E illustrate physicochemical characterizations of 7-DHC@PLGA NPs. FIG. 1A is an image of negative staining TEM image of 7-DHC@PLGA NPs. Scale bar, 100 nm. FIG. 1B is a graph showing the zeta potential of 7-DHC@PLGA NPs in PBS and water. FIG. 1C is a bar graph showing DLS analysis of 7-DHC@PLGA NPs in water. FIGS. 1D and 1E are line graphs showing size change of 7-DHC@PLGA NPs in PBS under 37° C. incubation for 48 h (1D) and over 5 days (1E). FIG. 1F 7-DHC release from 7-DHC@PLGA NPs, tested in solutions with pH 7.4, 6.5, and 5.5, respectively.

FIGS. 2A-2E illustrate the impact of 7-DHC@PLGA NPs on cell lipid peroxidation and viability, tested in CT26 cells. FIG. 2A is a bar graph showing cell viability in the absence of radiation, measured by MTT assays at 24 h. Both 7-DHC@PLGA NPs and free 7-DHC showed minimal toxicity when 7-DHC concentration was below 12.5 μg/mL. FIG. 2B is a bar graph showing cell viability in the absence of radiation, measured by ATP bioluminescence at 24 h. FIGS. 2C and 2D are bar graphs showing the impact on cell lipid peroxidation, tested with 7-DHC@PLGA NPs (5 μg/mL concentration) in the presence of radiation (5 Gy). PBS, 7-DHC@PLGA NPs, and irradiation alone (IR) were tested as controls. FIG. 2C shows Caspase-3 activity, measured by FAM-FLICA Caspase 3/7 assay, and FIG. 2D shows cell viability, evaluated by MTT assays at 24 h. Carnosine, which inhibits the formation of alpha-beta unsaturated aldehydes, was added during treatment for comparison. FIG. 2E is a curve showing the results of a clonogenic assay, tested with PBS, irradiation (IR), and 7-DHC@PLGA NPs plus irradiation (7-DHC@PLGA NPs+IR). Survival fractions relative to the PBS control were fit into the linear quadratic (LQ) model. *, P<0.05; **, P<0.01; ***, P<0.001; ****, P<0.0001; ns, no significant difference.

FIGS. 3A-3H illustrate the impact of 7-DHC@PLGA NPs on mitochondria and intracellular ROS. FIG. 3A is a spectrogram of LC/MS data of 16:0-18:2 PC without (upper) and with (lower) 5 Gy irradiation in the presence of 7-DHC. FIG. 3B is bar graph showing BODIPY lipid peroxidation assay results. A decrease in the 590/510 nm fluoresce intensity ratio indicates an increased level of lipid peroxidation. FIG. 3C is a bar graph showing MDA levels, measured by TBARS assay. Cells treated with 7-DHC@PLGA NPs+IR showed a dramatic increase of MDA level. FIG. 3D is a bar graph showing Ψₘ changes, measured by JC-1 staining. A decrease in fluorescence intensity relative to PBS control indicates a drop in Ψₘ. DMSO and carbonyl cyanide m-chlorophenylhydrazone (CCCP) were tested as negative and positive controls, respectively. FIG. 3E is a bar graph showing MnSOD activity results. FIG. 3F is a bar graph showing DHE assay results. An increase in fluorescence indicates an elevated superoxide level in cells. FIG. 3G is a bar graph showing DNA double-strand breaks, measured by counting positively stained γH2AX foci using ImageJ. FIG. 3H is a bar graph showing Cytochrome c release, evaluated by anti-cytochrome c and mitochondrial complex Vα double staining. Colocalization rate was assessed by ImageJ. A decrease in colocalization rate indicates enhanced cytochrome c release. *, P<0.05; **, P<0.01; ***, P<0.001; ****, P<0.0001; ns, no significant difference.

FIGS. 4A-4H illustrate results from In vivo therapy studies. Live image study was conducted in CT26 tumor bearing nude mice. FIGS. 4A-4C are time-lapsed NIR fluorescence (0.5 min, 4 and 24 h post injection). FIG. 4D is a bar graph showing Radiant efficiency (p/sec/cm2/sr/μW/cm2) in different organ after 24 h post injection. FIG. 4E is a bar graph showing ROI analyses of in vivo radiotracer uptake (tumor-to-muscle ratio). Therapy experiments were conducted in CT26 tumor bearing nude mice and balb/c mice. FIG. 4F is a scheme showing the treatment schedule. 7-DHC@PLGA NPs were intravenously (i.v.) injected, followed by beam radiation (3 Gy) applied to tumors at 4 h (7-DHC@PLGA NPs+IR; n=5). A total of three doses of treatment was given two days apart. Irradiation only (IR), 7-DHC@PLGA NPs only (7-DHC@PLGA NPs), and carrier only (PBS) were tested (n=5). FIG. 4G is a tumor volume curve. 7-DHC@PLGA NPs+IR caused effective tumor regression, with 30% of the animals being tumor-free on Day 43. *, P<0.05; **, P<0.01; ***, P<0.001; ****, P<0.0001; ns, no significant difference. FIG. 4H is an animal survival curve. Sixty percent of the animals in the 7-DHC@PLGA NPs+IR group remained alive on Day 43, while all animals in control groups had either died or reached a humane endpoint.

FIGS. 5A and 5B report the result of experiments investigating potential side effects of 7-DHC@PLGA NPs. FIG. 5A is a line graph showing body weight change. No significant body weight drop was observed throughout the entire therapy study. FIG. 5B is a line graph serum calcium level change. 7-DHC@PLGA NPs (1 mg/kg), calcitriol (200 μg/kg), 7-DHC (200 μg/kg), and PBS were intraperitoneally injected into healthy balb/c mice (n=3). Blood samples were collected at 0, 2, 6, and 24 h for analysis. No calcium level increase was observed in animals injected with 7-DHC@PLGA NPs.

FIG. 6 is an illustration of the proposed mechanisms behind 7-DHC@PLGA-NPs-based radiosensitization. 7-DHC@PLGA NPs are accumulated in tumors through the EPR effect. 7-DHC is released from the NPs and accumulated in cancer cells' plasma and mitochondrial membrane. Under radiation, 7-DHC triggers and propagates radical chain reactions that cause lipid peroxidation and mitochondria damage, exacerbating oxidative stress in cells. Meanwhile, lipid peroxidation also produces toxic oxysterols and aldehydes that react with DNA and other biomolecules. All these events culminate at inducing cancer cell death.

FIGS. 7A-7D are bar graphs showing the size distribution of 7-DHC@LDL (FIGS. 7A and 7B) and cholesterol@LDL (FIGS. 7C and 7D) particles before (FIGS. 7A and 7C) and after (FIGS. 7B and 7D) extrusion. FIGS. 7E and 7F are bar graphs showing the zeta potential of 7-DHC@LDL (FIG. 7E) and cholesterol@LDL (FIG. 7F) particles in water, PBS, and Tris Buffer pH 8. FIGS. 7G and 7H are line graphs showing the drug release from 7-DHC@LDL (FIG. 7G) and cholesterol@LDL (FIG. 7H) particles at pH 5.5, 6.5, and 7.4 over 3 days. FIG. 7I is a line graph showing the change in 7-DHC@LDL and cholesterol@LDL particle diameter over 7 days. FIGS. 7J and 7K are bar graphs showing the change in 7-DHC@LDL (FIG. 7J) and cholesterol@LDL (FIG. 7K) zeta potential after 7 days. FIG. 7L is a flow chart illustrating a method of preparing LDL particles.

FIGS. 8A and 8B are bar graphs showing the hydrodynamic size distributions of 7-DHC@LDL-NTS (FIG. 8A) and cholesterol@LDL-NTS (FIG. 8B) NPs. FIG. 8C shows the zeta potential (mV) of 7-DHC@LDL-NTS and cholesterol@LDL-NTS NPs. FIG. 8D is a bar graphs showing cellular update of 7-DHC@LDL and cholesterol@LDL NPs with and without an NTS targeting moiety.

FIG. 9A is a bar graph showing the viability (%) of H-1299 cells as determined by an MTT assay following treatment of NTS-targeted or untargeted 7-DHC@LDL and cholesterol@LDL NPs with and without ionizing radiation (“IR”), compared to IR alone, and the inhibitor “In” aripiprazole with or without IR. FIG. 9B is a bar graph showing the level of γH2AX of 7-DHC@LDL-NTS NPs with and with IR. FIGS. 9C and 9D are bar graphs showing detection of reactive oxygen species (RO) in various treatment groups according to a Singlet Oxygen Sensor Green (SOSG) test (FIG. 9C) and as measured Superoxidase Dismutase (SOD) activity (U/ml) (FIG. 9D). FIGS. 9E and 9F are bar graphs illustrating lipid peroxidation as evidenced by changes in 4-hydroxynonenal (4-HNE) (FIG. 9E) and malondialdehyde (MDA) (FIG. 9F).

FIG. 10 are 24-hour images of the tumor targeting ability with exemplary 7-DHC nanoparticles, 7-DHC-encapsulated liposomes. H1299 tumor bearing mice were used. The nanoparticles were coupled with or without a tumor targeting ligand (NTS, which binds to NTSR1 or neurotensin receptor 1 that is overexpressed in many types of tumors) and were labeled with a near-infrared dye. Whole-body fluorescence images were taken on a small animal imaging scanner.

FIGS. 11A-11C are graphs showing the radiosensitizing effects of 7-DHC-liposomes on tumor volume (FIG. 11A), survival (FIG. 11B), and body weight (FIG. 11C) in H1299 tumor bearing mice. The nanoparticles were coupled with NTS for tumor targeting, and were intravenously (i.v.) administered at 2.3 mg 7 DHC/kg. Radiation (RT, 5 Gy) was applied to tumors 24 hours post-particle injection. The rest of the animal body was lead-shielded during radiation. A total of three treatments were given, two days apart.

### DETAILED DESCRIPTION OF THE INVENTION

### I. Definitions

The terms “inhibit” and “reduce” mean to reduce or decrease in activity or expression. This can be a complete inhibition or reduction of activity or expression, or a partial inhibition or reduction. Inhibition or reduction can be compared to a control or to a standard level. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

The term “nanoparticle” refers to any particle having a diameter greater than 1 nm and less than 1000 nm.

The term “targeting agent” refers to a chemical compound that can direct a nanoparticle to a receptor site on a selected cell or tissue type, can serve as an attachment molecule, or serve to couple or attach another molecule. The term “direct,” as relates to chemical compounds, refers to causing a nanoparticle to preferentially attach to a selected cell or tissue type. This targeting agent, generally binds to its receptor with high affinity and specificity.

The terms “treatment” and “treating”, as used herein, refer to the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

The term “radiosensitivity” refers to the relative susceptibility of cells to the harmful effect of ionizing radiation. The more radiosensitive a cell is, the less radiation that is required to kill that cell. In general, it has been found that cell radiosensitivity is directly proportional to the rate of cell division and inversely proportional to the cell's capacity for DNA repair.

The term “radioresistant” refers to a cell that does not die when exposed to clinically suitable dosages of ionizing radiation.

The term “neoplastic cell” refers to a cell undergoing abnormal cell proliferation (“neoplasia”). The growth of neoplastic cells exceeds and is not coordinated with that of the normal tissues around it. The growth typically persists in the same excessive manner even after cessation of the stimuli, and typically causes formation of a tumor.

The term “tumor” or “neoplasm” refers to an abnormal mass of tissue containing neoplastic cells. Neoplasms and tumors may be benign, premalignant, or malignant.

The term “cancer” or “malignant neoplasm” refers to a cell that displays uncontrolled growth, invasion upon adjacent tissues, and often metastasis to other locations of the body.

The term “antineoplastic” refers to a composition, such as a drug or biologic, that can inhibit or prevent cancer growth, invasion, and/or metastasis.

The term “individual,” “host,” “subject,” and “patient” are used interchangeably to refer to any individual who is the target of administration or treatment. The subject can be a vertebrate, for example, a mammal. Thus, the subject can be a human or veterinary patient.

The term “therapeutically effective” means that the amount of the composition used is of sufficient quantity to ameliorate one or more causes or symptoms of a disease or disorder. Such amelioration only requires a reduction or alteration, not necessarily elimination. A therapeutically effective amount of a composition for treating cancer is preferably an amount sufficient to cause tumor regression or to sensitize a tumor to radiation or chemotherapy.

The term “pharmaceutically acceptable” refers to a material that is not biologically or otherwise undesirable, i.e., the material may be administered to a subject without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +1-2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the embodiments and does not pose a limitation on the scope of the embodiments unless otherwise claimed. No language in the specification should be construed as indicating any non-claimed element as essential to the practice of the invention.

Unless otherwise indicated, the disclosure encompasses conventional techniques of molecular biology, microbiology, cell biology and recombinant DNA, which are within the skill of the art. Unless otherwise noted, technical terms are used according to conventional usage, and in the art, such as in the references cited herein, each of which is specifically incorporated by reference herein in its entirety.

### II. Compositions

Compositions and methods of using 7-dehydrocholesterol (7-DHC) and derivatives and analogs thereof are provided. The 7-DHC and derivative and analog compounds can be used as a radiosensitizer for cancer treatment. In some embodiments, the compounds are encapsulated by or form part of a nanoparticle composition. Preferably, the compounds and compositions increase cancer cell death when used in combination with ionizing radiation.

### A. Radiosensitizer Compounds

### 1. 7-Dehydrocholesterol

In some embodiments, the radiosensitizer compound is 7-dehydrocholesterol (7-DHC).

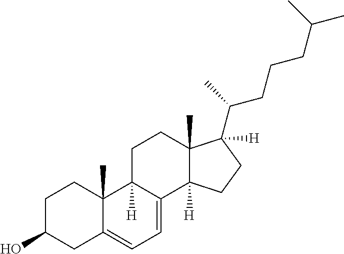

7-DHC is a biosynthetic precursor of cholesterol. Possessing a conjugated diene in the ring B, 7-DHC is an excellent hydrogen atom donor and highly susceptible to radical oxidation. In fact, 7-DHC affords the highest propagation rate towards free radical chain reaction among lipid molecules (2260 M⁻¹s⁻¹, compared to 11 M⁻¹s⁻¹ for cholesterol) (Babaei, et al., Clin. Colorectal Cancer, 17, e129-e142 (2018)). Normally, 7-DHC is converted by 7-dehydrocholesterol reductase (DHCR7) to cholesterol. When DHCR7 is deregulated or dysfunctional, however, 7-DHC accumulates in tissues, along with a reduced level of cholesterol. This disruption of cholesterol homeostasis causes extensive lipid peroxidation and tissue damage over time. This is evidenced in patients with Smith-Lemli-Opitz Syndrome (SLOS), who harbor mutations in their DHCR7 gene. SLOS patients often suffer developmental disorders including multiple congenital malformations and mental retardation because of the accumulation of 7-DHC in cholesterol-rich tissues, such as the brain (Babaei, et al., Clin. Colorectal Cancer, 17, e129-e142 (2018), Beppu, et al., J. Anus Rectum Colon, 1, 65-73 (2017)).

Additionally, vitamin D₃ is produced in the skin from 7-dehydrocholesterol by UV irradiation, which breaks the B ring to form pre-D₃ (Bikle, “Vitamin D: Production, Metabolism, and Mechanisms of Action,” [Updated 2017 Aug. 11]. In: Feingold K R, Anawalt B, Boyce A, et al., editors. Endotext [Internet]. South Dartmouth (MA): MDText.com, Inc.). Pre-D₃ isomerizes to D₃ or with continued UV irradiation to tachysterol and lumisterol. D₃ is preferentially removed from the skin, bound to vitamin D binding protein (DBP). The liver and other tissues metabolize vitamin D, whether from the skin or oral ingestion, to 25OHD, the principal circulating form of vitamin D. Several enzymes have 25-hydroxylase activity, but CYP2R1 is the most important. 25OHD is then further metabolized to 1.25(OH)₂D principally in the kidney, by the enzyme CYP27B 1, although other tissues including various epithelial cells, cells of the immune system, and the parathyroid gland contain this enzymatic activity. 1.25(OH)₂D is the principal hormonal form of vitamin D, responsible for most of its biologic actions.

Provided herein are compositions and methods of using 7-DHC as a radiosensitizing agent, inspired by its ability to promote radical chain oxidation. Results provided below show that 7-DHC can react with radiation-induced ROS, initiating and propagating radical chain reactions, thereby enhancing the efficacy of radiotherapy (Scheme 1, below). Because 7-DHC radicalization is governed by radiation, which applies conformly to tumors during radiotherapy, the radiosensitizing effects are believed to be highly tumor-specific. Unlike in SLOS patients, DHCR7 is functional in most cancer patients and can convert 7-DHC to non-toxic cholesterol outside tumors (Ma, et al., nanobiotechnology, 19, 1-10 (2021)). Hence, the risks for systemic and long-term toxicity are low.

### 2. Analogs and Derivatives of 7-DHC

a. Exemplary Analogs and Derivatives

In some instances, a derivative or analog of 7-DHC is used in the methods or pharmaceutical formulations described herein. Preferably the 7-DHC derivative or analog can react with radiation-induced ROS, and initiate and propagate radical chain reactions or release a coupled therapeutic agent, thereby enhancing the efficacy of radiotherapy in the same or similar manner as 7-DHC (e.g., according to similar chemical mechanism as illustrated in Scheme 1, below for 7-DHC). In preferred embodiments, the analogs and derivatives maintain a conjugated diene in the ring in the B or C ring of the sterol.

In some embodiments, such diene containing analogs and derivatives can be synthesized, for example, from precursors, such as 8-DHC in the presence of isomerase. In still other instances, an analog of 7-DHC is 8-DHC, or an analog or derivative thereof, which can be used in the methods or pharmaceutical formulations described herein. The 8-DHC or analog or derivative thereof can be converted into 7-DHC or derivative or analog thereof (e.g., having a conjugated diene in the ring B) by an isomerase, e.g., in vivo, for example in or around tumors. Tumors can express isomerases and may even have elevated expression of isomerases. See, e.g., Bao, et al., Am J Pathol, 164(5):1727-37 (2004). Doi: 10.1016/S0002-9440(10)63731-5; Chou, et al., Carcinogenesis, 40(3):461-473 (2019), doi: 10.1093/carcin/bgy155; Chen, et al., Oncology Reports, 38, 1822-1832 (2017). Doi: 10.3892/or.2017.5846; Obama, et al., Clin Cancer Res, (12) (1) 70-76 (2006), DOI: 10.1158/1078-0432.CCR-05-0588, each of which is specifically incorporated by reference herein in its entirety. Thus, in some embodiments, 8-DHC, or a derivative or analog thereof, is selected for treatment of an isomerase overexpressing cancer.

The analog or derivative typically is not cholesterol. Thus, in some embodiments, cholesterol is expressly excluded as the 7-DHC analog or derivative.

Non-limiting exemplary derivatives or analogs of 7-DHC can have a chemical structure according to Formula I:

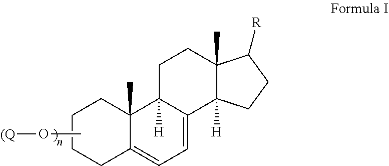

wherein n is an integer value selected from 1, 2, 3, or 4;
wherein R is a linear or branched alkyl or heteroalkyl group, alkenyl or heteroalkenyl, or alkynyl or heteroalkynyl group, where each may be optionally substituted by one or more substituents; and
wherein Q is a hydrogen, a linear or branched alkyl or heteroalkyl group, alkenyl or heteroalkenyl, or alkynyl or heteroalkynyl group, a cycloalkyl group, a heterocycloalkyl, an aryl, a heteroaryl group, acyl group, a sulfonate group, or a siloxy group, where each may be optionally substituted with one or more substituents;
or is a salt thereof.

In certain instances, n is 1 for the derivatives or analogs of Formula I and the position of the “Q-O-” is preferably at the 3-position of the A ring. However, the “Q-O—” group may be present at any of positions 1, 2, 3, or 4 of the A ring. In some cases, the “Q-O—” group is preferably located at the 2 and/or 3 position of the A ring. It is also contemplated that multiple “Q-O-” groups may be present, up to 4, when n is 4 and all of positions 1, 2, 3, or 4 of the A ring are substituted by the group. It is further understood that in instances of such multiple substitutions that the Q group of each substituent is independently selected from the selections for Q described above.

In some other instances, 7-DHC can be an amphiphilic analog having a chemical structure according to Formula II:

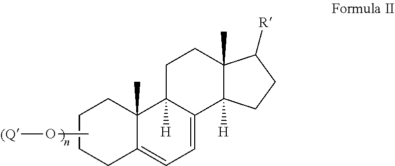

wherein n is an integer value selected from 1, 2, 3, or 4;
wherein R′ is a linear or branched alkyl or heteroalkyl group, alkenyl or heteroalkenyl, or alkynyl or heteroalkynyl group, where each may be optionally substituted by one or more substituents; and
wherein Q′ is a hydrophilic polymer or a hydrophilic saccharide; or is a salt thereof.

In some instances, the hydrophilic polymer is polyalkylene oxide, such as polypropylene glycol or polyethylene glycol (PEG). The chain length and molecular weights of the polyalkylene oxide are not particularly restricted. PEGs, for example, can be obtained in various lengths from commercial sources. In some non-limiting instances, the weight average molecular weight of the polyalkylene oxide can be between about 1 kDa and 1000 kDa, or any sub-range disclosed therein. In some instances, the hydrophilic polymer can be a polysaccharide, such as cellulose and starch and derivatives thereof; a hydrophilic polypeptide, such as poly-L-glutamic acid, gamma-polyglutamic acid, poly-L-aspartic acid, poly-L-serine, or poly-L-lysine; a poly(oxyethylated polyol); a poly(olefinic alcohol), such as poly(vinyl alcohol); poly(vinylpyrrolidone); a poly(N-hydroxyalkyl methacrylamide), such as poly(N-hydroxyethyl methacrylamide); a poly(N-hydroxyalkyl methacrylate), such as poly(N-hydroxyethyl methacrylate); a hydrophilic poly(hydroxy acids); polyacrylic acids, polyethylenimine, and copolymers thereof. In some instances, the saccharide is a monosaccharide or disaccharide. In some instances, the saccharide can be selected from glucose, cyclodextrin, galactose, mannose, melibiose, maltose, sucrose, glucuronic acid, galacturonic acid, mannuronic acid, diglucuronic acid, melibiouronic acid and maltouronic acid, hyaluronic acid.

In certain instances, n is 1 for the derivatives or analogs of Formula II and the position of the “Q′-O—” is preferably at the 3-position of the A ring. However, the “Q′-O—” group may be present at any of positions 1, 2, 3, or 4 of the A ring. In some cases, the “Q′-O—” group is preferably located at the 2 and/or 3 position of the A ring. It is also contemplated that multiple “Q′-O” groups may be present, up to 4, when n is 4 and all of positions 1, 2, 3, or 4 of the A ring are substituted by the group. It is further understood that in instances of such multiple substitutions that the Q′ group of each substituent is independently selected from the selections for Q′ described above.

In some embodiments, an amphiphilic analog of 7-DHC is able to self-assemble and can form into particles, such as nanoparticles, alone, or in combination with other elements such as polymers, lipids, and/or carbohydrates. The average size, such as diameter, of the particles are not particularly restricted. In some non-limiting instances, the average diameter of the particles can range from about 1 to 500 nm or 1 to 250 nm, or any subrange disclosed therein. Such (nano)particles, formed from the amphiphilic analog of 7-DHC may be used in the methods or pharmaceutical formulations described herein.

In some instances, there may be a radical responsive linker present between Q′ and O in Formula II. Alternatively, the radical responsive linker may replace —O— in the A ring of Formula II. In some instances, the radical responsive linker can be selected from a diselenide, thioketals, arylboronic esters, aminoacrylates, or peroxalate esters. Radical responsive linkers are known in the art (see Tao, et al., Asian J. of Pharm. Sci., Volume 13, Issue 2, March 2018, Pages 101-112; Liang, et al., Bioeng. Transl. Med. 2016, 1, 239-251; Xu, et al., Macromol. Biosci. 2016, 16, 635-46; Saravanakumar, et al., Adv Sci. 2017, 4, 1600124; and Ye, et al., Biomacromolecules. 2019, 20, 2441-2463, which are specifically incorporated by reference herein in their entireties.) It is believed that 7-DHC analogs or derivatives of Formula II can themselves function as a radiosensitizer for promoting lipid peroxidation. However, the enhancement may be more effective when 7-DHC is free rather than being part of a conjugate, as of Formula II. Accordingly, when a radical responsive linker is present between Q′ and the A ring then under irradiation it can cause the release 7-DHC.

Still other non-limiting exemplary derivatives or analogs of 7-DHC can have a chemical structure according to Formula III:

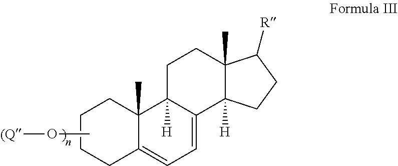

wherein n is an integer value selected from 1, 2, 3, or 4;
wherein R″ is a linear or branched alkyl or heteroalkyl group, alkenyl or heteroalkenyl, or alkynyl or heteroalkynyl group, where each may be optionally substituted by one or more substituents; and
wherein Q″ is a therapeutic agent;
or is a salt thereof.

In certain instances, n is 1 for the derivatives or analogs of Formula III and the position of the “Q″-O—” is preferably at the 3-position of the A ring. However, the “Q″-O—” group may be present at any of positions 1, 2, 3, or 4 of the A ring. In some cases, the “Q″-O—” group is preferably located at the 2 and/or 3 position of the A ring. It is also contemplated that multiple “Q″-O—” groups may be present, up to 4, when n is 4 and all of positions 1, 2, 3, or 4 of the A ring are substituted by the group. It is further understood that in instances of such multiple substitutions that the Q″ group of each substituent is independently selected from the selections for Q″ described above.

In some instances, there may be a radical responsive linker present between Q″ and O in Formula III. Alternatively, the radical responsive linker may replace —O— in the A ring of Formula II. In some instances, the radical responsive linker can be selected from a diselenide, thioketals, arylboronic esters, aminoacrylates, or peroxalate esters. Radical responsive linkers are known in the art (see publications listed earlier).

In some other instances, the 7-DHC analog or derivative is 8-DHC or an analog or derivative thereof. 8-DHC derivatives or analogs may be used having a chemical structure according to Formula IV:

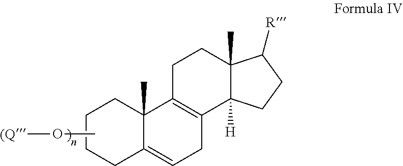

wherein n is an integer value selected from 1, 2, 3, or 4;
wherein R″′ is a linear or branched alkyl or heteroalkyl group, alkenyl or heteroalkenyl, or alkynyl or heteroalkynyl group, where each may be optionally substituted by one or more substituents; and
wherein Q″′ is a hydrogen, a linear or branched alkyl or heteroalkyl group, alkenyl or heteroalkenyl, or alkynyl or heteroalkynyl group, a cycloalkyl group, a heterocycloalkyl, an aryl, a heteroaryl group, acyl group, a sulfonate group, or a siloxy group, where each may be optionally substituted with one or more substituents;
or is a salt thereof.

It is believed that such 8-DHC derivatives or analogs of Formula IV will be isomerized into 7-DHC form, having a conjugated diene, by isomerases which are present, for example, in tumors.

In certain instances, n is 1 for the derivatives or analogs of Formula IV and the position of the “Q″′—O—” is preferably at the 3-position of the A ring. However, the “Q″′—O—” group may be present at any of positions 1, 2, 3, or 4 of the A ring. In some cases, the “Q″′—O—” group is preferably located at the 2 and/or 3 position of the A ring. It is also contemplated that multiple “Q″′—O—” groups may be present, up to 4, when n is 4 and all of positions 1, 2, 3, or 4 of the A ring are substituted by the group. It is further understood that in instances of such multiple substitutions that the Q″′ group of each substituent is independently selected from the selections for Q″′ described above.

In some other instances, 8-DHC analogs or derivatives are used which are amphiphilic and have a chemical structure according to Formula V:

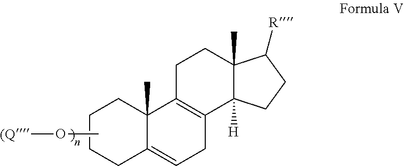

wherein n is an integer value selected from 1, 2, 3, or 4;
wherein R″″ is a linear or branched alkyl or heteroalkyl group, alkenyl or heteroalkenyl, or alkynyl or heteroalkynyl group, where each may be optionally substituted by one or more substituents; and
wherein Q″″ is a hydrophilic polymer or a hydrophilic saccharide; or is a salt thereof.

It is believed that such 8-DHC amphiphiles of Formula V will be isomerized into 7-DHC form, having a conjugated diene, by isomerases which are present, for example, in tumors.

In some instances, the hydrophilic polymer is polyalkylene oxide, such as polypropylene glycol or polyethylene glycol (PEG). The chain length and molecular weights of the polyalkylene oxide are not particularly restricted. PEGs, for example, can be obtained in various lengths from commercial sources. In some non-limiting instances, the weight average molecular weight of the polyalkylene oxide can be between about 1 kDa and 1000 kDa, or any sub-range disclosed therein. In some instances, the hydrophilic polymer can be a polysaccharide, such as cellulose and starch and derivatives thereof; a hydrophilic polypeptide, such as poly-L-glutamic acid, gamma-polyglutamic acid, poly-L-aspartic acid, poly-L-serine, or poly-L-lysine; a poly(oxyethylated polyol); a poly(olefinic alcohol), such as poly(vinyl alcohol); poly(vinylpyrrolidone); a poly(N-hydroxyalkyl methacrylamide), such as poly(N-hydroxyethyl methacrylamide); a poly(N-hydroxyalkyl methacrylate), such as poly(N-hydroxyethyl methacrylate); a hydrophilic poly(hydroxy acids); polyacrylic acids, polyethylenimine, and copolymers thereof. In some instances, the saccharide is a monosaccharide or disaccharide. In some instances, the saccharide can be selected from glucose, cyclodextrin, galactose, mannose, melibiose, maltose, sucrose, glucuronic acid, galacturonic acid, mannuronic acid, diglucuronic acid, melibiouronic acid and maltouronic acid, hyaluronic acid.

In certain instances, n is 1 for the derivatives or analogs of Formula V and the position of the “Q″″—O—” is preferably at the 3-position of the A ring. However, the “Q″″—O—” group may be present at any of positions 1, 2, 3, or 4 of the A ring. In some cases, the “Q″″—O—” group is preferably located at the 2 and/or 3 position of the A ring. It is also contemplated that multiple “Q″″—O—” groups may be present, up to 4, when n is 4 and all of positions 1, 2, 3, or 4 of the A ring are substituted by the group. It is further understood that in instances of such multiple substitutions that the Q″″ group of each substituent is independently selected from the selections for Q″″ described above.

In some embodiments, an amphiphilic 8-DHC of formula V is able to self-assemble and can form into particles, such as nanoparticles, alone, or in combination with other elements such as polymers, lipids, and/or carbohydrates. The average size, such as diameter, of the particles are not particularly restricted. In some non-limiting instances, the average diameter of the particles can range from about 1 nm to 500 nm or 1 nm to 250 nm, or any subrange disclosed therein. Such (nano)particles, formed from the amphiphilic 8-DHC may be used in the methods or pharmaceutical formulations described herein.

In some instances, there may be a radical responsive linker present between Q″″ and O in Formula V. Alternatively, the radical responsive linker may replace —O— in the A ring of Formula V. In some instances, the radical responsive linker can be selected from a diselenide, thioketals, arylboronic esters, aminoacrylates, or peroxalate esters. Radical responsive linkers are known in the art (see publications listed earlier).

Still other non-limiting exemplary 8-DHC derivatives or analogs can have a chemical structure according to Formula VI:

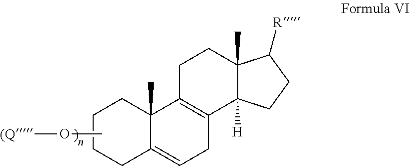

wherein n is an integer value selected from 1, 2, 3, or 4;
wherein R″″′ is a linear or branched alkyl or heteroalkyl group, alkenyl or heteroalkenyl, or alkynyl or heteroalkynyl group, where each may be optionally substituted by one or more substituents; and
wherein Q″″′ is a therapeutic agent;
or is a salt thereof.

It is believed that such 8-DHC derivatives or analogs of Formula VI will be isomerized into 7-DHC form, having a conjugated diene, by isomerases which are present, for example, in tumors.

In certain instances, n is 1 for the derivatives or analogs of Formula VI and the position of the “Q″″′-O—” is preferably at the 3-position of the A ring. However, the “Q″″′-O—” group may be present at any of positions 1, 2, 3, or 4 of the A ring. In some cases, the “Q″″′-O—” group is preferably located at the 2 and/or 3 position of the A ring. It is also contemplated that multiple “Q″″′-O—” groups may be present, up to 4, when n is 4 and all of positions 1, 2, 3, or 4 of the A ring are substituted by the group. It is further understood that in instances of such multiple substitutions that the Q″″′ group of each substituent is independently selected from the selections for Q″″′ described above.

In some instances, there may be a radical responsive linker present between Q″″′ and O in Formula VI. Alternatively, the radical responsive linker may replace —O— in the A ring of Formula II. In some instances, the radical responsive linker can be selected from a diselenide, thioketals, arylboronic esters, aminoacrylates, or peroxalate esters. Radical responsive linkers are known in the art (see publications listed earlier).

In some instances of Formulae I-VI, R, R′, R″, R″′, R″″. and R″″″ may have the structure shown below:

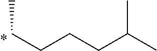

Method of synthesizing and purifying compounds according to Formulae I-VI are well-known to those skilled in the art. For example, hydrophilic polymer or therapeutic agents can be coupled to the A ring by reaction between a hydroxyl on the A ring and a suitable reactive group present on a hydrophilic polymer or therapeutic agent using coupling chemistry known to the person of ordinary skill in the art. Further, the skilled person understands that compounds encompassed by Formulae I-VI described above may have one or more chiral centers and thus exist as one or more stereoisomers. Such stereoisomers can exist as a single enantiomer, a mixture of diastereomers or a racemic mixture and are encompassed by the present disclosure. As used herein, the term “stereoisomers” refers to compounds made up of the same atoms having the same bond order but having different three-dimensional arrangements of atoms which are not interchangeable. The three-dimensional structures are called configurations. As used herein, the term “enantiomers” refers to two stereoisomers which are non-superimposable mirror images of one another. As used herein, the term “optical isomer” is equivalent to the term “enantiomer”. As used herein the term “diastereomer” refers to two stereoisomers which are not mirror images but also not superimposable. The terms “racemate”, “racemic mixture” or “racemic modification” refer to a mixture of equal parts of enantiomers. The term “chiral center” refers to a carbon atom to which four different groups are attached. Choice of the appropriate chiral column, eluent, and conditions necessary to effect separation of the pair of enantiomers is well known to one of ordinary skill in the art using standard techniques (see e.g. Jacques, J. et al., “Enantiomers, Racemates, and Resolutions”, John Wiley and Sons, Inc. 1981).

As used above, the term “alkyl” is given its ordinary meaning in the art and refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some cases, the alkyl group may be a lower alkyl group, i.e., an alkyl group having 1 to 10 carbon atoms (e.g., methyl, ethyl, propyl, butyl, pentyl, hexyl, heptyl, octyl, nonyl, or decyl). In some embodiments, a linear chain or branched chain alkyl may have 30 or fewer carbon atoms in its backbone, and, in some cases, 20 or fewer. In some embodiments, a straight chain or branched chain alkyl may have 12 or fewer carbon atoms in its backbone (e.g., C₁-C₁₂ for straight chain, C₃-C₁₂ for branched chain), 6 or fewer, or 4 or fewer. Likewise, cycloalkyls may have from 3-10 carbon atoms in their ring structure, or 5, 6 or 7 carbons in the ring structure. Examples of alkyl groups include, but are not limited to, methyl, ethyl, propyl, isopropyl, cyclopropyl, butyl, isobutyl, t-butyl, cyclobutyl, hexyl, and cyclohexyl. The terms “alkenyl” and “alkynyl” are given their ordinary meaning in the art and refer to unsaturated aliphatic groups analogous in length and possible substitution to the alkyls described, but that contain at least one double or triple bond, respectively. Illustrative alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl and the like.

The term “heteroalkyl” is given its ordinary meaning in the art and refers to an alkyl group, typically having one to ten carbons, in which one or more carbon atoms is replaced by a heteroatom. Suitable heteroatoms include oxygen, sulfur, nitrogen, phosphorus, and the like. Examples of heteroalkyl groups can include, but are not limited to, alkoxy, alkoxyalkyl, thioester, poly(ethylene glycol), and alkyl-substituted amino groups. The terms “heteroalkenyl” and “heteroalkynyl” are given their ordinary meaning in the art and refer to unsaturated aliphatic groups analogous in length and possible substitution to the heteroalkyls described, but that contain at least one double or triple bond, respectively.

The term “aryl” is given its ordinary meaning in the art and refers to aromatic carbocyclic groups, optionally substituted, having a single ring (e.g., phenyl), multiple rings (e.g., biphenyl), or multiple fused rings in which at least one is aromatic (e.g., 1,2,3,4-tetrahydronaphthyl, naphthyl, anthryl, or phenanthryl). That is, at least one ring may have a conjugated pi electron system, while other, adjoining rings can be cycloalkyls, cycloalkenyls, cycloalkynyls, aryls and/or heterocyclyls. The aryl group may be optionally substituted, as described herein. The term “heteroaryl” is given its ordinary meaning in the art and refers to aryl groups comprising at least one heteroatom as a ring atom. A “heteroaryl” is a stable heterocyclic or polyheterocyclic unsaturated moiety having preferably 3-14 carbon atoms, each of which may be substituted or unsubstituted. In some cases, a heteroaryl is a cyclic aromatic radical having from five to ten ring atoms of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like. It will also be appreciated that aryl and heteroaryl moieties, as defined above may be attached via an alkyl or heteroalkyl moiety and thus also include -(alkyl)aryl, -(heteroalkyl)aryl, -(heteroalkyl)heteroaryl, and -(heteroalkyl)heteroaryl moieties. Thus, as used herein, the phrases “aryl or heteroaryl” and “aryl, heteroaryl, -(alkyl)aryl, -(heteroalkyl)aryl, -(heteroalkyl)heteroaryl, and -(heteroalkyl)heteroaryl” are interchangeable. These may be optionally substituted.

It will further be appreciated that the above groups and/or compounds, as described herein, may be optionally substituted with any number of substituents or functional moieties. That is, any of the above groups may be optionally substituted. As used herein, the term “substituted” is contemplated to include all permissible substituents of organic compounds, “permissible” being in the context of the chemical rules of valence known to those of ordinary skill in the art. In general, the term “substituted” whether preceded by the term “optionally” or not, and substituents contained in the formulas above, refer to the replacement of hydrogen radicals in a given structure with the radical of a specified substituent. When more than one position in any given structure may be substituted with more than one substituent selected from a specified group, the substituent may be either the same or different at every position. It will be understood that “substituted” also includes that the substitution results in a stable compound, e.g., which does not spontaneously undergo transformation such as by rearrangement, cyclization, elimination, etc. In some cases, “substituted” may generally refer to replacement of a hydrogen with a substituent as described herein. However, “substituted,” as used herein, does not encompass replacement and/or alteration of a key functional group by which a molecule is identified, e.g., such that the “substituted” functional group becomes, through substitution, a different functional group. For example, a “substituted phenyl group” must still comprise the phenyl moiety and cannot be modified by substitution, in this definition, to become, e.g., a pyridine ring. In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents can be one or more and the same or different for appropriate organic compounds. The heteroatoms such as nitrogen may have hydrogen substituents and/or any permissible substituents of organic compounds described herein which satisfy the valences of the heteroatoms. The term “stable,” as used herein, preferably refers to compounds which possess stability sufficient to allow manufacture and which maintain the integrity of the compound for a sufficient period of time to be detected and preferably for a sufficient period of time to be useful for the purposes detailed herein.

Non-limiting examples of substituents include, but are not limited to, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, aryl, aryloxy, perhaloalkoxy, aralkoxy, heteroaryl, heteroaryloxy, heteroarylalkyl, heteroaralkoxy, azido, amino, halide, alkylthio, oxo, acylalkyl, carboxy esters, -carboxamido, acyloxy, aminoalkyl, alkylaminoaryl, alkylaryl, alkylaminoalkyl, alkoxyaryl, arylamino, aralkylamino, alkylsulfonyl, -carboxamidoalkylaryl, -carboxamidoaryl, hydroxyalkyl, haloalkyl, alkylaminoalkylcarboxy-, aminocarboxamidoalkyl-, cyano, alkoxyalkyl, perhaloalkyl, arylalkyloxyalkyl, and the like. The term “halogen” as used herein refer to an atom selected from the group consisting of fluorine, chlorine, bromine, and iodine.

b. Therapeutic Agents

As introduced above, in some embodiments, 7-DHC and 8-DHC analogs or derivatives, particularly those of Formulae III and VI, can be coupled to a therapeutic agent, preferably which can be release in vivo, e.g., by cleavage of a radical responsive linker, when present. Such therapeutic agents can be, for example, peptides, carbohydrates, polysaccharides, nucleic acid molecules, and organic molecules, as well as diagnostic agents (e.g., fluorescent compounds, etc.). In some embodiments, the therapeutic agent is a small molecule drug.

Therapeutic agents include antibiotics, antivirals, anti-parasites (helminths, protozoans), anti-cancer (e.g., chemotherapeutic agents), peptide drugs, anti-inflammatories, nutraceuticals such as vitamins, and oligonucleotide drugs (including DNA, RNAs including mRNAs, antisense, siRNA, miRNA, anti-miRNA, piRNA, aptamers, ribozymes, external guide sequences for ribonuclease P, and triplex forming agents such as tcPNAs).

In some embodiments, the therapeutic agent is a chemotherapeutic and/or antineoplastic drug. The majority of chemotherapeutic drugs can be divided into alkylating agents, antimetabolites, anthracyclines, plant alkaloids, topoisomerase inhibitors, monoclonal antibodies, and other antitumour agents.

Non-limiting examples of antineoplastic drugs that damage DNA or inhibit DNA repair include carboplatin, carmustine, chlorambucil, cisplatin, cyclophosphamide, dacarbazine, daunorubicin, doxorubicin, epirubicin, idarubicin, ifosfamide, lomustine, mechlorethamine, mitoxantrone, oxaliplatin, procarbazine, temozolomide, and valrubicin.

In some embodiments, the antineoplastic drug is a histone deacetylase inhibitor, which suppresses DNA repair at the transcriptional level and disrupt chromatin structure. In some embodiments, the antineoplastic drug is a proteasome inhibitor, which suppresses DNA repair by disruption of ubiquitin metabolism in the cell. Ubiquitin is a signaling molecule that regulates DNA repair. In some embodiments, the antineoplastic drug is a kinase inhibitor, which suppresses DNA repair by altering DNA damage response signaling pathways.

Additional antineoplastic drugs include, but are not limited to, alkylating agents (such as temozolomide, cisplatin, carboplatin, oxaliplatin, mechlorethamine, cyclophosphamide, chlorambucil, dacarbazine, lomustine, carmustine, procarbazine, chlorambucil and ifosfamide), antimetabolites (such as fluorouracil, gemcitabine, methotrexate, cytosine arabinoside, fludarabine, and floxuridine), some antimitotics, and vinca alkaloids such as vincristine, vinblastine, vinorelbine, and vindesine), anthracyclines (including doxorubicin, daunorubicin, valrubicin, idarubicin, and epirubicin, as well as actinomycins such as actinomycin D), cytotoxic antibiotics (including mitomycin, plicamycin, and bleomycin), and topoisomerase inhibitors (including camptothecins such as irinotecan and topotecan and derivatives of epipodophyllotoxins such as amsacrine, etoposide, etoposide phosphate, and teniposide) and cytoskeletal targeting drugs such as paclitaxel.

In some embodiments the therapeutic agent is a second radiosensitizer. Examples of known radiosensitizers include cisplatin, gemcitabine, 5-fluorouracil, pentoxifylline, vinorelbine, PARP inhibitors, histone deacetylase inhibitors, and proteasome inhibitors.

### B. Nanoparticles

Free 7-DHC is poorly soluble in water (Bai, et al., nanobiotechnology, 18, 1-10 (2020)). In some embodiments, the 7-DHC or derivative or analog thereof is encapsulated or incorporated into or otherwise forms part of nanoparticles, such as polymeric nanoparticles, lipoprotein-like particles, liposomes, inorganic nanoparticles, or a combination thereof. In some embodiments, the particles have a targeting moiety conjugated or otherwise linked thereto.

As introduced above, in some embodiments, 7-DHC or analog thereof is derivatized with a polymer or saccharide that facilitates incorporation into particles, and optionally can self-assemble into particles. In some embodiments, the 7-DHC or analog thereof does not include a polymer or saccharide. Such embodiments by include, for example, incorporation of the compound into particles, e.g., substituted for cholesterol or a cholesterol ester in LDL-like particles, and more traditional loading of the compound into polymeric particles.

The size of the particles can vary. In some forms the particle size is between about 5 nm and less than 1,000 nm. In some forms, the particle size is between about 10 nm and about 750 nm. In some forms, the particle size is between about 10 nm and about 500 nm. In some forms, the particle size is between about 10 nm and about 250 nm. In some forms, the particle size is between about 50 nm and about 250 nm. In some forms, the particle size is between about 50 nm and about 150 nm. In some forms, the particle size is between about 75 nm and about 125 nm. In some forms, the particle size is about 10 nm, about 20 nm, about 30 nm, about 40 nm, about 50 nm, about 60 nm, about 70 nm, about 80 nm, about 90 nm, about 100 nm, about 110 nm, about 120 nm, about 130 nm, about 140 nm, or about 150 nm.

### 1. Particle Composition

a. Polymeric Nanoparticles

In some forms, the nanoparticles can be a matrix of biocompatible polymers, preferably biodegradable polymers. The polymers can be amphiphilic, hydrophobic, or hydrophilic polymers that can be broken down hydrolytically or enzymatically in vitro or in vivo. Exemplary polymers are discussed below. Copolymers such as random, block, or graft copolymers, or blends of the polymers listed below can also be used.

The weight average molecular weight can vary for a given polymer but is generally between about 1000 Daltons and 1,000,000 Daltons, between about 1000 Daltons and about 500,000 Dalton, between about 1000 Daltons and about 250,000 Daltons, between about 1000 Daltons and about 100,000 Daltons, between about 5,000 Daltons and about 100,000 Daltons, between about 5,000 Daltons and about 75,000 Daltons, between about 5,000 Daltons and about 50,000 Daltons, or between about 5,000 Daltons and about 25,000 Daltons.

The extracellular microenvironment of tumor tissue is generally more mildly acidic than healthy tissue. In addition, the lumen of endosomes and lysosomes are also generally more acidic than the cytoplasm of a cell. Accordingly, to enhance the release of the cargo from nanoparticles in a tumor site prior to or post-nanoparticle uptake via the process of polymer degradation, diffusion, or both, the polymers can be acidic pH-responsive. In these forms, the polymers can contain ionizable groups (e.g. amine group(s)) that can become ionized and cause the nanoparticles to swell, or the polymers can contain a chemical moiety (such as disulfides, orthoesters, acetals, ketals, hydrazones, imines, cis-aconityls, esters, vinyl ethers, etc.) that can be cleaved more rapidly in an environment having acidic pH (such between 6.9 and 4.0) compared to an environment with a higher pH (such as 7.2, 8, 9, or higher).

i. Amphiphilic Polymers

The NPs can contain one or more amphiphilic polymers, preferably biodegradable amphiphilic polymers. The amphiphilic polymers contain a hydrophobic polymer portion and a hydrophilic polymer portion. The hydrophobic polymer portion and hydrophilic polymer portion can include any of the hydrophobic polymers and hydrophilic polymers, respectively, described in the corresponding titular sections below. In a non-limiting example, the hydrophobic polymer portion is a polymer formed from a polyester such as polyhydroxy acids (such as poly(lactic acid), poly(glycolic acid), and poly(lactic acid-co-glycolic acid)s), polycaprolactones, polyhydroxyalkanoates (such as poly-3-hydroxybutyrate, poly4-hydroxybutyrate, polyhydroxyvalerates), poly(lactide-co-caprolactones); poly(anhydride)s; poly(orthoester)s; hydrophobic polysaccharides (such as acetylated dextran, acetylated dextran, acetylated cellulose, proprionylated dextran, proprionylated cellulose); and hydrophobic polyethers (such as polypropylene glycol); as well as copolymers thereof. The hydrophilic polymer portion can contain a polymer such as a polyalkylene oxide such as polypropylene glycol or polyethylene glycol (PEG); polysaccharides such as cellulose and starch; hydrophilic polypeptides such as poly-L-glutamic acid, gamma-polyglutamic acid, poly-L-aspartic acid, poly-L-serine, or poly-L-lysine; poly(oxyethylated polyol); poly(olefinic alcohol) such as poly(vinyl alcohol); poly(vinylpyrrolidone); polyacrylamides or polymethaacrylamides including poly(N-hydroxyalkyl methacrylamides) such as poly(N-hydroxyethyl methacrylamide); poly(N-hydroxyalkyl methacrylates) such as poly(N-hydroxyethyl methacrylate); hydrophilic poly(hydroxy acids); and copolymers thereof. Examples of amphiphilic polymers that can be generated from this group include polyester-PEG copolymers such as poly(lactic acid-co-glycolic acid)-PEG (PLGA-PEG), poly(lactic acid)-PEG (PLA-PEG), poly(glycolic acid)-PEG (PGA-PEG), and polycaprolactone-PEG (PCL-PEG); hydrophobic polyethers-PEG, such as polypropylene glycol-PEG (PPG-PEG), PEG-PPG-PEG, PPG-PEG-PPG; and acetylated dextran-PEG. In some forms, the amphiphilic polymer can be PLGA-PEG.

ii. Hydrophobic Polymers

The NPs can be formed of one or more hydrophobic polymers. In some forms, the hydrophobic polymers are biodegradable. Examples of suitable hydrophobic polymers include polyesters such as polyhydroxy acids (such as poly(lactic acid-co-glycolic acid)s, poly(lactic acid), poly(glycolic acid)), polycaprolactones, polyhydroxyalkanoates (such as poly-3-hydroxybutyrate, poly4-hydroxybutyrate, polyhydroxyvalerates), poly(lactide-co-caprolactones); poly(anhydride)s; poly(orthoester)s; hydrophobic polysaccharides (such as acetylated dextran, acetylated dextran, acetylated cellulose, proprionylated dextran, proprionylated cellulose); as well as copolymers thereof.

In some forms, the hydrophobic polymers include polyesters such as polyhydroxy acids (such as poly(lactic acid-co-glycolic acid)s, poly(lactic acid), poly(glycolic acid)), polycaprolactones, polyhydroxyalkanoates (such as poly-3-hydroxybutyrate, poly4-hydroxybutyrate, polyhydroxyvalerates), poly(lactide-co-caprolactones); poly(anhydride)s; poly(orthoester)s; poly(beta-amino ester)s; and copolymers thereof.

iii. Hydrophilic Polymers

The NPs can contain one or more hydrophilic polymers. Preferably, the hydrophilic polymers are biodegradable. Hydrophilic polymers include polyalkylene glycol such as polyethylene glycol (PEG); polysaccharides such as cellulose and starch and derivatives thereof; hydrophilic polypeptides such as poly-L-glutamic acid, gamma-polyglutamic acid, poly-L-aspartic acid, poly-L-serine, or poly-L-lysine; poly(oxyethylated polyol); poly(olefinic alcohol) such as poly(vinyl alcohol); poly(vinylpyrrolidone); poly(N-hydroxyalkyl methacrylamide) such as poly(N-hydroxyethyl methacrylamide); poly(N-hydroxyalkyl methacrylate) such as poly(N-hydroxyethyl methacrylate); hydrophilic poly(hydroxy acids); and copolymers thereof. In some forms, the hydrophilic polymer is a polyalkylene glycol such as PEG or a poloxamer.

b. Lipoprotein and LDL-Like Particles

Over the last decade, use of lipoprotein based nanoparticles to deliver diagnostic and/or therapeutic agents to treat cancer has been investigated (Hill et al., Acad Radiol, 17 (2010) 1359-1365; Marotta et al., Nanomedicine-Uk, 6 (2011) 475-487; Murakami et al., Nanomedicine (Lond), 5 (2010) 867-879; Shazad et al., Neoplasia, 13 (2011) 309-319; Skajaa et al., Biomaterials, 32 (2011) 206-213; Zhou et al., J Control Release, 148 (2010) 380-387; Corbin et al., Nanomedicine (Lond), (2012)). These carriers are attractive vehicles for oncology applications due to their nanoscale size, fine particle size distribution, high payload carrying capacity and ability to avoid MPS surveillance (Gotto et al., Methods Enzymol, 128 (1986) 3-41). Furthermore, cancer cells have a natural proclivity to actively take up lipoproteins to acquire lipids needed for their rapid membrane turnover (Favre, Am J Obstet Gynecol, 139 (1981) 877-885; Gal et al., Am J Obstet Gynecol, 139 (1981) 877-885; Ho et al., Blood, 52 (1978) 1099-1114). The lipoprotein platform is also a particularly fitting vehicle for 7-DHC as it is analog of cholesterol, as these carriers naturally function to transport cholesterol in the plasma (Gotto et al., Methods Enzymol, 128 (1986) 3-41).

The low-density lipoprotein (LDL) nanocarrier described herein provides a favorable opportunity to deliver 7-DHC or a radiosensitizing analog or derivative thereof to cells of interest. The experiments in the Examples below, show that engineered 7-DHC@LDL nanoparticle retains several similar biochemical properties to control cholesterol@LDL particles, with the added benefit of functioning as a radiosensitizer. Thus, it is believed to be a fully biocompatible, biodegradable and non-immunogenic nanostructure. The LDL nanocarrier allows 7-DHC or derivative or analog thereof to be directly administered into the vascular system where it is protected by the LDL shell from any metabolic or degradative processes prior to arriving at its destination. ApoB-100 can be used on the surface of the LDL nanoparticle allows it to recognize the LDL receptor (LDLR) on cells. This is particularly important as many cancer cells overexpress the LDLR. Thus, through LDLR mediated endocytosis or 7-DHC or derivative or analog thereof @LDL can be used to increase accumulation of 7-DHC or derivative or analog thereof in cancer cells.

Lipoproteins are naturally occurring core-shell nanostructures that serve as the main transport vehicles for cholesterol and triglycerides in mammalian systems. The low-density lipoprotein (LDL) species, in particular, has drawn the attention of many cancer researchers due to the fact that many tumors over-express the LDL receptor (LDLR). Cancer cells are believed to express elevated levels of LDLR in order to compete with the host for the necessary cholesterol and fatty acids needed for active membrane turnover. In nature, LDL exhibit a fine particle size distribution (19-25 nm), have a high-payload carrying capacity (>1600 cholesterol esters and triglyceride molecules), circulate below MPS surveillance, and have a long serum half-life (2-4 days).

To date, progress with this approach has been impeded primarily by the inability to produce highly concentrated stable drug-LDL complexes. Many of the anticancer drugs used in these studies have been bulky heterocyclic compounds that have limited incorporation into the LDL platform (10-200 molecules per LDL) and readily ‘leak’ from the LDL in plasma. For these reasons the majority of the published reports have been limited to cell culture experiments and a few pre-clinical rodent studies. Unlike the previous listed anticancer agents, cholesterol is a natural cargo for LDL.

Compositions and methods for making reconstituted or synthetic lipoprotein and LDL-like particles are known in the art and can be adapted for incorporation of 7-DHC or a derivative or analog thereof. In some embodiments, the particles are prepared using art recognized compositions and other methods, however, some or all of the cholesterol is substituted for 7-DHC or a non-cholesterol derivative or analog thereof. In some embodiments, 7-DHC or a derivative or analog thereof is added in addition to cholesterol.

In some embodiments, the particles include a triglyceride and a phospholipid.

For example, U.S. Patent Application No. 2010/0297242, describes lipoprotein and lipoprotein-like particles composed of a lipid core part containing cholesteryl ester and triglyceride; and a cationic surface lipid part containing cholesterol, phospholipids and cationic lipids, which forms a cationic surface of the lipid core part via hydrophobic interaction.

In some embodiments, the LDL-like particle is free from protein.

Cholesteryl ester refers to cholesterol combined with saturated or unsaturated fatty acid having 10 to 24 carbon atoms by esterification. In some embodiments, the cholesteryl ester is ester of unsaturated fatty acid having 16 to 18 carbon atoms such as oleic acid. The nanoparticle can include single or plural kinds of cholesteryl esters.

Triglyceride include purified triglyceride having different compositions of various fatty acids or vegetable oils primarily containing triglyceride having plural fatty acids. Preferably, the triglyceride includes animal or vegetable oils and the vegetable oils may include soy bean oil, olive oil, cotton seed oil, sesame oil, liver oil and the like. Such oil may be used alone or in combination with two or more thereof. Examples include, but are not limited to, oleic acid (OA), and triolein (TO).

The cholesteryl ester and the triglyceride may form a lipid core part of the LDL-like cationic nanoparticle of the present invention through hydrophobic interaction.

Phospholipids can include any kind of neutral, cationic, and anionic phospholipids and, in addition, single or plural kinds of phospholipids. The phospholipids may include phosphatidyl choline (PC), phosphatidyl ethanolamine, phosphatidyl serine, phosphatidyl glycerol, lyso types of the above phospholipids, or fully saturated or partially hardened forms having aliphatic chains with 6 to 24 carbon atoms. The phospholipids are not particularly limited, however, may include at least one selected from a group consisting of: dioleoylphosphatidyl ethanolamine (DOPE); palmitoyloleoyl phosphatidyl choline (POPC); egg phosphatidyl choline (EPC); distearoylphosphatidyl choline (DSPC); dioleoylphosphatidyl choline (DOPC); dip almitoylphosphatidyl choline (DPPC); dioleoylphosphatidyl glycerol (DOPG); and dip almitoylphosphatidyl glycerol (DPPG). Other phospholipids are discussed below with respect to liposomes, and can also be used in lipoproteins particles. The cationic lipids include cationic lipids having a substantially positive charge at a specific pH such as physiological pH.

According to an exemplary embodiment, the cationic lipids may include at least one selected from a group consisting of: 3-beta-[N—(N′,N′,N′-trimethylaminoethane)carbamoyl]cholesterol (TC-cholesterol); 3-beta-[N—(N′,N′-dimethylaminoethane)carbamoyl]cholesterol (DC-cholesterol); 3-beta-[N—(N′-monomethylaminoethane)carbamoyl]cholesterol (MC-cholesterol), 3-beta-[N-(aminoethane)carbamoyl]cholesterol (AC-cholesterol); N—(N′-aminoethane)carbamoyl propanoic tocopherol (AC-tocopherol); N—(N′-methylaminoethane)carbamoyl propanoic tocopherol (MC-tocopherol); N,N-dioleyl-N,N-dimethylammonium chloride (DODAC); N,N-distearyl-N,N-dimethylammonium bromide (DDAB); N-(1-(2,3-dioleoyloxy)propyl-N,N,N-trimethylammonium chloride (DOTAP); N,N-dimethyl-(2,3-dioleoyloxy)propylamine (DODMA); N-(1-(2,3-dioleyloxy)propyl)-N,N,N-trimethylammonium chloride (DOTMA); 1,2-dioleoyl-3-dimethylammonium-propane (DODAP); 1,2-dioleoylcarbamyl-3-dimethylammonium-propane (DOCDAP); 1,2-dilineoyl-3-dimethylammonium-propane (DLINDAP); dioleoyloxy-N-[2-(sperminecarboxamido)ethyl]-N,N-dimethyl-1-propanaminium-trifluoroacetate (DOSPA); dioctadecylamidoglycyl spermine (DOGS); 1,2-dimyristyloxypropyl-3-dimethyl-hydroxyethyl ammonium bromide (DMRIE), 3-dimethylamino-2-(cholest-5-en-3-beta-oxybutan-4-oxy)-1-(cis,cis-9,12-oc-tadecadien oxy)propane (CLinDMA); 2-[5′-(cholest-5-en-3-beta-oxy)-3′-oxapentoxy]-3-dimethyl-1-(cis,cis-9′,1-2′-octadecadienoxy)propane (CpLinDMA); N,N-dimethyl-3,4-dioleyloxybenzylamine (DMOBA); 1,2-N,N′-dioleylcarbamyl-3-dimethylaminopropane (DOcarbDAP); 1,2-diacyl-3-trimethylammonium-propane (TAP); and 1,2-diacyl-3-dimethylammonium-propane (DAP). Thus, the nanoparticle can be an LDL-like cationic nanoparticle.

A natural LDL typically has two lipid phases, that is, polar constituents (phospholipids and apolipoproteins) and non-polar neutral lipids previously consisting of cholesterol ester and triglyceride, and composition and physicochemical characteristics thereof are shown in Table 1. Phospholipid and apolipoprotein emulsify non-polar lipid to ensure stability of a surface thereof, thereby forming a stable bio-microemulsion.

Table 1 Compositions of Natural LDL and physicochemical characteristics thereof Natural LDL Section Ingredients Content (w/w) lipid Core part Cholesteryl ester 45% Triglyceride 3% Surface part Cholesterol 10% Phospholipid 22% Apolipoprotein B-100 20% Size (nm) 18 to 25 Zeta potential (mV) −11.4.+−. 1.9

TABLE 1
Compositions of Natural LDL and physiochemical
characteristics thereof

| | Natural LDL | |
|---|---|---|
| | | Content |
| Section | Ingredients | (w/w) |
| lipid Core part | Cholesteryl ester | 45% |
| | Triglyceride | 3% |
| Surface part | Cholesterol | 10% |
| | Phospholipid | 22% |
| | Apolipoprotein B-100 | 20% |

| | |
|---|---|
| Size (nm) | 18 to 25 |
| Zeta potential (mV) | −11.4 ± 1/9 [25] |

In some embodiments, an LDL-like nanoparticle for delivering 7-DHC or a derivative or analog thereof includes: 30 to 60 wt. % of cholesteryl ester; 0.1 to 10 wt. % of triglyceride; 5 to 20 wt. % of cholesterol; 5 to 30 wt. % of phospholipids; and 10 to 50 wt. % of lipid, e.g., cationic lipid.

In some embodiments, a weight ratio of the lipid core part to the surface lipid part in the nanoparticle ranges from 30:70 to 70:30 relative to weight of a nanoparticle carrier.

See also U.S. Published Application No. 2010/0047163, which is specifically incorporated by reference herein in its entirety.

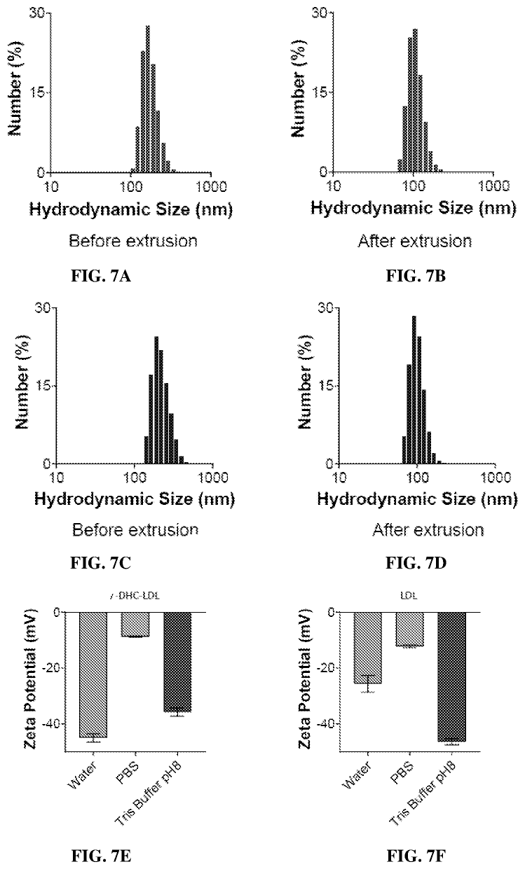

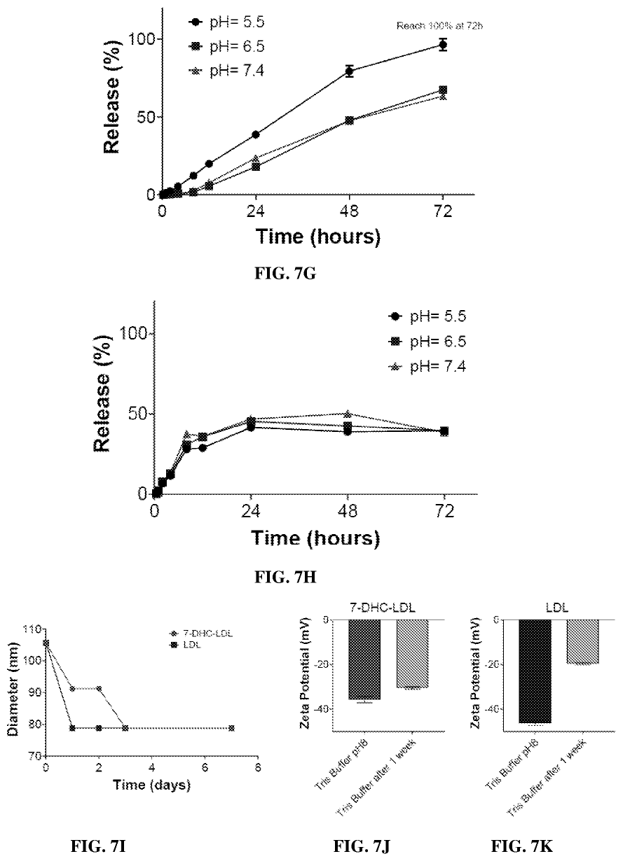

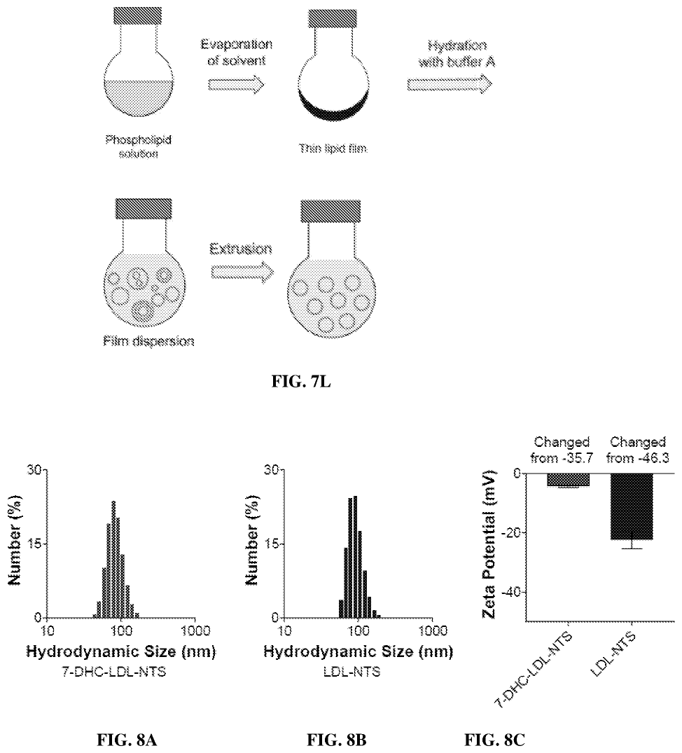

In the experiments below, phosphatidyl choline (PC), triolein (TO), and 7 DHC (e.g., in a 3:2:1 molar ratio) were introduced to solvent (e.g., 2:1 v/v CHCl₃:MeOH) and mixed in a round bottom flask, rotary evaporation at 60° C. Buffer, (e.g., Tris buffer) and stirring was continued at 55-65° C. Particles were extruded though a 100 nm membrane at 55-65° C. Particles were stored at 4° C. See also FIG. 7L. In other embodiments, TO is used in combination with, or substituted with, oleic acid (OA).

In other embodiments, isolated LDL lipoprotein, e.g., from a human source, is utilized. See, e.g., U.S. Published Application No. 2016/0015636, which is specifically incorporated by reference herein in its entirety. For example, LDL can be isolated from apheresis plasma of patients using sequential density gradient ultracentrifugation as described previously by Lund-Katz et al. (Lund-Katz et al., Biochemistry, 37 (1998) 12867-12874). Incorporation of 7-DHC or a derivative or analog thereof into LDL can be performed by the reconstitution (core loading) method (Krieger, Methods Enzymol., 128 (1986) 608-613). Briefly, lyophilized LDL is subjected to organic extraction with heptane. Following the extraction, 7-DHC or a derivative or analog thereof is added to the LDL residue and the sample is allowed to sit at 4° C. Thereafter, heptane can be removed by evaporation and the dried residue resuspended in buffer, e.g., Tricine buffer. LDL samples can be clarified by low-speed centrifugation. Control LDL Nanoparticles: Throughout these studies, various LDL particles were used as controls. These included native LDL as an overall control vehicle; LDL reconstituted with oleic acid (LDL-OA), or LDL reconstituted with oleic acid triglyceride (triolein) (LDL-TO).

The particles can, but need not necessarily, include protein. In some embodiments, the particle includes one or plural kinds of apoproteins. The apoprotein can be extracted from a natural lipoprotein or produced by recombination of proteins. Preferred examples of the apoprotein may include B-100, apo E, etc. Such an apoprotein may allow the nanoparticle of the to be efficiently introduced into cells in a specific mode.

c. Liposomes and Micelles

In some forms, the compounds can be encapsulated in liposomal vesicles, lipid micelles, or solid lipid nanoparticles, or a combination thereof. The nanoparticles can contain one or more lipids or amphiphilic compounds. The nanoparticles are preferably made from one or more biocompatible lipids. The nanoparticles can be made from one or a mixture of different lipids that can be neutral, anionic, or cationic at physiologic pH (such as pH 7.4). As a non-limiting example, a charged lipid may be combined with a lipid that is non-ionic or uncharged at physiological pH.

In some forms, the nanoparticle can be a lipid micelle. Lipid micelles for drug delivery are known in the art. Lipid micelles can be formed, for instance, as a water-in-oil emulsion with a lipid surfactant. An emulsion is a blend of two immiscible phases wherein a surfactant is added to stabilize the dispersed droplets. The lipid micelle can be a microemulsion. A microemulsion is a thermodynamically stable system composed of at least water, oil, and a lipid surfactant producing a transparent and thermodynamically stable system whose droplet size is less than 1 micron, from about 10 nm to about 500 nm, or from about 10 nm to about 250 nm. Lipid micelles are generally useful for encapsulating hydrophobic active agents, including hydrophobic therapeutic agents, hydrophobic prophylactic agents, or hydrophobic diagnostic agents.

In some forms, the nanoparticle can be a liposome, such as a liposomal vesicle. Liposomal vesicles typically contain an aqueous medium surrounded by lipids arranged in spherical bilayers. Liposomal vesicles can be classified as small unilamellar vesicles, large unilamellar vesicles, or multi-lamellar vesicles. Multi-lamellar liposomes contain multiple concentric lipid bilayers. Liposomes can be used to encapsulate therapeutic, diagnostic, and or prophylactic agents, by trapping hydrophilic agents in the aqueous interior or between bilayers, or by trapping hydrophobic agents within the bilayer.

The lipid micelles and liposomes typically have an aqueous center. The aqueous center can contain water or a mixture of water and alcohol. Suitable alcohols include, but are not limited to, methanol, ethanol, propanol, (such as isopropanol), butanol (such as n-butanol, isobutanol, sec-butanol, tert-butanol, pentanol (such as amyl alcohol, isobutyl carbinol), hexanol (such as 1-hexanol, 2-hexanol, 3-hexanol), heptanol (such as 1-heptanol, 2-heptanol, 3-heptanol and 4-heptanol) or octanol (such as 1-octanol) or a combination thereof.

In some forms, the nanoparticle can be a solid lipid nanoparticle. Solid lipid nanoparticles present an alternative to the colloidal micelles and liposomal vesicles. Solid lipid nanoparticles are typically submicron in size, i.e. from about 10 nm to about 1 micron, from 10 nm to about 500 nm, or from 10 nm to about 250 nm. Solid lipid nanoparticles can be formed of lipids that are solids at room temperature. They are derived from oil-in-water emulsions, by replacing the liquid oil by a solid lipid.

Suitable neutral and anionic lipids include, but are not limited to, sterols and lipids such as cholesterol, phospholipids, lysolipids, lysophospholipids, sphingolipids or pegylated lipids. Neutral and anionic lipids include, but are not limited to, phosphatidylcholine (PC) (such as egg PC, soy PC), including 1,2-diacyl-glycero-3-phosphocholines; phosphatidylserine (PS), phosphatidylglycerol, phosphatidylinositol (PI); glycolipids; sphingophospholipids such as sphingomyelin and sphingoglycolipids (also known as 1-ceramidyl glucosides) such as ceramide galactopyranoside, gangliosides and cerebrosides; fatty acids, sterols, containing a carboxylic acid group for example, cholesterol; 1,2-diacyl-sn-glycero-3-phosphoethanolamine, including, but not limited to, 1,2-dioleylphosphoethanolamine (DOPE), 1,2-dihexadecylphosphoethanolamine (DHPE), 1,2-distearoylphosphatidylcholine (DSPC), 1,2-dipalmitoyl phosphatidylcholine (DPPC), and 1,2-dimyristoylphosphatidylcholine (DMPC). The lipids can also include various natural (e.g., tissue derived L-.alpha.-phosphatidyl: egg yolk, heart, brain, liver, soybean) and/or synthetic (e.g., saturated and unsaturated 1,2-diacyl-sn-glycero-3-phosphocholines, 1-acyl-2-acyl-sn-glycero-3-phosphocholines, 1,2-diheptanoyl-SN-glycero-3-phosphocholine) derivatives of the lipids.

Suitable cationic lipids include, but are not limited to, N-[1-(2,3-dioleoyloxy)propyl]-N,N,N-trimethyl ammonium salts, also references as TAP lipids, for example methylsulfate salt. Suitable TAP lipids include, but are not limited to, DOTAP (dioleoyl-), DMTAP (dimyristoyl-), DPTAP (dipalmitoyl-), and DSTAP (distearoyl-). Suitable cationic lipids in the liposomes include, but are not limited to, dimethyldioctadecyl ammonium bromide (DDAB), 1,2-diacyloxy-3-trimethylammonium propanes, N-[1-(2,3-dioloyloxy)propyl]-N,N-dimethyl amine (DODAP), 1,2-diacyloxy-3-dimethylammonium propanes, N-[1-(2,3-dioleyloxy)propyl]-N,N,N-trimethylammonium chloride (DOTMA), 1,2-dialkyloxy-3-dimethylammonium propanes, dioctadecylamidoglycylspermine (DOGS), 3-[N—(N′,N-dimethylamino-ethane)carbamoyl]cholesterol (DC-Chol); 2,3-dioleoyloxy-N-(2-(sperminecarboxamido)-ethyl)-N,N-dimethyl-1-propanaminium trifluoro-acetate (DOSPA), beta-alanyl cholesterol, cetyl trimethyl ammonium bromide (CTAB), diC₁₄-amidine, N-ferf-butyl-N′-tetradecyl-3-tetradecylamino-propionamidine, N-(alpha-trimethylammonioacetyl)didodecyl-D-glutamate chloride (TMAG), ditetradecanoyl-N-(trimethylammonio-acetyl)diethanolamine chloride, 1,3-dioleoyloxy-2-(6-carboxy-spermyl)-propylamide (DOSPER), and N,N,N′,N′-tetramethyl-, N′-bis(2-hydroxylethyl)-2,3-dioleoyloxy-1,4-butanediammonium iodide. In one embodiment, the cationic lipids can be 1-[2-(acyloxy)ethyl]2-alkyl(alkenyl)-3-(2-hydroxyethyl)-imidazolinium chloride derivatives, for example, 1-[2-(9(Z)-octadecenoyloxy)ethyl]-2-(8(Z)-heptadecenyl-3-(2-hydroxyethyl)-imidazolinium chloride (DOTIM), and 1-[2-(hexadecanoyloxy)ethyl]-2-pentadecyl-3-(2-hydroxyethyl)imidazolinium chloride (DPTIM). In one embodiment, the cationic lipids can be 2,3-dialkyloxypropyl quaternary ammonium compound derivatives containing a hydroxyalkyl moiety on the quaternary amine, for example, 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimethyl-hydroxypropyl ammonium bromide (DORIE-HP), 1,2-dioleyl-oxy-propyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), and 1,2-disteryloxypropyl-3-dimethyl-hydroxyethyl ammonium bromide (DSRIE).

Suitable solid lipids include, but are not limited to, higher saturated alcohols, higher fatty acids, sphingolipids, synthetic esters, and mono-, di-, and triglycerides of higher saturated fatty acids. Solid lipids can include aliphatic alcohols having 10-40, preferably 12-30 carbon atoms, such as cetostearyl alcohol. Solid lipids can include higher fatty acids of 10-40, preferably 12-30 carbon atoms, such as stearic acid, palmitic acid, decanoic acid, and behenic acid. Solid lipids can include glycerides, including monoglycerides, diglycerides, and triglycerides, of higher saturated fatty acids having 10-40, preferably 12-30 carbon atoms, such as glyceryl monostearate, glycerol behenate, glycerol palmitostearate, glycerol trilaurate, tricaprin, trilaurin, trimyristin, tripalmitin, tristearin, and hydrogenated castor oil. Suitable solid lipids can include cetyl palmitate, beeswax, or cyclodextrin.

Amphiphilic compounds include, but are not limited to, phospholipids, such as 1,2 distearoyl-sn-glycero-3-phosphoethanolamine (DSPE), dipalmitoylphosphatidylcholine (DPPC), distearoylphosphatidylcholine (DSPC), diarachidoylphosphatidylcholine (DAPC), dibehenoylphosphatidylcholine (DBPC), ditricosanoylphosphatidylcholine (DTPC), and dilignoceroylphatidylcholine (DLPC), incorporated at a ratio of between 0.01-60 (weight lipid/w polymer), most preferably between 0.1-30 (weight lipid/w polymer). Phospholipids which may be used include, but are not limited to, phosphatidic acids, phosphatidyl cholines with both saturated and unsaturated lipids, phosphatidyl ethanolamines, phosphatidylglycerols, phosphatidylserines, phosphatidylinositols, lysophosphatidyl derivatives, cardiolipin, and .beta.-acyl-y-alkyl phospholipids. Examples of phospholipids include, but are not limited to, phosphatidylcholines such as dioleoylphosphatidylcholine, dimyristoylphosphatidylcholine, dipentadecanoylphosphatidylcholine dilauroylphosphatidylcholine, dipalmitoylphosphatidylcholine (DPPC), distearoylphosphatidylcholine (DSPC), diarachidoylphosphatidylcholine (DAPC), dibehenoylphosphatidylcho-line (DBPC), ditricosanoylphosphatidylcholine (DTPC), dilignoceroylphatidylcholine (DLPC); and phosphatidylethanolamines such as dioleoylphosphatidylethanolamine or 1-hexadecyl-2-palmitoylglycerophos-phoethanolamine. Synthetic phospholipids with asymmetric acyl chains (e.g., with one acyl chain of 6 carbons and another acyl chain of 12 carbons) may also be used.

In some embodiments, the liposomes can be coated with a water-soluble, biocompatible polymer. Suitable polymers include, but are not limited to polyalkylene oxides such as polyethylene glycol (PEG), polyethylene glycol-polypropylene block copolymer such as a PLURONIC®, poly(N-isopropylacrylamide) (PNIPAM), polyacrylamide (PAM), poly(carboxybetaine)s (pCB), poly(sulfobetaine)s (pSB), poly(phosphobetaine)s, and polyethyleneimine (PEI). In some forms, the polymer can be polyethylene glycol forming coated liposomes collectively known as PEGylated liposomes.

d. Inorganic Nanoparticles

In some forms, the particles can be of an inorganic composition including, but not limited to, minerals, including silica, silicates; sulfides (such as bismuth sulfide (Bi₂S₃), gold bismuth sulfide (Au-siBi₂S₃), oxides, halides, carbonates, sulfates, phosphates; iron(II) oxide, iron(III) oxide. In some forms, the particles can also be made of one or more metals, such as gold nanoparticles, silver nanoparticles, copper, platinum, palladium, ruthenium, or a combination thereof.

### 2. Targeting Agents

The 7-DHC or derivative or analog thereof and/or nanoparticles for delivery of the compounds can also include a targeting agent. A targeting agent can be a peptide, nucleic acid, glycoprotein, carbohydrate, lipid, or small molecule that binds to one or more targets associated with an organ, tissue, cell, subcellular locale, or extracellular matrix.

In some forms, one or more targeting agents can be conjugated to the compounds or nanoparticles, preferably covalently. The targeting agents can be covalently associated with the compounds or nanoparticles, directly or indirectly via a linker. Although discussed herein primarily as attaching a targeting agent to nanoparticles, in some embodiments 7-DHC or a derivative or analog thereof is coupled to a targeting agent, e.g., a peptide or protein, with an appropriate linker and used without nanoparticles. Preferably the targeting agent does not interfere with compound's activity and the compound does not interfere with receptor binding. It is believed that if the spacer is long enough, the drug's interference with receptor binding would be low.

In some embodiments, coupling of the target agent to the compounds or nanoparticles is achieved by linking a sulfhydryl (—SH) (e.g., on a cysteine) and an amine using, e.g., Sulfosuccinimidyl-4-(N-maleimidomethyl) cyclohexane-1-carboxylate (Sulfo-SMCC) crosslinker. For example, the compounds or particles can have a free amine (e.g., PLGA-b-PEG-amine) and the targeting agent can have a cysteine with an —SH available for crosslinking. In other embodiments, a carboxyl-to-amine crosslinker such as EDC/NHS can be used. In a particular example, the particles have a free carboxyl (e.g., PLGA-b-PEG-COOH) and the targeting agent has a free amine. Click chemistry can also be used for the coupling. A common example is azide-alkyne coupling through copper(I) catalysis or copper free strain-promoted cycloaddition. Strain-promoted alkyne-nitrone cycloaddition is also feasible. Adize and alkyne can be separately coupled to compounds or nanoparticles and targeting ligands, and click chemistry will link the two components together.

The particles can be composed of a mixture of polymers, e.g., with and without the moiety used to couple the targeting agent. By non-limiting example, the particles may be formed of a mixture including PLGA-b-PEG and PLGA-b-PEG-amine, or a mixture of PLGA-b-PEG and PLGA-b-PEG-COOH. Any suitable ratio of polymers can be used, and can be used to tune the relative number of targeting agents presented on the surface of the nanoparticle (i.e., ligand surface density). For example, the molar ratio of polymer with the coupling moiety to polymer without coupling moiety can be X:Y wherein X and Y are independently any integer from 1-100 inclusive. In an exemplary embodiment, non-limiting embodiment, the molar ratio of polymer with the coupling moiety to polymer without coupling moiety is 1:10. The peptide-to-particle ratio can then be calculated (Derman, et al., J Biomed Sci, 22, 89 (2015)).

Preferably, the targeting agent binds to a molecule (targeted moiety) that is specific to tumor cells, or may be expressed at a higher level on tumor cells as compared to non-tumor cells.

Examples of targeting agents include peptides such as iRGD, NGR, iNGR, RGR, LyP1; small molecules such as folate; aptamers, antibodies, antigen binding fragment or fusion proteins of an antibody.

Exemplary antibodies and fragments that can be used include monoclonal and polyclonal antibodies, single chain antibodies, single chain variable fragments (scFv), di-scFv, tri-scFv, diabody, triabody, teratbody, disulfide-linked Fvs (sdFv), Fab′, F(ab′)₂, Fv, and single domain antibody fragments (sdAb). The antibodies can bind to targets on cancer cells or in a tumor's microenvironment. Exemplary cancer antigen targets are discussed below.

Examples of targeting peptides are described in U.S. Pat. Nos. 6,177,542, 7,544,767, and 6,576,239; U.S. Patent Application Publication No. 20090257951; and Hoffman, et al., Cancer Cell, vol. 4 (2003). Useful NGR peptides include peptide such as X₂CNGRCX₂ (SEQ ID NO: 1), CX₂(C/X)NGR(C/X)X₂C (SEQ ID NO:2), and CNGRCX₆ (SEQ ID NO:3) (where “X” is any amino acid), which can be linear or circular.

Useful peptides for tumor targeting include, for example, iRGD, LyP-1, iNGR, and RGR peptides. iRGD has a unique target within tumors; it preferentially accumulates in the hypoxic/low nutrient areas of tumors (Laakkonen, et al., 2002; 2004; Karmali, et al., 2009). CRGRRST (SEQ ID NO:4) (RGR; Joyce, et al., 2003) is a peptide that has been successfully used in targeting a cytokine antibody combination into tumors (Hamzah, et al., 2008). This peptide is linear, which simplifies the synthesis. NGR peptides home to angiogenic vasculature, including angiogenic vasculature associated with tumors, and αᵥ integrin and α₅β₁ integrin (U.S. Pat. Nos. 6,576,239 and 6,177,542 and U.S. Patent Application Publication No. 20090257951).

In some forms, the targeted moiety is an antigen that is expressed by tumor cells. Antigenic markers such as serologically defined markers known as tumor associated antigens, which are either uniquely expressed by cancer cells or are present at markedly higher levels (e.g., elevated in a statistically significant manner) in subjects having a malignant condition relative to appropriate controls, are known.

Tumor-associated antigens may include, for example, cellular oncogene-encoded products or aberrantly expressed proto-oncogene-encoded products (e.g., products encoded by the neu, ras, trk, and kit genes), or mutated forms of growth factor receptor or receptor-like cell surface molecules (e.g., surface receptor encoded by the c-erb B gene). Other tumor-associated antigens include molecules that may be directly involved in transformation events, or molecules that may not be directly involved in oncogenic transformation events but are expressed by tumor cells (e.g., carcinoembryonic antigen, CA-125, melanoma associated antigens, etc.) (see, e.g., U.S. Pat. No. 6,699,475; Jager, et al., Int. J. Cancer, 106:817-20 (2003); Kennedy, et al., Int. Rev. Immunol., 22:141-72 (2003); Scanlan, et al., Cancer Immun., 4:1 (2004)).

Genes that encode cellular tumor associated antigens include cellular oncogenes and proto-oncogenes that are aberrantly expressed. In general, cellular oncogenes encode products that are directly relevant to the transformation of the cell, so these antigens are particularly preferred targets for immunotherapy. An example is the tumorigenic neu gene that encodes a cell surface molecule involved in oncogenic transformation. Other examples include the ras, kit, and trk genes. The products of proto-oncogenes (the normal genes which are mutated to form oncogenes) may be aberrantly expressed (e.g., overexpressed), and this aberrant expression can be related to cellular transformation. Thus, the product encoded by proto-oncogenes can be targeted. Some oncogenes encode growth factor receptor molecules or growth factor receptor-like molecules that are expressed on the tumor cell surface. An example is the cell surface receptor encoded by the c-erbB gene. Other tumor-associated antigens may or may not be directly involved in malignant transformation. These antigens, however, are expressed by certain tumor cells and may therefore provide effective targets. Some examples are carcinoembryonic antigen (CEA), CA 125 (associated with ovarian carcinoma), and melanoma specific antigens.

In ovarian and other carcinomas, for example, tumor associated antigens are detectable in samples of readily obtained biological fluids such as serum or mucosal secretions. One such marker is CA125, a carcinoma associated antigen that is also shed into the bloodstream, where it is detectable in serum (e.g., Bast, et al., N. Eng. J. Med., 309:883 (1983); Lloyd, et al., Int. J. Canc., 71:842 (1997). CA125 levels in serum and other biological fluids have been measured along with levels of other markers, for example, carcinoembryonic antigen (CEA), squamous cell carcinoma antigen (SCC), tissue polypeptide specific antigen (TPS), sialyl TN mucin (STN), and placental alkaline phosphatase (PLAP), in efforts to provide diagnostic and/or prognostic profiles of ovarian and other carcinomas (e.g., Sarandakou, et al., Acta Oncol., 36:755 (1997); Sarandakou, et al., Eur. J. Gynaecol. Oncol., 19:73 (1998); Meier, et al., Anticancer Res., 17(4B):2945 (1997); Kudoh, et al., Gynecol. Obstet. Invest., 47:52 (1999)). Elevated serum CA125 may also accompany neuroblastoma (e.g., Hirokawa, et al., Surg. Today, 28:349 (1998), while elevated CEA and SCC, among others, may accompany colorectal cancer (Gebauer, et al., Anticancer Res., 17(4B):2939 (1997)).

The tumor associated antigen mesothelin, defined by reactivity with monoclonal antibody K-1, is present on a majority of squamous cell carcinomas including epithelial ovarian, cervical, and esophageal tumors, and on mesotheliomas (Chang, et al., Cancer Res., 52:181 (1992); Chang, et al., Int. J. Cancer, 50:373 (1992); Chang, et al., Int. J. Cancer, 51:548 (1992); Chang, et al., Proc. Natl. Acad. Sci. USA, 93:136 (1996); Chowdhury, et al., Proc. Natl. Acad. Sci. USA, 95:669 (1998)). Using MAb K-1, mesothelin is detectable only as a cell-associated tumor marker and has not been found in soluble form in serum from ovarian cancer patients, or in medium conditioned by OVCAR-3 cells (Chang, et al., Int. J. Cancer, 50:373 (1992)). Structurally related human mesothelin polypeptides, however, also include tumor-associated antigen polypeptides such as the distinct mesothelin related antigen (MRA) polypeptide, which is detectable as a naturally occurring soluble antigen in biological fluids from patients having malignancies (see WO 00/50900).

A tumor antigen may include a cell surface molecule. Tumor antigens of known structure and having a known or described function, include the following cell surface receptors: HER1 (GenBank Accession NO: U48722), HER2 (Yoshino, et al., J. Immunol., 152:2393 (1994); Disis, et al., Canc. Res., 54:16 (1994); GenBank Acc. Nos. X03363 and M17730), HER3 (GenBank Acc. Nos. U29339 and M34309), HER4 (Plowman, et al., Nature, 366:473 (1993); GenBank Acc. Nos. L07868 and T64105), epidermal growth factor receptor (EGFR) (GenBank Acc. Nos. U48722, and KO3193), vascular endothelial cell growth factor (GenBank NO: M32977), vascular endothelial cell growth factor receptor (GenBank Acc. Nos. AF022375, 1680143, U48801 and X62568), insulin-like growth factor-I (GenBank Acc. Nos. X00173, X56774, X56773, X06043, European Patent No. GB 2241703), insulin-like growth factor-II (GenBank Acc. Nos. X03562, X00910, M17863 and M17862), transferrin receptor (Trowbridge and Omary, Proc. Nat. Acad. USA, 78:3039 (1981); GenBank Acc. Nos. X01060 and M11507), estrogen receptor (GenBank Acc. Nos. M38651, X03635, X99101, U47678 and M12674), progesterone receptor (GenBank Acc. Nos. X51730, X69068 and M15716), follicle stimulating hormone receptor (FSH—R) (GenBank Acc. Nos. Z34260 and M65085), retinoic acid receptor (GenBank Acc. Nos. L12060, M60909, X77664, X57280, X07282 and X06538), MUC-1 (Barnes, et al., Proc. Nat. Acad. Sci. USA, 86:7159 (1989); GenBank Acc. Nos. M65132 and M64928) NY-ESO-1 (GenBank Acc. Nos. AJ003149 and U87459), NA 17-A (PCT Publication NO: WO 96/40039), Melan-A/MART-1 (Kawakami, et al., Proc. Nat. Acad. Sci. USA, 91:3515 (1994); GenBank Acc. Nos. U06654 and U06452), tyrosinase (Topalian, et al., Proc. Nat. Acad. Sci. USA, 91:9461 (1994); GenBank Acc. NO: M26729; Weber, et al., J. Clin. Invest, 102:1258 (1998)), Gp-100 (Kawakami, et al., Proc. Nat. Acad. Sci. USA, 91:3515 (1994); GenBank Acc. NO: S73003, Adema, et al., J. Biol. Chem., 269:20126 (1994)), MAGE (van den Bruggen, et al., Science, 254:1643 (1991)); GenBank Acc. Nos. U93163, AF064589, U66083, D32077, D32076, D32075, U10694, U10693, U10691, U10690, U10689, U10688, U10687, U10686, U10685, L18877, U10340, U10339, L18920, U03735 and M77481), BAGE (GenBank Acc. NO: U19180; U.S. Pat. Nos. 5,683,886 and 5,571,711), GAGE (GenBank Acc. Nos. AF055475, AF055474, AF055473, U19147, U19146, U19145, U19144, U19143 and U19142), any of the CTA class of receptors including in particular HOM-MEL-40 antigen encoded by the SSX2 gene (GenBank Acc. Nos. X86175, U90842, U90841 and X86174), carcinoembryonic antigen (CEA, Gold and Freedman, J. Exp. Med., 121:439 (1985); GenBank Acc. Nos. M59710, M59255 and M29540), and PyLT (GenBank Acc. Nos. J02289 and J02038); p97 (melanotransferrin) (Brown, et al., J. Immunol., 127:539-46 (1981); Rose, et al., Proc. Natl. Acad. Sci. USA, 83:1261-61 (1986)).

Additional tumor associated antigens include prostate surface antigen (PSA) (U.S. Pat. Nos. 6,677,157; 6,673,545); β-human chorionic gonadotropin β-HCG) (McManus, et al., Cancer Res., 36:3476-81 (1976); Yoshimura, et al., Cancer, 73:2745-52 (1994); Yamaguchi, et al., Br. J. Cancer, 60:382-84 (1989): Alfthan, et al., Cancer Res., 52:4628-33 (1992)); glycosyltransferase β-1,4-N-acetylgalactosaminyltransferases (GalNAc) (Hoon, et al., Int. J. Cancer, 43:857-62 (1989); Ando, et al., Int. J. Cancer, 40:12-17 (1987); Tsuchida, et al., J. Natl. Cancer, 78:45-54 (1987); Tsuchida, et al., J. Natl. Cancer, 78:55-60 (1987)); NUC18 (Lehmann, et al., Proc. Natl. Acad. Sci. USA, 86:9891-95 (1989); Lehmann, et al., Cancer Res., 47:841-45 (1987)); melanoma antigen gp75 (Vijayasardahi, et al., J. Exp. Med., 171:1375-80 (1990); GenBank Accession NO: X51455); human cytokeratin 8; high molecular weight melanoma antigen (Natali, et al., Cancer, 59:55-63 (1987); keratin 19 (Datta, et al., J. Clin. Oncol., 12:475-82 (1994)).

Tumor antigens of interest include antigens regarded in the art as “cancer/testis” (CT) antigens that are immunogenic in subjects having a malignant condition (Scanlan, et al., Cancer Immun., 4:1 (2004)). CT antigens include at least 19 different families of antigens that contain one or more members and that are capable of inducing an immune response, including, but not limited to, MAGEA (CT1); BAGE (CT2); MAGEB (CT3); GAGE (CT4); SSX (CT5); NY-ESO-1 (CT6); MAGEC (CT7); SYCP1 (C8); SPANXBI (CT11.2); NA88 (CT18); CTAGE (CT21); SPA17 (CT22); OY-TES-1 (CT23); CAGE (CT26); HOM-TES-85 (CT28); HCA661 (CT30); NY-SAR-35 (CT38); FATE (CT43); and TPTE (CT44).

Additional tumor antigens that can be targeted, including a tumor-associated or tumor-specific antigen, include, but are not limited to, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-1, 2, and 3, neo-PAP, myosin class I, OS-9, pml-RARα fusion protein, PTPRK, K-ras, N-ras, Triosephosphate isomeras, Bage-1, Gage 3,4,5,6,7, GnTV, Herv-K-mel, Lage-1, Mage-A1,2,3,4,6,10,12, Mage-C2, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, α-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\70K, NY—CO-1, RCAS1, SDCCAG16, TA-90 (Mac-2 binding protein\cyclophilin C-associated protein), TAAL6, TAG72, TLP, and TPS. Other tumor-associated and tumor-specific antigens are known to those of skill in the art and are suitable for targeting by the disclosed fusion proteins.

In some forms, antigens associated with tumor neovasculature can also be targeted. Tumor-associated neovasculature provides a readily accessible route through which therapeutics can access the tumor. In one embodiment the viral proteins contain a domain that specifically binds to an antigen that is expressed by neovasculature associated with a tumor.

The antigen may be specific to tumor neovasculature or may be expressed at a higher level in tumor neovasculature when compared to normal vasculature. Exemplary antigens that are over-expressed by tumor-associated neovasculature as compared to normal vasculature include, but are not limited to, VEGF/KDR, Tie2, vascular cell adhesion molecule (VCAM), endoglin and α₅β₃ integrin/vitronectin. Other antigens that are over-expressed by tumor-associated neovasculature as compared to normal vasculature are known to those of skill in the art and are suitable for targeting by the disclosed fusion proteins.

Neurotensin receptor 1 (NTSR1) is upregulated in the majority of lung tumors (59.7%), but expressed at low or undetectable levels in normal pulmonary tissues (Alifano, et al., Clinical Cancer Research, 16, 4401-4410 (2010)). NTSR1 upregulation is associated with poor 5-year overall survival, high metastasis rate, and increased neuroendocrine differentiation (Alifano, et al., Clinical Cancer Research, 16, 4401-4410 (2010), Dupouy, Biochimie, 93, 1369-78 (2011)). Thus, in some embodiments, the disclosed compositions, e.g, nanoparticles, include a ligand for NTSR1 coupled thereto.

NTSR1 is also upregulated in head and neck, breast, and colon cancer. Thus, in some embodiments, the NTSR1-targeting compositions are used for treatment of a cancer with upregulated NTSR1. In some embodiments, the subject has a lung cancer such as NSCLC, a head and neck cancer, a breast cancer, and/or a colon cancer.

In some embodiments, the NTSR1 is the wildtype NTSR1 ligand, neurotensin (NTS), or a variant, analog, or functional fragment thereof.

A wildtype sequence for human NTS is QLYENKPRRPYIL (SEQ ID NO:5), UniProtKB—P30990 (NEUT_HUMAN)—amino acids 151-163, which is specifically incorporated by reference herein in its entirety. In some embodiments, the NTSR1 ligand includes at least 50, 55, 60, 65, 70, 75, 80, 85, 90, or 95% sequence identity to SEQ ID NO:5. Neurotensin shares significant sequence similarity in its 6 C-terminal amino acids with several other neuropeptides, including neuromedin N (which is derived from the same precursor) (see, e.g., UniProtKB—P30990 (NEUT_HUMAN)). This C-terminal region is responsible for the full biological activity, the N-terminal portion having a modulatory role.

The neurotensin/neuromedin N precursor can also be processed to produce large 125-138 amino acid peptides with the neurotensin or neuromedin N sequence at their C terminus. These large peptides appear to be less potent than their smaller counterparts, but are also less sensitive to degradation and may represent endogenous, long-lasting activators in a number of pathophysiological situations.

NTS has a short half-life in vivo due to peptidase degradation (Reinecke, et al., Prog Histochem Cytochem, 16, 1-172 (1985), Wu, et al., J Nucl Med, 55, 1178-1184 (2014)). Thus, in some embodiments, the NTSR1 ligand is an NTS analog. An exemplary NTS analog is NTSₘᵤₜ. NTSₘᵤₜ includes amino acids 7-13 of SEQ ID NO:5, (e.g., PRRPYIL (SEQ ID NO:6) where unnatural amino acids are introduced to stabilize the bonds between Arg8-Arg9, Pro10-Tyr11, and Tyr11-Ile12.

A structure of an NTSₘᵤₜ with a terminal cysteine to facilitate coupling of the peptide to nanoparticles or compounds is provided below:

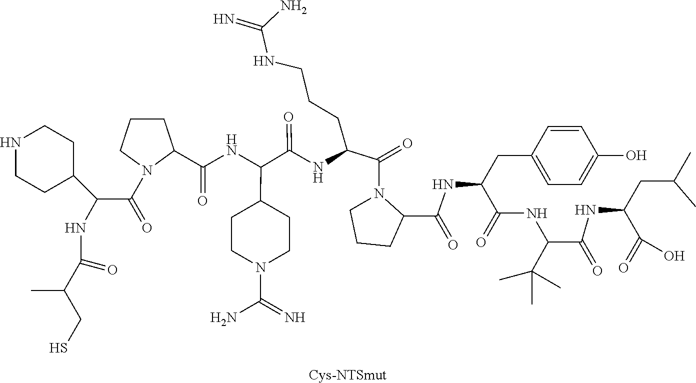 [SMILES: CC(C)C[C@H](NC(=O)C(NC(=O)[C@H](Cc1ccc(O)cc1)NC(=O)C1CCCN1C(=O)[C@H](CCCNC(=N)N)NC(=O)C(NC(=O)C1CCCN1C(=O)C(NC(=O)C(C)CS)C1CCNCC1)C1CCN(C(=N)N)CC1)C(C)(C)C)C(=O)O ; "Cys-NTSmut"]

See also, e.g., Wu, et al., J Nucl Med, 55(7):1178-84 (2014) doi: 10.2967/jnumed.114.137489, which is specifically incorporated by reference herein in its entirety.

Compared to wildtype NTS, NTSₘᵤₜ affords comparable, nanomolar avidity to NTSR1, but much greater biological stability. Increased surface ligand density may enhance binding affinity to NTSR1, but may also increase surface hydrophobicity that may negatively affects pharmacokinetics.

In another embodiment, the ligand is NTS_{20.8}, e.g., as illustrated below with a cysteine to facilitate coupling of the peptide to nanoparticles or or one or its derivatives.

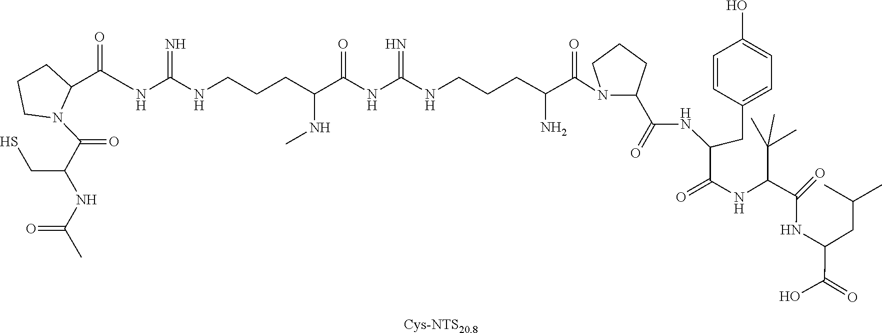 [SMILES: CNC(CCCNC(=N)NC(=O)C1CCCN1C(=O)C(CS)NC(C)=O)C(=O)NC(=N)NCCCC(N)C(=O)N1CCCC1C(=O)NC(Cc1ccc(O)cc1)C(=O)NC(C(=O)NC(CC(C)C)C(=O)O)C(C)(C)C ; "Cys-NTS20.8"]

See also, e.g., Yin, et al., Amino Acids, 49(8):1325-1335. doi: 10.1007/s00726-017-2430-5, which is specifically incorporated by reference herein in its entirety.

In another embodiment, the ligand is SR142948A or one or its derivatives, or an NTSR1 antagonist analog thereof (Moody, et al., Front Endocrinol, 9 (2018), Kling, ACS Chem Biol, 11, 869-75 (2016), Schaeffer, J Cardiovasc Pharmacol, 31, 545-50 (1998).

In some embodiments, the ligand has the following structure:

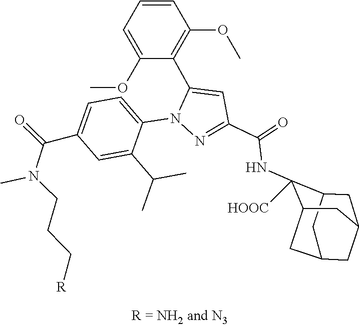

Other peptidomimetic and non-peptidic receptor agonists and antagonists are known in the art can be used as targeting ligands. See, e.g., Kleczkowska and Lipkowski, European Journal of Pharmacology, 716(1-3): 54-60 (2013), which is specifically incorporated by reference herein in its entirety.

### III. Pharmaceutical Compositions

Pharmaceutical compositions including 7-DHC or a derivative or analog thereof with or without a particle-based delivery platform are provided. Pharmaceutical compositions can be for, for example, administration by parenteral (e.g., intramuscular, intraperitoneal, intravenous (IV) or subcutaneous) injection or infusion.

In some embodiments, the pharmaceutical composition is a unit dosage containing an effective amount of a disclosed composition. In some embodiments, the unit dosage is in a unit dosage form for intravenous injection. In some embodiments, the unit dosage is in a unit dosage form for intratumoral injection.

In some embodiments, the compositions are administered systemically, for example, by intravenous or intraperitoneal administration, in an amount effective for delivery of the compositions to targeted cells.

In certain embodiments, the compositions are administered locally, for example, by subcutaneous injection, or injection directly into a site to be treated. In some embodiments, the compositions are injected or otherwise administered directly to one or more tumors. Typically, local injection causes an increased localized concentration of the compositions which is greater than that which can be achieved by systemic administration. In some embodiments, the compositions are delivered locally to the appropriate cells by using a catheter or syringe. Other means of delivering such compositions locally to cells include using infusion pumps (for example, from Alza Corporation, Palo Alto, Calif.) or incorporating the compositions into polymeric implants (see, for example, P. Johnson and J. G. Lloyd-Jones, eds., Drug Delivery Systems (Chichester, England: Ellis Horwood Ltd., 1987), which can effect a sustained release of the particles to the immediate area of the implant.

The compounds and particle-based formulations thereof can be provided to the cell either directly, such as by contacting it with the cell, or indirectly, such as through the action of any biological process. For example, the compounds and particle-based formulations thereof can be formulated in a physiologically acceptable carrier or vehicle, and injected into a tissue or fluid surrounding the cell.

### A. Formulations for Parenteral Administration

In a preferred embodiment the compositions are administered in an aqueous solution, by parenteral injection.

The formulation can be in the form of a suspension or emulsion. In general, pharmaceutical compositions are provided including an effective amount of a disclosed compound and optionally including pharmaceutically acceptable diluents, preservatives, solubilizers, emulsifiers, adjuvants and/or carriers. Such compositions can include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as polysorbate 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

### B. Other Formulations

The disclosed compounds alone or in a particle formulation can also be applied topically. Topical administration can include application to the lungs, nasal, oral (sublingual, buccal), vaginal, or rectal mucosa. In some embodiments, the compositions are administered in combination with transdermal or mucosal transport elements.

A wide range of mechanical devices designed for pulmonary delivery of therapeutic products can be used, including but not limited to, nebulizers, metered dose inhalers, and powder inhalers, all of which are familiar to those skilled in the art. Some specific examples of commercially available devices are the Ultravent® nebulizer (Mallinckrodt Inc., St. Louis, Mo.); the Acorn® II nebulizer (Marquest Medical Products, Englewood, Colo.); the Ventolin® metered dose inhaler (Glaxo Inc., Research Triangle Park, N.C.); and the Spinhaler® powder inhaler (Fisons Corp., Bedford, Mass.). Nektar, Alkermes and Mannkind all have inhalable insulin powder preparations approved or in clinical trials where the technology could be applied to the formulations described herein.

Oral formulations may be in the form of chewing gum, gel strips, tablets, capsules, or lozenges. Oral formulations may include excipients or other modifications to the particle which can confer enteric protection or enhanced delivery through the GI tract, including the intestinal epithelia and mucosa (see Samstein, et al., Biomaterials, 29(6):703-8 (2008).

Transdermal formulations may also be prepared. These will typically be ointments, lotions, sprays, or patches, all of which can be prepared using standard technology. Transdermal formulations can include penetration enhancers.

### IV. Methods of Use

### A. Methods of Treatment

Methods of use are provided. The experiments below demonstrate the use of an exemplary nanoparticle-based 7-DHC or a derivative or analog thereof in the treatment of cancer. Enhanced by nanoparticle delivery, the toxicity of 7-DHC is limited, allowing the therapeutic to be delivered systematically to tumors through the EPR effect and which may be enhanced by the addition of cell-specific targeting moiety. Subsequent radiation elevates oxidative stress in tumors, and results presented in the experiments below show that 7-DHC can react with radiation-induced reactive oxygen species and in turn promote lipid peroxidation, double-strand breaks, and mitochondrial damage in cancer cells. Thus, in some embodiments, 7-DHC or a derivative or analog thereof is administered in an effective amount to increase lipid peroxidation, double-strand breaks, and/or mitochondrial damage following treatment with radiation.

Results below including in vitro and in vivo testing against colon cancer, and in vitro testing against lung cancer. A nanoparticle radiosensitizer that can sensitize NSCLC cells to RT while causing minimal systemic toxicities is highly desirable. NSCLC accounts for 85% of all lung cancer cases, and is diagnosed in 234,030 persons in the US alone in 2018 (Jemal et al., Ca-Cancer J. Clin. 60, 277-300 (2010)). RT is the standard care for the majority of patients with locally advanced or local regional disease, and is a viable alternative to lobectomy and lymph node dissection for stage I patients (Baker et al., Radiat. Oncol. 11, 115 (2016)).

Thus, methods of treating a subject are provided. The methods typically include administering an effective amount of 7-DHC or a derivative or analog thereof to a subject in need thereof, typically in combination with radiotherapy. In preferred embodiments, the composition is delivered to the subject in using a particle-based delivery platform.

In the experiments below, mice were administered 10 mg/kg of 7-DHC-loaded PLGA nanoparticles (e.g., in 125 μl PBS) by intravenous delivery (e.g., tail vein injection). As further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired.

Generally, dosage levels of 0.001 to 100 mg/kg, for example, 0.1 mg/kg to 25 mg/kg, of body weight are administered to mammals. Generally, for local administration, dosage may be lower than for systemic administration. The dosage can be a daily dosage, or any other dosage regimen consistent with the disclosed methods. The timing of the administration of the composition will also depend on the formulation and/or route of administration used. The compound may be administered once daily, but may also be administered two, three or four times daily, or every other day, or once or twice per week. For example, the subject can be administered one or more treatments 1, 2, 3, 4, 5, 6, 7, 8, 9, 10, 11, 12, 13, 14, 15, 16, 17, 18, 19, 20, 21, 22, 23, or 24 hours, days, weeks, or months apart.

The subject can have one or more malignant or non-malignant tumors. In some embodiments, the subject has cancer. Thus, methods of treating cancer are also provided.

Typically, the composition is administered to the subject in combination with a radiation therapy. Although discussed herein primarily with reference to ionizing radiation therapy. In some embodiments, the radiotherapy does not include phototherapy, e.g., ultraviolet (UV) light therapy.

In some embodiments, the 7-DHC or a derivative or analog thereof is administered in an effective amount to enhance treatment of the tumor or cancer relative to administration of radiation alone, and/or administration of 7-DHC or a derivative or analog thereof alone. In some embodiments, 7-DHC or a derivative or analog thereof delivered with nanoparticles has reduced toxicity relative to free 7-DHC or the derivative or analog thereof.

Radiation with X-rays, gamma rays, protons, and neutrons may be used. Photodynamic therapy may also be used.

In preferred embodiments, the radiation is ionizing radiation. Ionizing radiation therapy (also referred to as radiotherapy and RT) is the medical use of ionizing radiation as part of cancer treatment to control malignant cells. Ionizing radiation is typically defined as radiation with enough energy to liberate an electron from the orbit of an atom, causing the atom to become charged or ionized. Ionizing radiation can be administered to a subject in need thereof as part of radiation therapy for the treatment for cancer. Examples of radiation therapy include, but are not limited to, external beam radiation therapy (EBRT or XRT) or teletherapy, brachytherapy or sealed source radiation therapy, and systemic radioisotope therapy or unsealed source radiotherapy. The radiation therapy can be administered to the subject externally (i.e., outside the body), or internally for example, brachytherapy which typically utilizes sealed radioactive sources placed in the area under treatment, and or systemic administration of radioisotopes by infusion or oral ingestion. Radiation therapy can include temporary or permanent placement of radioactive sources on or within the subject. Another example of radiation therapy is particle therapy which typically includes external beam radiation therapy where the particles are protons or heavier ions.

Radiation therapy works by damaging the DNA of dividing cells, e.g., cancer cells. This DNA damage is caused by one of two types of energy, photon or charged particle. This damage is either direct or indirect. Indirect ionization happens as a result of the ionization of water, forming free radicals, notably hydroxyl radicals, which then damage the DNA. For example, most of the radiation effect caused by photon therapy is through free radicals. One of the major limitations of photon radiotherapy is that the cells of solid tumors become deficient in oxygen, and tumor cells in a hypoxic environment may be as much as 2 to 3 times more resistant to radiation damage than those in a normal oxygen environment.

Direct damage to cancer cell DNA occurs through high-LET (linear energy transfer) charged particles such as proton, boron, carbon or neon ions. This damage is independent of tumor oxygen supply because these particles act mostly via direct energy transfer usually causing double-stranded DNA breaks. Due to their relatively large mass, protons and other charged particles have little lateral side scatter in the tissue; the beam does not broaden much, stays focused on the tumor shape and delivers small dose side-effects to surrounding tissue.

The amount of radiation used in photon radiation therapy is measured in Gray (Gy), and varies depending on the type and stage of cancer being treated. For curative cases, the typical dose for a solid epithelial tumor ranges from 60 to 80 Gy, while lymphomas are treated with 20 to 40 Gy. Post operative (adjuvant) doses are typically around 45-60 Gy in 1.8-2 Gy fractions (for breast, head, and neck cancers). Many other factors are considered by radiation oncologists when selecting a dose, including whether the patient is receiving chemotherapy, patient co-morbidities, whether radiation therapy is being administered before or after surgery, and the degree of success of surgery.

The response of a cancer to radiation is described by its radiosensitivity. Highly radiosensitive cancer cells are rapidly killed by modest doses of radiation. These include leukemias, most lymphomas and germ cell tumors. The majority of epithelial cancers are only moderately radiosensitive, and require a significantly higher dose of radiation (60-70 Gy) to achieve a radical cure. Some types of cancer are notably radioresistant, that is, much higher doses are required to produce a radical cure than may be safe in clinical practice. Renal cell cancer and melanoma are generally considered to be radioresistant.

In some embodiments, the compositions and methods reduce the dose of radiation required to induce a curative or preventative effect. For example, the disclosed compounds can increase a cancer's radiosensitivity. Effective doses of radiation therapy may be toxic for certain cancers. In some embodiments, the compounds decrease the required effective dose of radiation needed to treat a cancer, thereby reducing toxicity of the effective dose of radiation.

In other embodiments, the disclosed compounds may be used with normal doses of drug or radiation to increase efficacy. For example, the compounds may be used to potentiate a radiation therapy for a cancer that is radiation resistant.

The response of a tumor to radiotherapy is also related to its size. For complex reasons, very large tumors respond less well to radiation than smaller tumors or microscopic disease. Various strategies are used to overcome this effect. The most common technique is surgical resection prior to radiotherapy. This is most commonly seen in the treatment of breast cancer with wide local excision or mastectomy followed by adjuvant radiotherapy. Another method is to shrink the tumor with neoadjuvant chemotherapy prior to radical radiotherapy. In some embodiments, the disclosed methods allow for treatment of tumors that are larger than can be treated by a normal dose of radiation.

A third technique is to enhance the radiosensitivity of the cancer by giving certain drugs during a course of radiotherapy. The disclosed compositions can serve this third function. In these embodiments, the compound increases the cell's sensitivity to the radiotherapy, for example, by at least 10%, 15%, 20%, 25%, 30%, 35%, 40%, 45%, 50%. Moreover, the compound can be combined with one or more additional radiosensitizers. Examples of known radiosensitizers include cisplatin, gemcitabine, 5-fluorouracil, pentoxifylline, vinorelbine, PARP inhibitors, histone deacetylase inhibitors, and proteasome inhibitors, and others mentioned elsewhere herein.

Radiation therapy can be administered to a subject in combination with surgery, chemotherapy, hormone therapy, immunotherapy, or combination thereof. For example, intraoperative radiation therapy or (IORT) is delivered immediately after surgical removal of a cancer. This method has been employed in breast cancer (TARGeted Introperative radiation therapy or TARGIT), brain tumors and rectal cancers.

Radiotherapy also has several applications in non-malignant conditions, such as the treatment of trigeminal neuralgia, severe thyroid eye disease, pterygium, pigmented villonodular synovitis, prevention of keloid scar growth, and prevention of heterotopic ossification. Thus, in some embodiments, the compositions and methods are used to increase radiosensitivity for a non-malignant condition.

Typically, the prodrug composition is administered before, e.g., minutes, hours, or days before, a radiation therapy. For example, in exemplary embodiments, a dose of radiation is administered 1 hour to 48 hours, or 1 hour to 24 hours, or 1 hour to 12 hours, or 1 hour to 6 hours, or 2 hours to 6 hours, or 1, 2, 3, 4, or 5 hours after administration of the pharmaceutical composition. In some embodiments, 1, 2, 3, 4, or 5 rounds are radiation are administered after each single dose of the composition. In some embodiments, the composition is administered one or more times for each round of radiation. In some embodiments, each cycle of radiation is preceded by a cycle of the composition. For example, in particular embodiments, 1, 2, 3, 4, 5, 6, 7, 8, 9, 10, or more rounds of administration of the pharmaceutical composition followed by administration of the dose of radiation are carried out in tandem.

In some embodiments, the disclosed compositions and methods are more effective, less toxic, or combination thereof relative to a specific concurrent or sequential chemo-radiotherapy (CRT).

### B. Cancers to be Treated

The compositions and methods described herein are useful for treating subjects having benign or malignant tumors by delaying or inhibiting the growth of a tumor in a subject, reducing the growth or size of the tumor, inhibiting or reducing metastasis of the tumor, and/or inhibiting or reducing symptoms associated with tumor development or growth. The treatment is also useful for reducing overproliferation of non-cancerous tissues such as endometriosis, restenosis, and scarring (fibrosis).

Malignant tumors which may be treated are classified according to the embryonic origin of the tissue from which the tumor is derived. Carcinomas are tumors arising from endodermal or ectodermal tissues such as skin or the epithelial lining of internal organs and glands. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

The types of cancer that can be treated with the provided compositions and methods include, but are not limited to, cancers such as vascular cancer such as multiple myeloma, adenocarcinomas and sarcomas, of bone, bladder, brain, breast, cervical, cob-rectal, esophageal, kidney, liver, lung, nasopharyngeal, pancreatic, prostate, skin, stomach, and uterine. In some embodiments, the disclosed compositions are used to treat multiple cancer types concurrently. The compositions can also be used to treat metastases or tumors at multiple locations.

In some embodiments, the cancer is a highly radiosensitive, moderately radiosensitive cancer, or radioinsensitive (i.e., low radiosensitive cancer). Highly radiosensitive cancer cells are rapidly killed by modest doses of radiation. Tissues rich in actively dividing cells generally show high sensitivity to radiation, whereas those with few such cells have low radiosensitivity (Hayabuchi, JMAJ, 47(2): 79-83 (2004)). For example, genital glands such as the testis and ovary, lymphatic tissue, fetal tissue, and fetus-like blast cell tissue are highly radiosensitive. Tissues with low radiosensitivity include adult bone, fatty tissue, muscle, and large vessels. Because the radiosensitivity of a tumor reflects the sensitivity of the tissue from which it has arisen, malignant lymphomas, which originate in lymphatic tissue, and seminomas, which originate in the testis, have high sensitivity to radiation. In contrast, osteogenic sarcomas and liposarcomas demonstrate low radiosensitivity.

Epithelial tumors and cancers, are considered to have moderate radiosensitivity. Such cancers can require a significantly higher dose of radiation (60-70 Gy) to achieve a cure. Among these tumors, undifferentiated carcinoma and small cell carcinoma have relatively high radiosensitivity, followed by squamous cell carcinoma. The radiosensitivity of adenocarcinoma is generally lower than that of other types of epithelial tumors. In light of this, head and neck cancer, esophageal cancer, uterine cervical cancer, and skin cancer, among which squamous cell carcinoma is common, seem to be good indications for radiotherapy.

However, even among squamous cell carcinomas of the esophagus, some are highly radiosensitive but others are not. Radiosensitivity can depend not only on the histologic type of the tumor but also on other factors, for example, the oxygen concentration in the tumor and the mitotic cycle of tumor cells.

Renal cell cancer and melanoma are generally considered to be radioresistant.

In some embodiments, the cancer is colorectal cancer. Colorectal cancer is the third most prevalent cancer type among both men and women in the United States, (Restivo, et al., Oncol. Res. Treat., 43, 146-152 (2020)) and is the second leading cause of cancer-related death worldwide. Radiation therapy is a mainstay treatment option (Gustaysson, et al., Clin. Colorectal Cancer, 14, 1-10 (2015)) but may induce morbidities including rectal irritation, scarring, fibrosis, and sexual issues, limiting its lifetime dose to ˜60 Gy (Xu, et al., J. Am. Chem. Soc., 132, 2222-2232 (2010)). The importance of enhancing radiotherapy efficacy with a radiation-activatable radiosensitizer cannot be overemphasized.

In some embodiments, the cancer is a lung cancer, for example, a non-small cell lung cancer (NSCLC). In other embodiments the cancer is a head and neck, breast, or colon cancer.

In some embodiments, particularly those wherein nanoparticles feature an NTSR1 targeting signal, the cancer has upregulated NTSR1. In some embodiments, the cancer in which NTSR1 is upregulated is a lung cancer, for example, a non-small cell lung cancer (NSCLC), a head and neck, breast, or colon cancer.

The disclosed invention can be further understood by the following numbered paragraphs:
1. A method of treating a subject in need thereof including administering the subject a pharmaceutical composition including
an effective amount of 7-dehydrocholesterol (7-DHC) or analog or derivative thereof, and
one or more doses of radiation therapy, optionally non-ultraviolet radiation therapy.
2. The method of paragraph 1, wherein the subject has cancer.
3. A method of treating cancer including administering a subject with cancer a pharmaceutical composition including
an effective amount of 7-dehydrocholesterol (7-DHC) or analog or derivative thereof, and
one or more doses of radiation therapy, optionally non-ultraviolet radiation therapy.
4. The method of any one of paragraphs 1-3, wherein the radiation therapy includes X-rays, gamma rays, protons, or neutrons; and/or
wherein the radiation is photodynamic therapy or ionizing radiation therapy;
optionally wherein the radiation therapy is administered in an effective amount to induce the chemical reaction according to Scheme 1 with 7-DHC or the corresponding chemical reaction in the analog or derivative thereof.
5. The method of any one of paragraphs 1-4, wherein the 7-DHC or analog or derivative thereof is encapsulated or incorporated into or otherwise forms part of nanoparticles.
6. The method of paragraph 5, wherein the nanoparticles are polymeric nanoparticles, lipoprotein-like particles, liposomes, inorganic nanoparticles, or a combination thereof.
7. The method of paragraph 6, wherein the nanoparticles are polymeric nanoparticles including one or more amphiphilic, hydrophobic, and/or hydrophilic polymers.
8. The method of paragraph 7, wherein the nanoparticle includes one or more hydrophobic polymers.
9. The method of paragraph 8, wherein one or more of hydrophobic polymers is polyester.
10. The method of paragraph 9, wherein the polyester or polyesters are selected from poly(lactic acid-co-glycolic acid)s, poly(lactic acid), poly(glycolic acid).
11. The method of any one of paragraphs 5-10, wherein the nanoparticles include poly(lactic acid-co-glycolic acid) (PLGA).
12. The method of any one of paragraphs 5-10, wherein the nanoparticles are lipoprotein or lipoprotein-like nanoparticles.
13. The method of paragraph 12, wherein the nanoparticles are low-density lipoprotein (LDL) or LDL-like nanoparticles.
14. The method of paragraphs 12 or 13, wherein the nanoparticles include one or more triglyceride(s) and one or more phospholipid(s).
15. The method of paragraph 14, wherein the triglyceride(s) includes oleic acid (OA) and/or triolein (TO).
16. The method of paragraphs 14 and 15, wherein the phospholipid(s) include phosphatidyl choline (PC).
17. The method of any one of paragraphs 12-16, wherein the lipoprotein or lipoprotein-like nanoparticles are synthetic.
18. The method of any one of paragraphs 12-16, wherein the lipoprotein or lipoprotein-like nanoparticles is natural lipoprotein isolated from a human subject, optionally wherein the lipoprotein is natural LDL.
19. The method of any one of paragraphs 12-18, wherein the lipoprotein or lipoprotein-like nanoparticles are free from protein.
20. The method of any one of paragraphs 5-19, wherein the nanoparticles have a size suitable for delivery of the 7-DHC to tumor microenvironments by enhanced permeability and retention.
21. The method of any one of paragraphs 5-19, wherein the nanoparticle have a size of about 10 nm to about 300 nm, or about 50 nm to about 150 nm.
22. The method of any one of paragraphs 5-19, wherein the nanoparticles further include a targeting agent coupled thereto.
23. The method of paragraph 22, wherein the targeting agent targets NTSR1.
24. The method of paragraph 23, wherein the targeting agent is an agonist or antagonist for NTSR1.
25. The method of any one of paragraphs 22-24, wherein the targeting agent is NTS or a variant thereof.
26. The method of paragraph 25, wherein targeting agent is NTSₘᵤₜ.
27. The method of any one of paragraphs 24-26, wherein the targeting agent is SR142948A or NTS_{20.8.}
28. The method of any one of paragraphs 22-27, wherein the target agent including or consisting of the sequence of any one of SEQ ID NOS:1-7.
29. The method of any one of paragraphs 1-28, wherein the composition enhances the treatment of the cancer compared to administration of the radiation alone.
30. The method of any one of paragraphs 1-29, wherein the cancer is a radiosensitive cancer.
31. The method of any one of paragraphs 1-29, wherein the cancer is a radioresistant cancer.
32. The method of any one of paragraphs 1-31, wherein the cancer a vascular, bone, muscle, bladder, brain, breast, cervical, cob-rectal, esophageal, kidney, liver, lung, nasopharyngeal, pancreatic, prostate, skin, stomach, uterine, or germ cell.
33. The method of any one of paragraphs 1-32, wherein the cancer is an epithelial cancer.
34. The method of any one of paragraphs 1-32, wherein the cancer is a non-small cell lung cancer (NSCLC).
35. The method of any one of paragraphs 1-34, wherein the same dose of radiation is more effective than when administered in the absence of the pharmaceutical composition, a lower dose of radiation has the same effectiveness as a higher dose when administered in the absence of the pharmaceutical composition, or a combination thereof.
36. The method any one of paragraphs 1-35, wherein a dose of radiation is administered after administration of the pharmaceutical composition.
37. The method of any one of paragraphs 1-36, wherein the dose of radiation is administered 1 to 48 hours, or 1 to 24 hours, or 1 to 12 hours, or 1 to 6 hours, or 2 to 6 hours, or 1, 2, 3, 4, or 5 hours after administration of the pharmaceutical composition.
38. The method of any one of paragraphs 1-37, including 1, 2, 3, 4, 5, 6, 7, 8, 9, 10, or more rounds of administration of the pharmaceutical composition followed by administration of the dose of radiation, optionally wherein the dose of radiation is administered after one, some, or all of the rounds of administration of the pharmaceutical composition.
39. The method of any one of paragraphs 1-38, wherein the 7-DHC or analog or derivative thereof is 7-DHC.
40. The method of any one of paragraphs 1-38, wherein the 7-DHC or analog or derivative thereof is an analog or derivative thereof is 7 DHC.
41. The method of paragraph 40, wherein the analog or derivative thereof is a compound of any of:

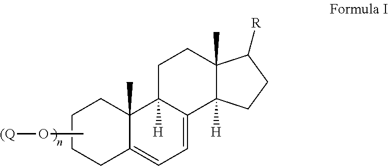

wherein n is an integer value selected from 1, 2, 3, or 4;
wherein R is a linear or branched alkyl or heteroalkyl group, alkenyl or heteroalkenyl, or alkynyl or heteroalkynyl group, where each may be optionally substituted by one or more substituents; and
wherein Q is a hydrogen, a linear or branched alkyl or heteroalkyl group, alkenyl or heteroalkenyl, or alkynyl or heteroalkynyl group, a cycloalkyl group, a heterocycloalkyl, an aryl, a heteroaryl group, acyl group, a sulfonate group, or a siloxy group, where each may be optionally substituted with one or more substituents;
or is a salt thereof;

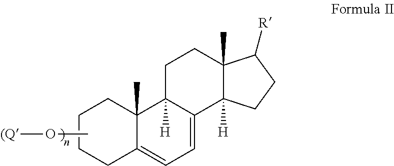

wherein n is an integer value selected from 1, 2, 3, or 4;
wherein R′ is a linear or branched alkyl or heteroalkyl group, alkenyl or heteroalkenyl, or alkynyl or heteroalkynyl group, where each may be optionally substituted by one or more substituents; and
wherein Q′ is a hydrophilic polymer or a hydrophilic saccharide; or is a salt thereof;

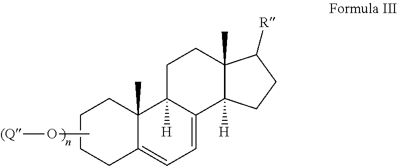

wherein n is an integer value selected from 1, 2, 3, or 4;
wherein R″ is a linear or branched alkyl or heteroalkyl group, alkenyl or heteroalkenyl, or alkynyl or heteroalkynyl group, where each may be optionally substituted by one or more substituents; and
wherein Q″ is a therapeutic agent;
or is a salt thereof;

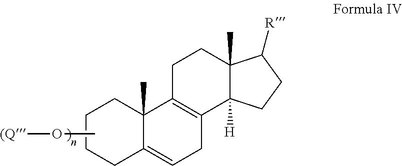

wherein n is an integer value selected from 1, 2, 3, or 4;
wherein R″′ is a linear or branched alkyl or heteroalkyl group, alkenyl or heteroalkenyl, or alkynyl or heteroalkynyl group, where each may be optionally substituted by one or more substituents; and
wherein Q″′ is a hydrogen, a linear or branched alkyl or heteroalkyl group, alkenyl or heteroalkenyl, or alkynyl or heteroalkynyl group, a cycloalkyl group, a heterocycloalkyl, an aryl, a heteroaryl group, acyl group, a sulfonate group, or a siloxy group, where each may be optionally substituted with one or more substituents;
or is a salt thereof;

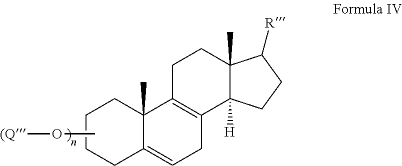

wherein n is an integer value selected from 1, 2, 3, or 4;
wherein R″″ is a linear or branched alkyl or heteroalkyl group, alkenyl or heteroalkenyl, or alkynyl or heteroalkynyl group, where each may be optionally substituted by one or more substituents; and
wherein Q″″ is a hydrophilic polymer or a hydrophilic saccharide; or is a salt thereof; or

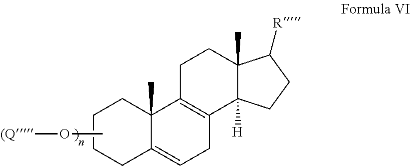

wherein n is an integer value selected from 1, 2, 3, or 4;
wherein R″″′ is a linear or branched alkyl or heteroalkyl group, alkenyl or heteroalkenyl, or alkynyl or heteroalkynyl group, where each may be optionally substituted by one or more substituents; and
wherein Q″″′ is a therapeutic agent;
or is a salt thereof.
42. The method of paragraphs 40 and 41 wherein a plurality of the analog or derivative thereof can self-assemble into nanoparticles.
43. A compound of any of Formulae I-VI.
44. A nanoparticle including a compound of any of Formulae I-VI.
45. The compound or nanoparticle of paragraphs 43 or 44 further including a therapeutic agent conjugated thereto.
46. The compound or nanoparticle of paragraph 45, wherein the therapeutic agent is a small molecule chemotherapeutic agent.
47. The nanoparticle of any one of paragraphs 44-46 formed by self-assembly of a plurality of the compounds of Formulae II or V alone or in combination with one or more other polymers, lipids, proteins, or saccharides.
48. A pharmaceutical composition including the compound or nanoparticle of any one of paragraphs 43-47.
49. The pharmaceutical composition of any one of paragraphs 1-42.
50. The pharmaceutical composition of paragraph 49 including the nanoparticles according to any of paragraphs 3-28.
51. A compound, composition, or method as describe herein, including, but not limited to the text and figures.

### EXAMPLES

### Example 1: Synthesis and Characterization of 7-DHC@PLGA NPs

### Materials and Methods

### 7-DHC@PLGA-PEG Nanoparticle Synthesis:

7-DHC@PLGA-PEG nanoparticles (7-DHC@PLGA NPs) were synthesized by a nanoprecipitation method following previously published protocols. Briefly, 1.5 mg of 7-DHC and 5 mg of PLGA-b-PEG-OH were dissolved in 1 mL CH3CN, and the solution was added to 10 mL of Mil-Q-H2O. The reaction was stirred at room temperature for 2 hours, and the resulting solution was purified by multiple rounds of centrifugation at 2,800 RPM for 10 minutes. The final product was stored in PBS at 4° C. for further experiments and storage for up to 1 week.

### Physicochemical Characterization of 7-DHC@PLGA NPs:

Transmission electron microscopy (TEM) was carried out on a FEI TECNAI 20 transmission electron microscope at 200 kV. The zeta potential and size distribution measurements were carried out on a Malvern Zetasizer Nano ZS system (Zeta potential −13.9 mV, DLS 90.0 nm). 7-DHC loading and release studies were carried out using the absorption of 7-DHC at 282 nm on a BioTek Synergy MX multi-mode microplate reader. The absorbance of 7-DHC was subtracted from the water background of the PLGA polymer at 260 nm to account for any potential overlap in absorbance readings. The corrected absorbance was then compared to an experimentally generated standard curve to extrapolate the drug loading and encapsulation efficiency of 7-DHC within the polymer NP.

### 7-DHC Release:

The 7-DHC@PLGA NPs were incubated in 2 mL PBS at different pH values (pH=5.5, 6.5, and 7.2) to test the stability of the nanoparticle and the drug release. The samples were kept in an incubating shaker at 37° C. in a 10 k MWCO dialysis tube. At each time point (0, 0.25, 0.5, 1, 2, 4, 8, 12 and 24, 48 hours), aliquots of the samples were collected and centrifugated using a micro filter unit (MWCO: 10 k; Amicon® Cat #UFC800308). The solution's absorbance was analyzed using BioTek Synergy MX multi-mode microplate reader. It was once again compared to an experimentally generated standard curve to determine the amount of free 7-DHC in the lower portion of the dialysis tube. This absorbance corresponded to the quantity of drug released from the nanoparticle at the given time point.

### Statistical Analysis:

The means and standard errors were calculated from at least three repeated groups in all the experiments. Statistical significance between groups was determined with the Student's t test where P<0.05 was considered to be statistically significant between two groups. *P<0.05; **P<0.01; ***P<0.001; ****P<0.0001; ns, no significant difference.

### Results

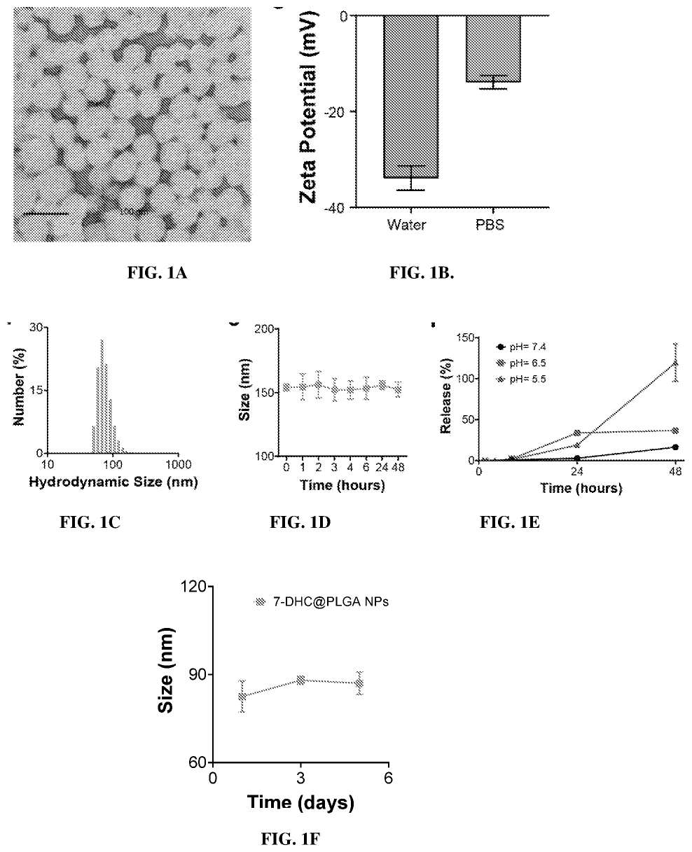

7-DHC@PLGA NPs were prepared through nanoprecipitation. Uv-vis spectroscopy found that the loading capacity (LC %) is 7.1% and the encapsulation efficiency (EE %) is 21.9%. Transmission electron microscopy (TEM) with negative staining revealed that 7-DHC@PLGA NPs were 88.0±3.2 nm in diameter (FIG. 1A). Dynamic light scattering (DLS) recorded a hydrodynamic size of ˜90 nm and a polydispersity index (PDI) of 0.100 (FIG. 1B). The surface zeta potential of 7-DHC@PLGA NPs was −13.9 mV in PBS (FIG. 1C). The nanoparticles were well dispersed in aqueous solutions, showing minimal size change over 5 days (FIGS. 1D and 1E). 7-DHC release was analyzed in buffer solutions with pH 5.5, 6.5 and 7.4, respectively. Relative to neutral pH, 7-DHC release accelerated at pH 5.5 and 6.5 (FIG. 1F), which correspond to the pH of lysosomes or late endosomes (Korade, et al., J Inherit Metab Dis., 36, 113-122 (2013)) and acidic tumor micromovement, (Korade, et al., J. Lipid Res., 51, 3259-3269 (2010)) respectively.

### Example 2: DHC@PLGA NPs Promote Radiation-Induced Decrease in Cell Viability and Proliferation

### Materials and Methods

### Cell Culturing:

CT26, a murine colorectal carcinoma cell line, was cultured following the protocol provided by the ATCC. A complete growth medium was prepared by adding 50 mL fetal bovine serum (FBS; Atlanta Biologicals, Cat #S11150) and 5 mL penicillin-streptomycin (Corning Cat #30-002-CI) to 450 mL of RPMI 1640 medium (Corning, Cat #10-104-CV). The cells were sub-cultured every three days and stored in a Thermo Scientific Heracell 150i incubator at 37° C. A day before the experiment, the cells were washed with PBS and collected by trypsinization (37° C., 2 min) followed by neutralization with cell culture medium and centrifugation (1200 rpm, 5 min). The supernatant was removed, and cells were dispersed in new cell culture medium. The cell density was counted using a hemocytometer (Hausser Scientific, Cat #3200) to seed the desired number of cells on the experimental plate(s).

### Cytotoxicity Studies:

The cell viability was studied with CT26 cells using the standard MTT assays. Briefly, CT26 cells (10,000 cells per well) were seeded on clear 96-well plates (Corning Costar, Cat #3599). When the cells adhered to the bottom, 0-100 μg/mL of 7-DHC@PLGA NPs were added to the cells and incubated for 24 h. 20 μL of 10 mg/mL 3-(4,5-Dimethylthiazolyl-2)-2,5-diphenyltetrazolium bromide (MTT) solution was added into each well. After 4 h incubation at 37° C. and 5% CO₂, the solution was discarded from each well, and 100 μL of DMSO was added to each well resulting in purple suspensions. The absorbance at 570 nm was measured using a BioTek Synergy MX multi-mode microplate reader.

### Intracellular ATP Analysis:

CT26 cells at density of 10,000 cells (100 μL) per well were plated in white 96-well plates (Corning Costar, Cat #3610) and incubated at 37° C. for 24 h. 7-DHC@PLGA or 7-DHC@PLGA NPs+IR were suspended in cell culture medium. The cells were treated with 7-DHC@PLGA (5 μg/mL), 7-DHC@PLGA NPs+IR (5 μg/mL), IR alone, or PBS and incubated for another 24 h followed by 5 Gy irradiation in the IR respective groups. The IR groups were then incubated at 37° C. for 30 minutes, followed by incubation at room temperature for 30 minutes. Subsequently, the supernatant from each well was removed completely. Then, 100 uL ATPLite (PerkinElmer Cat #016943) solution was added following the manufacturers protocol. The plate was then shaken in the dark for 10 minutes, and the luminescence was read using a microplate reader (Synergy Mx, BioTeK). The result was compared to the standard curve prepared according to the manufacture's protocol.

### Caspase 3 Activity:

CT26 cells were seeded in a black 96-well-plate (Corning Costar, Cat #3614) at a cell density of 8,000 cells per well and incubated overnight. The cells were incubated with PBS or 7-DHC@PLGA NPs (5 μg/mL) for 4 h followed by 5 Gy irradiation. The cells were incubated another 24 h, and the Caspase 3 activity was evaluated using FAM-FLICA® Caspase-3/7 kit (Immunochemistry, Cat #94) following the manufacturer's protocol. Briefly, the medium was replaced with 96.7 μL of cell culture medium and 3.3 μL of FAM-FLICA working solution. After 1 h of incubation at 37° C., FAM-FLICA working solution containing cell culture medium was replaced with 1× Apoptosis Wash Buffer three times to remove any unbound FAM-FLICA. The wash buffer was replaced with 100 μL PBS. The caspase 3 activity was evaluated by measuring the fluorescence (ex/em 488 nm/530 nm) with a microplate reader (Synergy Mx, BioTeK).

### Clonogenic Assay:

To evaluate the therapeutic effect of 7-DHC@PLGA, a clonogenic assay was performed. Briefly, 1×10⁶ CT26 cells in 1.5 mL cell culture medium were seeded in a 35 mm cell culture dish (Corning, Cat #430165) and stored in a 37° C. incubator overnight. Then, the cell culture medium was refreshed with 7-DHC@PLGA NPs at 5 μg/mL or PBS for 24 h. Then, the cells were collected and 100-10,000 cells were seeded on a 100 mm cell culture plate (Corning, Cat #353003). The cells were irradiated with the corresponding dose using a 300 kV X-ray generator, and then cultured for 14 days. The colonies were stained with crystal violet, counted, and a survival fraction was evaluated by the linear quadratic (LQ) model: S=e^{-(αD+βD}^{²}⁾

### Results

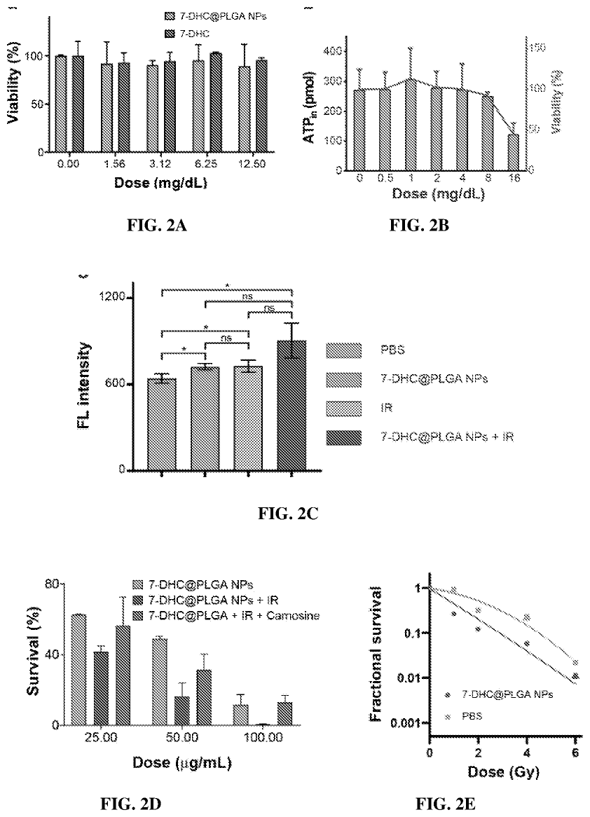

The cytotoxicity of 7-DHC and 7-DHC@PLGA NPs was investigated with CT26 cells by MTT assay. Neither 7-DHC nor 7-DHC@PLGA NPs caused significant viability drop when 7-DHC concentration was below 12.5 μg/mL (FIG. 2A). 7-DHC@PLGA NPs showed a slight increase in toxicity compared to 7-DHC (IC₅₀ 52.0 μg/mL vs. 71.4 μg/mL). This is attributed to the fact that some 7-DHC@PLGA NPs were directly taken up by cancer cells, which was confirmed by confocal microscopy. Cytotoxicity was tested by ATP bioluminescence viability assay and observed similar results (FIG. 2B).

Next, whether 7-DHC@PLGA NPs promote cell death induced by ionizing radiation (IR) was investigated. 7-DHC@PLGA NPs (5 μg/mL) were incubated with CT26 cells for 24 h, followed by IR (320 KV, 5 Gy). Notably, 7-DHC@PLGA NPs alone caused no cytotoxicity at this dose (FIG. 2C). Control groups received IR or PBS only. Compared to cells treated with only IR, cells treated with 7-DHC@PLGA NPs plus IR demonstrated a 24.3% increase in caspase-3 activity (FIG. 2C). This coincides with a viability drop of more than 83.5%, as indicated by MTT results (FIG. 2D). The radiosensitizing effects of 7-DHC@PLGA NPs was also investigated using clonogenic assay. Relative to IR alone, 7-DHC@PLGA NPs+IR significantly reduced the number of colonies formed at all tested radiation doses. The survival fraction was fit relative to untreated control into the linear-quadratic model, S=e^{-(αD+βD}^{²}⁾, where D is the radiation dose and a and b are fitting coefficients. It was determined that survival fraction at dose 2 was 0.209 for 7-DHC@PLGA NPs+IR, compared to 0.530 for IR alone. This represents a dose modifying factor of 2.536 (FIG. 2E, Table 2).

TABLE 2
Survival fractions relative to the PBS control
fit into the linear quadratic (LQ) model.

| | PBS | 7-DHC@PLGA NPs |
|---|---|---|
| a | 0.166 | 0.767 |
| b | 0.076 | 0.008 |
| a/b | 2.200 | 98.496 |
| SF2 | 0.530 | 0.209 |
| DMF | — | 2.536 |

Overall, while 7-DHC@PLGA NPs have low toxicity, their presence facilitates a reduction in viability and proliferation among cancer cells.

### Example 3: 7-DHC@PLGA NPs Enhance Lipid Peroxidation

### Materials and Methods

### Lipid Peroxidation Assay:

Image-iT Lipid Peroxidation Kit (Abcam, Cat #ab118970) was used to test lipid damage. Cells were seeded in a black 96-well-plate (Corning Costar, Cat #3614, 8000 cells per well) and stored in an incubator overnight. The cell culture medium was refreshed with 200 μL culture medium containing either 7-DHC@PLGA NPs (5 μg/mL), PBS (30 μg/mL), or 7-DHC@PLGA NPs+IR (5 μg/mL) and incubated at 37° C. After 24 h, excess particles were removed and fresh medium was added. The cells were irradiated by X-ray (5 Gy), and incubated for another 24 h. After removing the cell culture medium, 200 μL of image-iT Lipid peroxidation sensor (30 μM) dispersed in cell culture medium was added to the cells. After incubation for 30 minutes at 37° C., the medium was removed, and the cells were washed with PBS three times. The fluorescence was read at two separate wavelengths (ex/em at 581/591 nm and ex/em at 488/510 nm) using a microplate reader (Synergy Mx, BioTeK). The ratio of the emission fluorescence intensities at 590 nm to 510 nm was evaluated to analyze lipid peroxidation in cells.

### TBARS Assay:

TBARS Assay Kit (Cayman Chemical, Cat #100009055) was used to test for the production of MDA. Cells were seeded 100 mm cell culture dishes (Corning, Cat #353003, 10⁶ cells per dish) and stored in an incubator overnight. The cell culture medium was refreshed with 10 mL culture medium containing either 7-DHC@PLGA NPs (5 μg/mL), PBS (30 μg/mL), or 7-DHC@PLGA NPs+IR (5 μg/mL) and incubated at 37° C. After 24 h, excess particles were removed, and fresh medium was added. The cells were irradiated by X-ray (5 Gy), and incubated for another 24 h. The cells were then collected using a cell scraper, and each treatment group was suspended in 1 mL PBS. The cells were lysed on ice using a probe sonicator. The assay was then performed following the manufacturers protocol. Briefly, 100 μl of either the sample or the standard was added to a 15 mL centrifuge tube. 100 μl SDS solution was added to each tube, as well as 4 mL of the provided color reagent. All tubes were then boiled for 1 h, then placed in an ice bath to stop the reaction. Each sample was then centrifuged at 4° C. for 10 minutes at 1600×g. Each sample or standard was then loaded onto a clear 96-well plate (Corning, Cat #3599) in triplicate and the absorbance at 530 nm was read using a microplate reader (Synergy Mx, BioTeK). To determine the MDA concentrations, the absorbance readings for each experimental group were averaged and compared to an experimentally generated standard curve.

### LC/MS to Analyze Autooxidation and Lipid Peroxidation:

Samples were generated by mixing solutions of 10 mg/mL 7-DHC, or 5 mg/mL DHCEO in 5 mL solvent containing 4 mL CH3CN+1 mL Mil-Q-H₂O (or 1 mL H₂O₂). Samples were treated with either PBS or 5 Gy IR followed by incubation for 24 h. The lipid sample was prepared by using 16:0-18:2 PC mixed with 7-DHC at a 3:1 mole ratio in 2:1 v/v of CHCl₃:MeOH solution. The solvent was then rotor evaporated and the mixture was rehydrated in 1 mL Mil-Q-H₂O. Samples were subjected to LC/MS analysis. Liquid chromatography was performed on an Applied Biosystems 140 B solvent delivery system using water with 0.1% formic acid as solvent A and acetonitrile as solvent B. The linear solvent gradient was from 70% B to 95% B over 20 minutes at a flow rate of 50 μl/min. A Thermo Hypersil-Keystone 1×150 mm Biobasic-4 column with 5 μm particle size and 300 A pore size was used. The effluent was directed into a Bruker Daltonics Esquire 3000 plus ion trap mass spectrometer equipped with an atmospheric pressure chemical ionization (electrospray ionization for the lipid sample) source. The instrument was scanned in enhanced mode from 340-500 m/z with the capillary at 4 KV. The dry as temperature was held at 300 degrees C. at a flow rate of 41/min nitrogen. The nebulizer was set to 15 PSI of nitrogen. The vaporizer temperature was held at 380 degrees C.

### Results

Next investigated were the mechanisms behind the radiosensitizing effects of 7-DHC. Solutions including 7-DHC only or a mixture of 7-DHC and 1-palmitoyl-2-linoleoyl-sn-glycero-3-phosphocholine (16:0-18:2 PC). 16:0-18:2 PC contains a polyunsaturated fatty acid (PUFA) tail; it forms liposomes with 7-DHC, recapitulating cell membranes where sterols are inserted into a lipid layer formed of mainly phospholipids (Lamberson, et al., J. Org. Chem., 78, 3511-3524 (2013)). LC/MS found that after radiation (320 kV, 5 Gy), 7-DHC was converted to oxysterols including 3β,5α-dihydroxycholest-7-en-6-one (DHCEO). It is postulated that 7-DHC quickly reacts with radiation-induced radicals, for instance by losing a hydrogen atom to ROS (Scheme 1).

Scheme 1—Proposed mechanisms for 7-DHC-enhanced lipid peroxidation under radiation. 7-DHC easily loses one H· to radiation-induced ROS. The resulting radical can quickly react with O₂ to form a peroxide, and then goes through autoxidation to form oxysterols such as DHCEO. Alternatively, the radical reacts with PUFAs in cell membranes, triggering lipid peroxidation. The products of lipid oxidation include reactive aldehydes and ketones such as MDA.

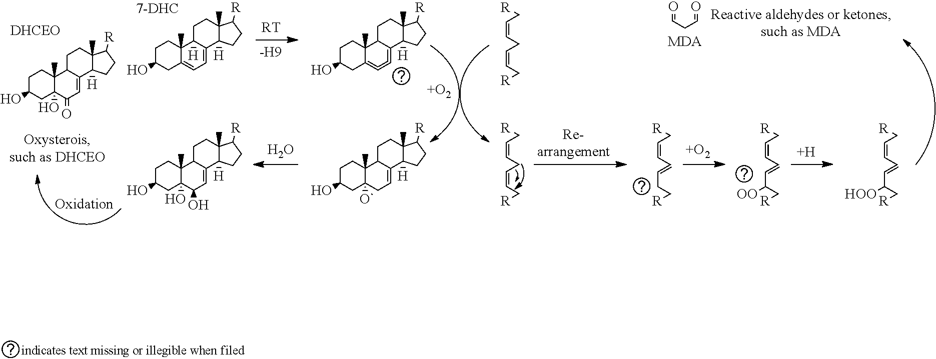

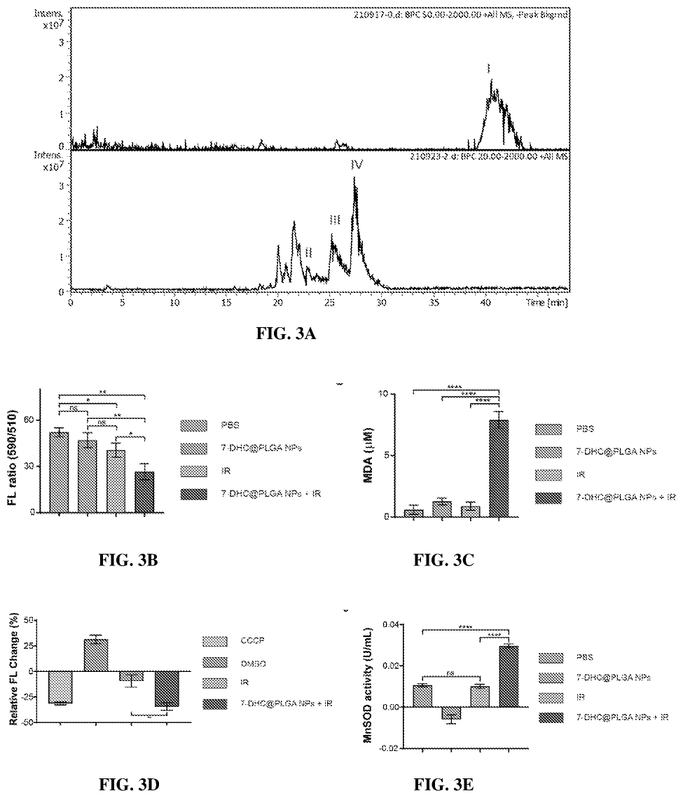

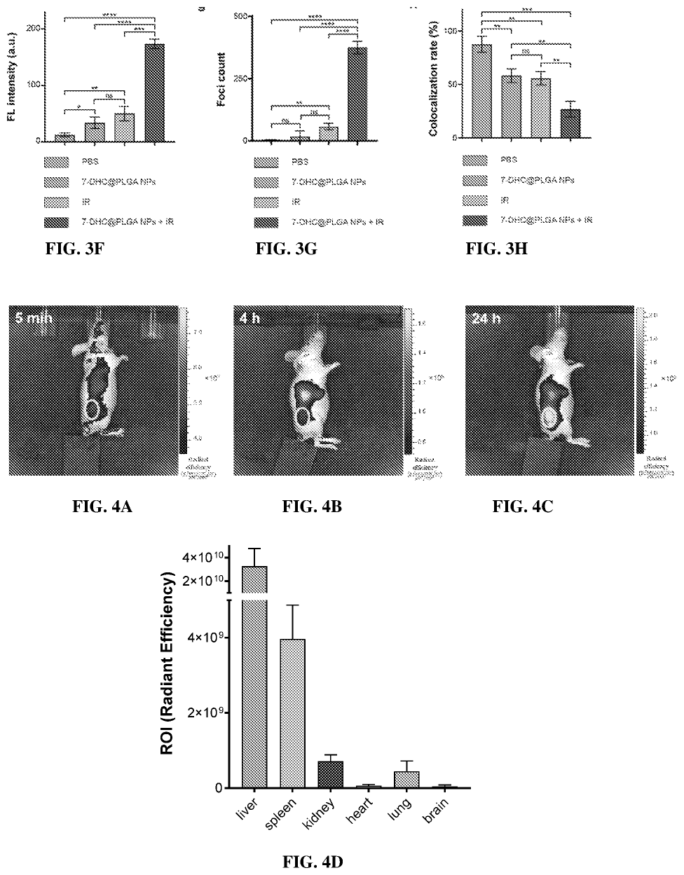

The resulting sterol radical can react with another 7-DHC molecule, leading to autoxidation. Alternatively, it reacts with other unsaturated lipids such as PUFAs, forming reactive peroxides (FIG. 3A). Either way, radical chain reactions are initiated and propagated, causing lipid peroxidation that amplifies radiation-induced damage. Notably, 7-DHC structurally resembles cholesterol and, similar to the latter, enriches in the lipid layers, including the plasma membrane and the mitochondrial membrane (Valencia, et al., Free Radic. Biol. Med., 41, 1704-1718 (2006)) This distribution is conductive to lipid membrane damage.

Both BODIPY Lipid Peroxidation (FIG. 3B) and TBARS assays (FIG. 3C) confirmed enhanced lipid peroxidation in CT26 cells treated with 7-DHC@PLGA NPs plus IR. In particular, TBARS assay found that the level of malondialdehyde (MDA), an aldehyde product of lipid peroxidation, was increased by 216.0% in cells treated with 7-DHC@PLGA NPs plus IR, compared to a 56.2% increase in cells treated with IR alone. Meanwhile, co-incubation with carnosine, an inhibitor of lipid peroxidation, (Xu, et al., J. Am. Chem. Soc., 131, 13037-13044 (2009)) mitigated toxicity induced by 7-DHC@PLGA NPs+IR (FIG. 2D). These results indicate a major role of lipid peroxidation in the radiosensitization effects of 7-DHC.

### Example 4: 7-DHC@PLGA NPs Augment Radiation-Induced Damage to Other Cellular Components

### Materials and Methods

### Reactive Oxygen Species Generation:

Specific Reactive Oxygen Species: Superoxide (O₂⁻) generation was tested using a Dihydroethidium assay kit (DHE, ThermoFisher™, Cat #D11347). Briefly, 7-DHC@PLGA NPs at a concentration of 5 μg/mL and 5 μM DHE were prepared in cell culture medium. 100 μL of 7-DHC@PLGA NP solution and 100 μL of sensor solution were added to a black 96-well-plate (Corning Costar, Cat #3614). The initial fluorescence was measured using a microplate reader (Synergy Mx, BioTeK) at excitation and emission wavelengths of 518/605 nm/nm respectively. Subsequently, the wells containing 7-DHC@PLGA NPs were irradiated with 5 Gy using a 50 kV X-ray generator and the fluorescence was measured again. The fluorescence was then compared to the initial fluorescence reading to evaluate the superoxide radical generation.

### Cell Uptake Study (Lysosome and Mitochondria Targeting) Using Fluorescence Imaging:

The 7-DHC@PLGA NP colocalization in the lysosome and mitochondria were tested using LysoTracker™ Green DND-26 (ThermoFisher, Cat #L7526) and MitoTracker™ Green FM (ThermoFisher, Cat #M7514), respectively. Briefly, 1×10⁵ CT26 cells were seeded on a 2-chamber glass slide (Nunc™ Lab-Tek™ II Chamber Slide™ System, ThermoFisher) and incubated for 24 h. Then 7-DHC@PLGA-Cy5 were added to the cells and incubated for an additional 24 h at 37° C. After the cells were washed with PBS 3 times, 90 nM LysoTracker or 148 nM MitoTracker was added to stain the lysosome or the mitochondria for 60 min or 4 h, respectively. The cells were fixed with 4% paraformaldehyde, and cell nuclei were stained with DAPI. The fluorescence image was taken using a Zeiss LSM 710 Confocal Microscope with 40× magnification.

### Mitochondrial Electric Membrane Potential (ΔΨm):

The change of mitochondrial membrane potential was measured by a JC-1 mitochondrial membrane potential detection kit (Biotium, Cat #30001). The JC-1 working solution was prepared by adding 10 μL of the concentrated dye to 1 mL of FBS free RPMI medium. 200 μL of cell culture medium containing Carbonyl Cyanide Chlorophenylhydrazone, (CCCP, positive control), DMSO (negative control), free 7-DHC (5 or 20 μg/mL), or 7-DHC@PLGA NPs (5 or 20 μg/mL) was incubated with cells for 4 h. The medium was removed and replaced with the JC-1 working solution to incubate for another 15 min. The fluorescence signal from the stained cells were detected using microplate reader (Synergy Mx, BioTeK; Green: ex/em
510/527 nm; Red: ex/em 585/590 nm). The green to red fluorescence ratios were calculated to evaluate mitochondrial depolarization.

### Cytochrome c Release:

Cytochrome c release induced by 7-DHC@PLGA NPs was tested using ApoTrack™ Cytochrome c Apoptosis ICC Antibody Kit (Abcam, Cat #ab110417). On the first day, 1×10⁵ CT26 cells were seeded in a 2-well chamber glass slide (Nunc™ Lab-Tek™ II Chamber Slide™ System, ThermoFisher, Cat #154461PK). The cells were incubated with PBS or 7-DHC@PLGA NPs (5 μg/mL) for 4 h. The cells were then irradiated with 5 Gy and incubated for 24 h. Then, ApoTrack™ Cytochrome c Apoptosis ICC Antibody was added following manufacture's protocol. Confocal images were taken at 40× magnification on a Zeiss LSM 710 Confocal Microscope to evaluate the fluorescence colocalization. The percent colocalization of the red and green fluorescent signals was quantified using ImageJ.

### Superoxide Dismutase (SOD) Assay:

CT26 cells at a density of 1.5×10⁵ cells per dish were seeded in a 6 well cell culture plate (Corning, Cat #3516) and incubated at 37° C. overnight. The cell culture medium was removed, and the cells were incubated for another 24 h with 1.5 mL of cell culture medium containing PBS, 7-DHC@PLGA NPs (5 μg/mL), IR only, or 7-DHC@PLGA NPs+IR (5 μg/mL). Each IR group was irradiated with 5 Gy and incubated for an additional 1 h. The cells were washed with PBS 3 times and collected using cell scraper followed by centrifugation (1200×g, 5 min). Next, the cells were dispersed in 1 mL PBS and lysed using a probe sonicator in an ice bath (30% amplitude, 5 min, 10 seconds on 10 seconds off). The supernatant was collected by centrifugation (1500×g, 5 min) and analyzed using the Superoxide Dismutase Assay Kit (Cayman Chemical, Cat #706002) following the manufacture's protocol. The absorbance at 450 nm was obtained using a microplate reader (Synergy Mx, BioTeK).

### γH2AX Assay:

The DNA damage was studied using anti-γH2AX (Alexa 647) antibody (Millipore Sigma, Cat #07-164-AF647). CT26 Cells (1×10⁶) were seeded in a 4-chamber glass slide (Nunc™ Lab-Tek™ II Chamber Slide™ System, ThermoFisher) and incubated overnight. Then, the cell culture medium was refreshed with 1.5 mL medium with 7-DHC@PLGA NPs (5 μg/mL), PBS, or 7-DHC@PLGA NPs+IR (5 μg/mL). After 4 h of incubation, X-ray radiation at 5 Gy was delivered to the IR only, and 7-DHC@PLGA NP+IR groups, and cells were incubated for another 1 h at 37° C. The cells were collected, fixed, permeabilized, and stained with anti-γH2AX antibody according to the protocol from the manufacturer. The presence of γH2AX protein was analyzed using a Zeiss LSM 710 Confocal Microscope and analyzed by ImageJ to evaluate and quantify the red fluorescence and the foci number for each group.

### Results

Next, the impact of 7-DHC@PLGA NPs on the mitochondria, which are rich in sterols and oxygen (Xu, et al, J. Am. Chem. Soc., 132, 2222-2232 (2010)), was examined. 7-DHC@PLGA NPs plus IR caused a 42.6% decrease in the mitochondrial membrane potential (Ψₘ), compared to a
28.7% decrease when IR alone was applied (FIG. 3D). It was hypothesized that depolarized mitochondria would disrupt the electron transport chain, leading to elevated ROS levels (Xu & Porter, J. Am. Chem. Soc., 136, 5443-5450 (2014)). Indeed, manganese superoxide dismutase (MnSOD) activity increased significantly in cells treated with 7-DHC@PLGA NPs plus IR (FIGS. 3E and 3F). Dihydroethidium (DHE) staining found that the cytosol superoxide level was significantly elevated, confirming an increased oxidative stress (FIG. 3F). This enhanced elevated oxidative stress translates to a broad destruction to other biomolecules in cells. For instance, γH2AX staining identified increased double-strand breaks in cells treated with 7-DHC@PLGA NPs plus IR (FIG. 3G). Furthermore, cytochrome c and mitochondrial complex Vα (inner mitochondrial membrane) double staining confirmed the release of cytochrome c into the cytosol (FIG. 3H), a trigger of the intrinsic apoptosis pathway. Overall, in addition to enhancing radiation-induced lipid peroxidation, 7-DHC@PLGA NPs also augment damages to other organelles, which culminate at inducing cell death.

### Example 5: 7-DHC@PLGA NPs Accumulate in Tumors Through the EPR Effect

### Materials and Methods

### In Vivo NIR Fluorescence:

100 μg DiR dye was added at the beginning of NP synthesis to make labelled μg 7-DHC@PLGA NPs. The tumor model was developed by subcutaneous injection of 2×10⁶ CT26 cells into the right flank of each animal. When the tumor size reached 300 mm³, each mouse was i.v. injected with 50 μg 7-DHC@PLGA NPs with DiR dye. Then, at 5 min, 4 h and 24 h p.i., the mice were anaesthetized and imaged using the IVIS Lumina II (Perkin Elmer). After 24 h the tumors and major organs such as liver, heart, lung, brain, kidney, and spleen were collected and imaged. The data was recorded by the radiant efficiency (p/sec/cm2/sr/μW/cm2).

### Results

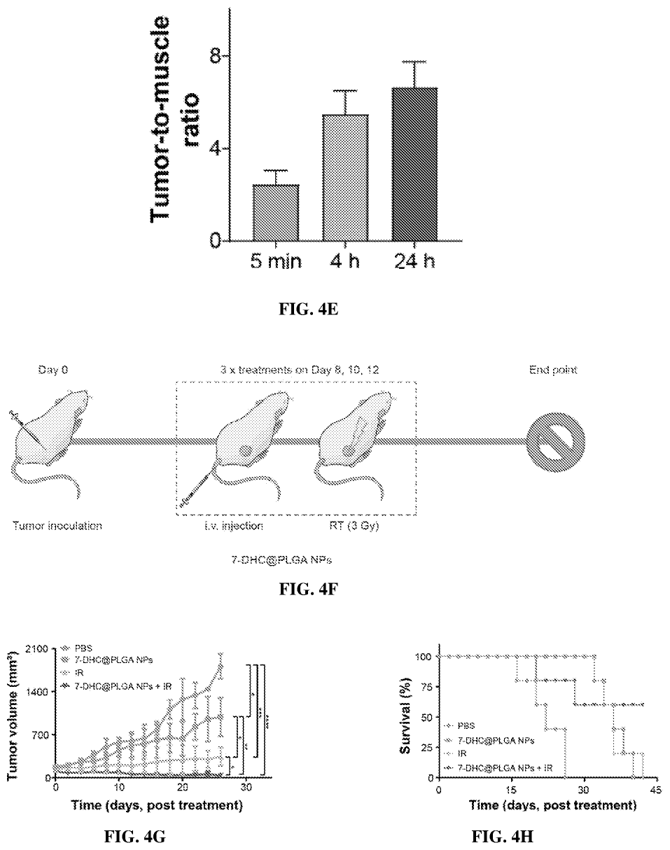

Prior to the therapy studies, the tumor targeting ability of 7-DHC@PLGA NPs was examined. 7-DHC@PLGA NPs were labelled with DiR, and the nanoparticles were intravenously (i.v.) administered into CT26 tumor bearing mice (n=3). The animals were subjected to whole-body fluorescence imaging on an IVIS system. 7-DHC@PLGA NPs were distributed throughout the body at early time points, but gradually accumulated in tumors (FIGS. 4A-4D). According to region of interest (ROI) analysis, the tumor-to-muscle ratio was increased by 4-fold at 24 h compared to 5 min (FIG. 4E). This is attributed to passive accumulation of 7-DHC@PLGA NPs in tumors through the EPR effect (Jin & Zhao, J. nanobiotechnology, 18, 1-17 (2020), Xu & Porter, Free Radic. Res., 49, 835-849 (2015)). The animals were euthanized after the 24-h imaging, and the major organs were harvested for ex vivo imaging. In addition to tumors, signals were found in the liver and spleen, which agrees with previous observations (Xu, et al., J. Lipid Res., 52, 1222-1233 (2011)).

### Example 6: 7-DHC@PLGA NPs Improve the Efficacy of Radiation Therapy

### Materials and Methods

### Therapy Studies:

All experiments were conducted in accordance with the guidelines from the University of Georgia Institutional Animal Care and Use Committee (IACUC). An in vivo therapy study was performed using 20 4-week old female Balb/c mice purchased from Charles River. The mice were cared for by following the Animal Use Protocol (AUP). The tumor model was developed by subcutaneous injection of 2×10⁶ CT26 cells into the right flank of each animal. When the tumor size reached 100 mm³, the mice were randomly divided into four groups (PBS, PBS+RT, and 7-DHC@PLGA NPs, 7-DHC@PLGA NPs+RT). The materials (10 mg/kg, 125 μL) were delivered by tail vein injection, and the tumors were irradiated with 5 Gy at 4 h after the injection, with the remainder of the body shielded by lead. The therapy was delivered every 48 h, for a total of 3 doses. The tumor size was measured every 2 days with calipers, and the tumor volume was calculated using the equation:

$$Tumor volume - \frac{tumor length \times tumor {width}^{2}}{2}$$

where tumor length≥tumor width. The mice were sacrificed when they reached a humane end point, including either length or width was >1.7 cm, the weight loss was more than 20%, or any tumor discharge was observed. The tumors and major organs such as liver, heart, lung, kidney, and spleen were collected for histological analysis using hematoxylin and eosin (H&E) staining. The tumors were also stained for Ki67, as well as using a TUNEL assay kit (Abcam, Cat #ab206386) to evaluate apoptotic cell death. Each stained tissue was examined under a digital microscope, and the most representative areas were captured and compared to see the difference between the groups.

### Results

Treatment efficacy of 7-DHC@PLGA NPs was evaluated in CT26 tumor bearing mice (FIGS. 4A-4C). On Day 8, 7-DHC@PLGA NPs (10 mg/kg 7-DHC) were i.v. injected, followed by IR radiation (320 kV, 3 Gy) applied to tumors (7-DHC@PLGA NPs+IR; n=10). The rest of the animal body was lead-shielded. Two more treatments were applied on Day 10 and
12 (FIG. 4F). For comparison, radiation only (IR, n=5), 7-DHC@PLGA NPs only (7-DHC@PLGA NPs, n=5), and carrier only (PBS, n=5) were tested. 7-DHC@PLGA NPs+IR was highly effective at slowing down tumor growth, showing an inhabitation rate of 98.4% on Day 26 relative to the PBS control group (FIG. 4G). By the end of the study on Day 43, 60% of the animals in the 7-DHC@PLGA NPs+IR group remained alive (FIG. 4H), half of which were tumor-free (FIG. 4G). As a comparison, all animals in other treatment groups, including those in the IR group, had either died or reached a humane endpoint by Day 43 (FIG. 4H). H&E staining found a significantly reduced density of cancer cells in tumors treated with 7-DHC@PLGA NPs+IR. An increased level of positive TUNEL staining and a reduced Ki67 staining was also observed, confirming effective radiosensitizing effects.

### Example 7: 7-DHC@PLGA NPs Induce No Systemic Toxicity or Hypercalcemia

### Materials and Methods

### Hematology and Blood Chemistry:

In a separate experiment, three Balb/c mice were intravenously injected with PBS or 7-DHC@PLGA NPs (5 mg/kg). A cardiac puncture method was used to collect blood samples. 250 μL of each of the blood samples were tested for a complete blood count to evaluate the total number of each type of blood cell. Remaining blood samples were used to evaluate liver function using the Alanine Aminotransferase (ALT) kit (Abcam, Cat #ab105134).

### Results

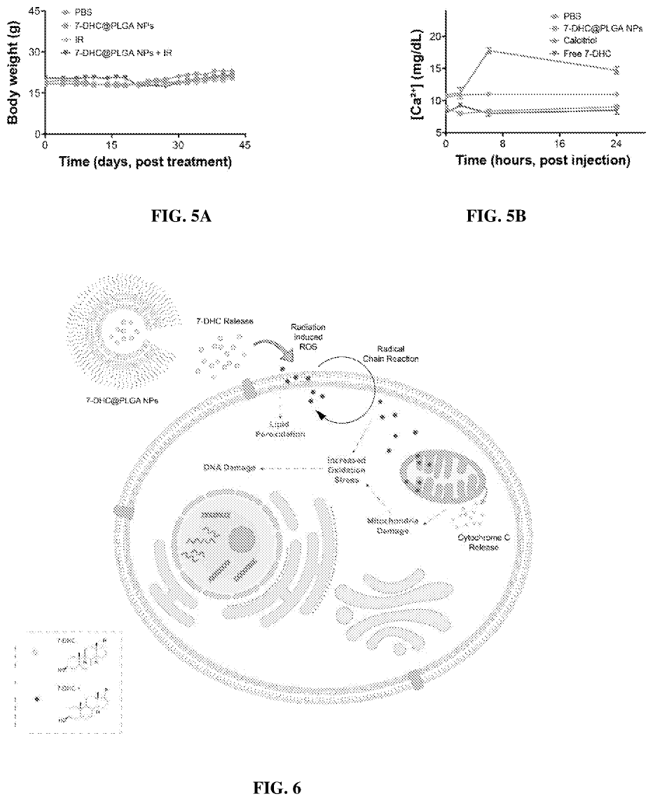

No actuate toxicity or animal body weight drop was observed during the therapy experiment (FIG. 5A). Post-mortem histology with normal tissues also detected no signs of side effects (FIG. 5B). To better understand the potential toxicity of 7-DHC@PLGA NPs, in a separate study, 7-DHC@PLGA NPs (10 mg/kg 7-DHC) was injected into healthy mice and tested blood samples for complete blood count (CBC) and biochemistry markers such as alanine aminotransferase (ALT). All indices were found to be within normal ranges (Table 3).

TABLE 3
CBC Analysis
CBC Analysis

| | | | Reference |
|---|---|---|---|
| Test | Result | Units | Interval |
| WBC | 8.1 | 10³/μL | 0.8-10.6 |
| Neu# | 1.59 | 10³/μL | 0.23-3.6 |
| Lym# | 5.99 | 10³/μL | 0.6-8.9 |
| Mon# | 0.32 | 10³/μL | 0.04-1.4 |
| Eos# | 0.13 | 10³/μL | 0.0-0.51 |
| Bas# | 0.02 | 10³/μL | 0.0-0.12 |
| Neu % | 19.7 | % | 6.5-50.0 |
| Lym % | 74.4 | % | 40-92 |
| Mon % | 4 | % | 0.9-18.0 |
| Eos % | 1.7 | % | 0.0-7.5 |
| Bas % | 0.2 | % | 0.0-1.5 |
| RBC | 11.18 | 10⁶/μL | 6.5-11.5 |
| Hgb | 17.7 | g/dl | 11-16.5 |
| Hct | 50.6 | % | 35-55 |
| MCV | 45.3 | fl | 41-55 |
| MCH | 15.8 | pg | 13-18 |
| MCHC | 35 | g/dl | 30-36 |
| RDW % | 13.2 | % | 12.0-19.0 |
| Platelet | 240 | 10³/μL | 400-1600 |
| MPV | 5.7 | Fl | 4-6.2 |
| ALT | 9.06 | U/mL | 15-84 |

In addition to being a cholesterol precursor, 7-DHC is also converted photochemically to vitamin D₃ in the skin (Xu, et al., J. Lipid Res., 52, 1810-1820 (2011)). The latter is transformed to calcidiol in the liver, and finally to the biologically active calcitriol in the proximal tubule of the kidneys (Xu, et al., J. Lipid Res., 52, 1810-1820 (2011)). This poses a potential risk of hypercalcemia as calcitriol is an important regulator for serum calcium. Indeed, injection of calcitriol at 200 μg/kg elevated the serum calcium to 17.5 mg/dl, which is almost twice the maximum tolerated level for mice (8.0-11.5 mg/dl) (Xu, et al., Biochim. Biophys. Acta, 1821, 877-883 (2012)). As a comparison, 7-DHC@PLGA NPs had a minimal impact on the calcium level. Overall, these studies support that 7-DHC@PLGA NPs are well tolerated by the animals.

7-DHC can efficiently promote radical chain reactions. While 7-DHC has low toxicity, it is highly susceptible to radical oxidation. In SLOS patients, the buildup of 7-DHC would cause an increase of the cellular ROS, forming a positive feedback loop that eventually leads to tissue damage. In the disclosed strategy, 7-DHC activation is triggered by radiation, which generates large amounts of ROS such as hydroxyl radical and superoxide in a short period of time, initiating radical chain reactions. This radiation-activatable property is unique among radiosensitizers. Together with nanoparticle delivery and safe metabolism of 7-DHC by DHCR7, the disclosed approach offers high tumor selectivity and minimal risks for long-term toxicity. Notably, 7-DHC and PUFA oxidation produce oxysterols and reactive aldehydes or ketones, (Windsor, et al., J. Lipid Res., 54, 2842-2850 (2013)) many of which are toxic compounds. These side products may also contribute to increased toxicity under radiation.

Interestingly, it is noted that many human colon cancer cells, including CT26, demonstrate a reduced expression of DHCR7 compared to normal tissues (Meljon, et al., Biochem. Pharmacol., 86, 43-55 (2013)). This property may add another dimension of selectivity to the approach; the drug tends to stay as 7-DHC in cancer cells rather than being metabolized to cholesterol. This may explain why 7-DHC@PLGA NPs alone caused moderate tumor suppression (FIG. 4E), but no systemic toxicity (FIG. 5B & Table 2). Meanwhile, nanoparticle pharmacokinetics and reductase conversion kinetics may affect the amount of 7-DHC inside cancer cells at the time of radiation, in turn influencing radiosensitizing efficiency.

### Example 8: 7-DHC@Low-Density Lipoprotein (LDL) Nanoparticles

### Materials and Methods

### 7-DHC@LDL and Cholesterol@LDL Particle Preparation

1. Prepared 6 mL of the solvent (2:1 v/v CHCl3:MeOH) with 1 mL of each following solution (in same solvent):
   6 μmol phosphatidyl choline (PC);
   4 μmol triolein (TO);
   2 μmol 7-DHC (or cholesterol for control).
2. Mixed in a round bottom flask, rotary evaporation at 60° C.
3. Add 1 mL of Tris buffer and keep stirring for an hour at 55-65° C.
4. Extruding the particle though a 100 nm membrane at 55-65° C.
5. Storage the particles at 4° C.
   See also FIG. 7L.

### Results

Next, low-density lipoprotein (LDL) was investigated as a carrier for 7-DHC. LDL is a naturally occurring transport for cholesterol, has a small size, long residence time, is biocompatible and biodegradable, favorably binds hydrophobic and amphiphilic drugs, and has a high loading rates.

### 7-DHC@LDL Nanoparticle Characterization

LDL nanoparticles containing 7-DHC were prepared. The particles were then characterized generally as discussed above with respect to 7-DHC@PLGA particles. Results yielded a dry weight of 4.5 mg/ml, average size of 105.7 nm (diameter), drug concentration of 0.467 mg/ml, and a loading rate of 60.69%. FIGS. 7A-7D compare the size distribution of 7-DHC@LDL (FIGS. 7A and 7B) and cholesterol@LDL (FIGS. 7C and 7D) particles before (FIGS. 7A and 7C) and after (FIGS. 7B and 7D) extrusion. FIGS. 7E and 7F show the zeta potential (surface charge) of 7-DHC- (FIG. 7E) and cholesterol- (FIG. 7F) loaded particles in water, PBS, and Tris Buffer at pH 8.

Drug release from 7-DHC@LDL NPs and cholesterol@LDL NPs was analyzed in buffer solutions with 5.5, 6.5, and 7.4 (FIGS. 7G and 7H respectively). 7-DHC@LDL particles, but not cholesterol@LDL particles, accelerated release at pH 5.5 corresponding to lysosomes or late endosomes, and reaching 100% by 72 hours.

Physical stability of 7-DHC@LDL NPs and cholesterol@LDL NPs were examined at 37° C. for one week. Changes in diameter and zeta potential are illustrated in FIGS. 7I-7K.

### Example 9: Targeting Moiety Enhances 7-DHC@LDL Particle Targeting

### Materials and Methods

A myristic acid linker was used to attach a neurotensin receptor (NTS) ligand/targeting signal ([MYRS]-Lys-Pro-(NMe-Arg)-Arg-Pro-Tyr-(Tle)-Leu-[COOH] (SEQ ID NO:7)) to 7-DHC@LDL NPs.

### Result

To investigate if a targeting moiety can increase delivery of 7-DHC@LDL NPs to target cells, an NTS peptide with Myristic acid linker was added to the particles are tested against H-1299 cells. H-1299 is a human non-small cell lung cancer cell line derived from the lymph node, having an NTS receptor. The size and zeta potential LDL NPs following the addition of the NTS targeting moiety are illustrated in FIGS. 8A-8C. The impact of the NTS targeting moiety on uptake of LDL particles in H-1299 cells treated with two different concentration is illustrated in FIG. 8D. Results show that at both concentrations, NTS targeting increases cellular update of 7-DHC@LDL NPs into H-1299 cells.

### Example 10: 7-DHC@LDL NPs Sensitize Cells to Ionizing Radiation

### Materials and Methods

Cells were treated with 7-DHC@LDL or control cholesterol@LDL NPs, with or without NTS targeting moiety, ±5 Gy radiation, 4 hours of update, and 48 hours of incubation. Viability (MTT), γH2AX expression, ROS, SOD, and lipid peroxidation (4-HNE, MDA) were analyzed.

### Results

To investigate the radiosensitizing ability of 7-DHC@LDL NPs, cells were treated 7-DHC@LDL or control cholesterol@LDL NPs, with or without NTS targeting moiety, in the presence or absence of ionizing radiation, and analyzed for viability (MTT assay), γH2AX, ROS, SOD, and lipid peroxidation (4-HNE, MDA). Aripiprazole inhibitor (“In”) was used as a positive control.

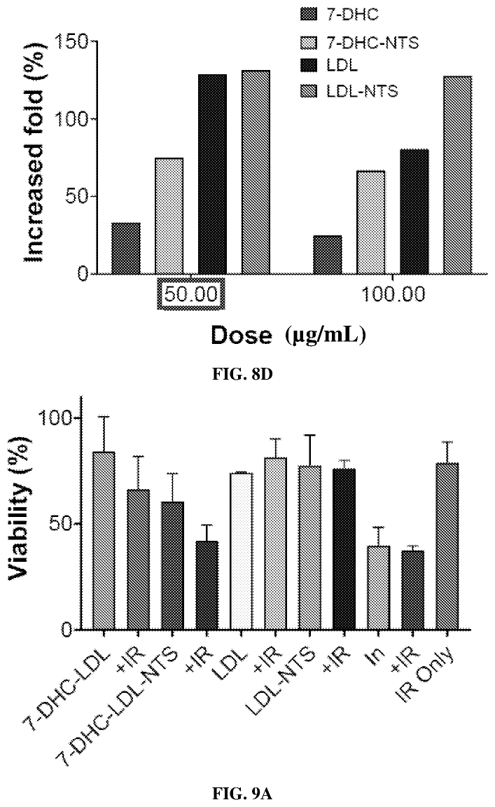

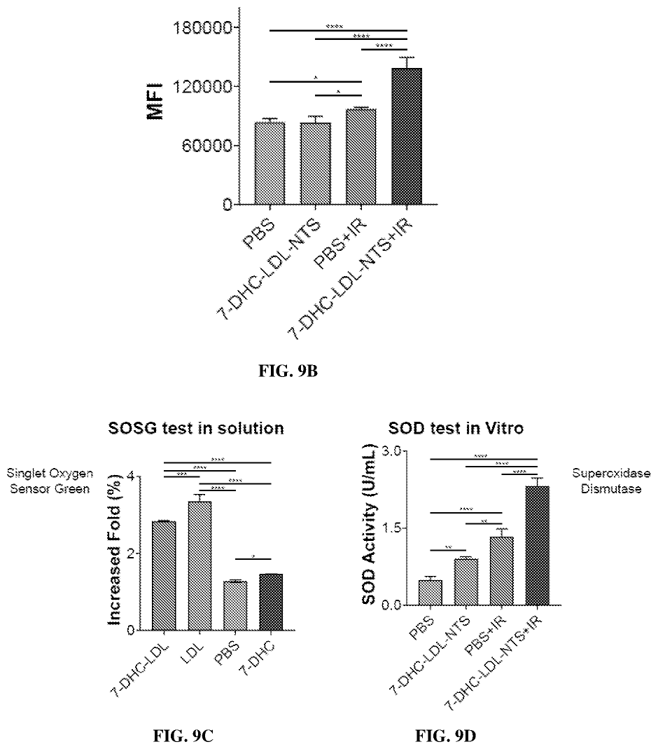

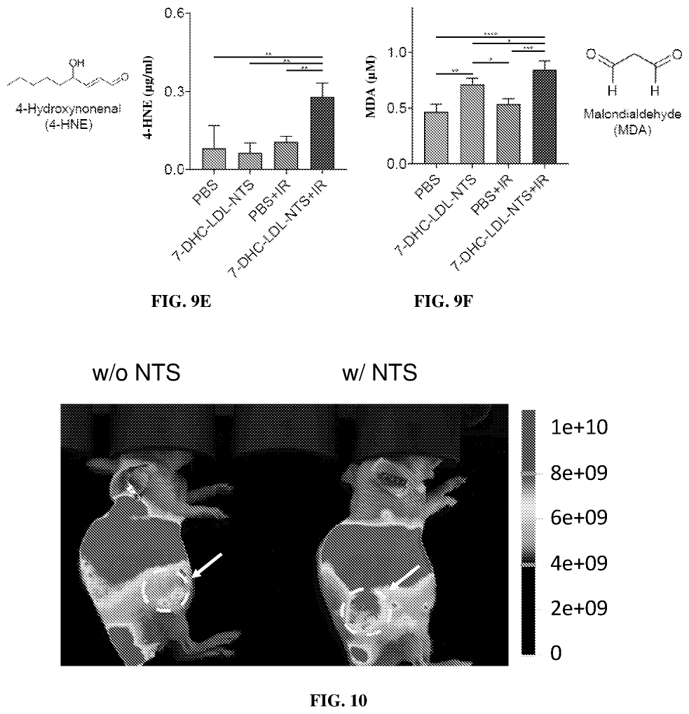

Results are presented in FIGS. 9A-9F. Ionizing radiation further reduces the viability of cells treated with 7-DHC@LDL NPs with and without targeting moiety, where 7-DHC@LDL NPs with NTS targeting moiety generated the greatest reduction in cell viability of all experimental groups and was comparable to the Aripiprazole inhibitor-treated positive control (FIG. 9A).

7-DHC@LDL-NTS NPs with ionizing radiation increased expression of γH2AX relative to 7-DHC@LDL-NTS NPs or radiation alone (FIG. 9B).

7-DHC@LDL increased ROS according to a Singlet Oxygen Sensor Green (SOSG) test in solution (FIG. 9C). 7-DHC@LDL+NTS both without, but particularly with, ionizing radiation increased ROS as evident by an increase is Superoxidase Dismutase (SOD) activity (U/ml) in vitro (FIG. 9D).

7-DHC@LDL-NTS, particularly with ionizing radiation, increased lipid peroxidation as evident by measuring 4-hydroxynonenal (4-HNE) (FIG. 9E) and malondialdehyde (MDA) (FIG. 9F).

Example 7-9 show successful assembly of 7-DHC@LDL NPs with excellent size, stable physical structure, and suitable drug release. A targeting moiety that increases cell update rate can be added. 42% cell toxicity toward targeted H-1299 cells were achieved under 5 Gy irradiation, while producing ROS and inducing lipid peroxidation on the cell membrane.

Collectively these results show that 7-DHC@LDL NPs with or without a targeting moiety can serves as a radiosensitizer in the presence of ionizing radiation. It is believed that this occurs generally through the mechanism described above for 7-DHC@PLGA NPs. 7-DHC is released from the NPs and accumulated in cancer cells' plasma and mitochondrial membrane. Under radiation, 7-DHC triggers and propagates radical chain reactions that cause lipid peroxidation and mitochondria damage, exacerbating oxidative stress in cells. Meanwhile, lipid peroxidation also produces toxic oxysterols and aldehydes that react with DNA and other biomolecules. All these events culminate at inducing cell death.

### Example 11: 7-DHC Liposomal Nanoparticles Target Tumors

### Materials and Methods

7-DHC nanoparticles, specifically 7-DHC-encapsulated liposomes, were prepared and assessed in H1299 tumor bearing mice.

Briefly, phosphatidyl choline, triolein, and 7-dehydrocholesterol (7-DHC) at a 3:2:1 molar ratio was dissolved in a CHCl₃/MeOH solvent. After removing the solvent, tris buffer (pH=8.0) was added to the flask to reconstitute the nanoparticles, which then underwent extrusion through a 100-nm filter. The 7-DHC loading capacity (LC %) is 10.4% and the encapsulation efficiency (EE %) is 60.7%.

The 7-DHC nanoparticles were coupled with or without a tumor targeting ligand (NTS, which binds to NTSR1 or neurotensin receptor 1 that is overexpressed in many types of tumors) and were labeled with a near-infrared dye.

To investigate the radiosensitizing effects of 7-DHC-liposomes in H1299 tumor bearing mice, the nanoparticles were coupled with NTS for tumor targeting, and were intravenously administered at 2.3 mg 7-DHC/kg. Radiation (RT, 5 Gy) was applied to tumors 24 hours post-particle injection. During radiation, the rest of the animal body was lead-shielded. A total of three treatments were performed two days apart.

Whole-body fluorescence images were taken on a small animal imaging scanner.

### Results

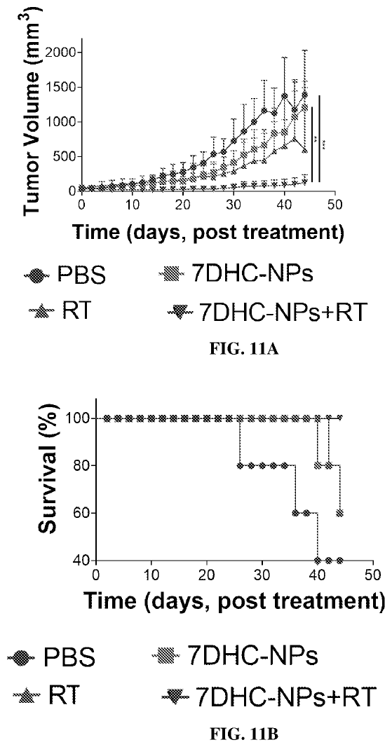

To investigate the tumor targeting ability of 7-DHC liposome NPs, H2199 mice were treated 7-DHC liposome NPS, with or without NTS targeting moiety. FIG. 10 are 24-hour images showing the results. Following intravenous (i.v.) injection, the 7-DHC-liposomes, in particular, those coupled with NTS, were able to accumulate in the tumors (as indicated by the arrow). As shown in FIG. 10, 7-DHC-liposomes also accumulate in the liver and spleen post-injection, which is common among nanoparticles.

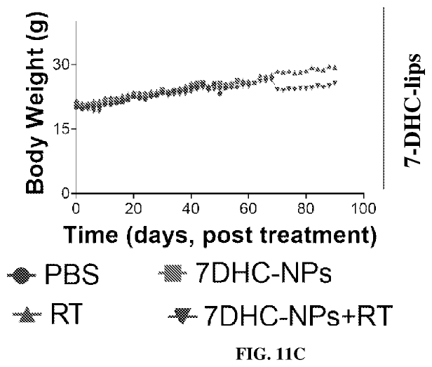

As shown in FIGS. 11A-11C, the combination of 7-DHC-liposomes and RT led to significant tumor inhibition compared to radiation (RT) alone. The tumor growth inhibition rate was 90.9% on Day 44, compared to 56.9% for RT alone. By the end of the study on Day 44, all animals in the 7-DHC-Liposomes plus RT group remained alive (FIG. 11B), and 40% of them were tumor-free.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A method of treating a subject in need thereof comprising administering the subject a pharmaceutical composition comprising
an effective amount of 7-dehydrocholesterol (7-DHC) or analog or derivative thereof, and
one or more doses of radiation therapy, optionally non-ultraviolet radiation therapy.

2. The method of claim 1, wherein the subject has cancer.

3. A method of treating cancer comprising administering a subject with cancer a pharmaceutical composition comprising
an effective amount of 7-dehydrocholesterol (7-DHC) or analog or derivative thereof, and
one or more doses of radiation therapy, optionally non-ultraviolet radiation therapy.

4. The method of any one of claims 1-3, wherein the radiation therapy comprises X-rays, gamma rays, protons, or neutrons; and/or
wherein the radiation is photodynamic therapy or ionizing radiation therapy;
optionally wherein the radiation therapy is administered in an effective amount to induce the chemical reaction according to Scheme 1 with 7-DHC or the corresponding chemical reaction in the analog or derivative thereof.

5. The method of any one of claims 1-4, wherein the 7-DHC or analog or derivative thereof is encapsulated or incorporated into or otherwise forms part of nanoparticles.

6. The method of claim 5, wherein the nanoparticles are polymeric nanoparticles, lipoprotein-like particles, liposomes, inorganic nanoparticles, or a combination thereof.

7. The method of claim 6, wherein the nanoparticles are polymeric nanoparticles comprising one or more amphiphilic, hydrophobic, and/or hydrophilic polymers.

8. The method of claim 7, wherein the nanoparticle comprises one or more hydrophobic polymers.

9. The method of claim 8, wherein one or more of hydrophobic polymers is polyester.

10. The method of claim 9, wherein the polyester or polyesters are selected from poly(lactic acid-co-glycolic acid)s, poly(lactic acid), poly(glycolic acid).

11. The method of any one of claims 5-10, wherein the nanoparticles comprise poly(lactic acid-co-glycolic acid) (PLGA).

12. The method of any one of claims 5-10, wherein the nanoparticles are lipoprotein or lipoprotein-like nanoparticles.

13. The method of claim 12, wherein the nanoparticles are low-density lipoprotein (LDL) or LDL-like nanoparticles.

14. The method of claims 12 or 13, wherein the nanoparticles comprise one or more triglyceride(s) and one or more phospholipid(s).

15. The method of claim 14, wherein the triglyceride(s) comprises oleic acid (OA) and/or triolein (TO).

16. The method of claims 14 and 15, wherein the phospholipid(s) comprise phosphatidyl choline (PC).

17. The method of any one of claims 12-16, wherein the lipoprotein or lipoprotein-like nanoparticles are synthetic.

18. The method of any one of claims 12-16, wherein the lipoprotein or lipoprotein-like nanoparticles is natural lipoprotein isolated from a human subject, optionally wherein the lipoprotein is natural LDL.

19. The method of any one of claims 12-18, wherein the lipoprotein or lipoprotein-like nanoparticles are free from protein.

20. The method of any one of claims 5-19, wherein the nanoparticles have a size suitable for delivery of the 7-DHC to tumor microenvironments by enhanced permeability and retention.

21. The method of any one of claims 5-19, wherein the nanoparticle have a size of about 10 nm to about 300 nm, or about 50 nm to about 150 nm.

22. The method of any one of claims 5-19, wherein the nanoparticles further comprise a targeting agent coupled thereto.

23. The method of claim 22, wherein the targeting agent targets NTSR1.

24. The method of claim 23, wherein the targeting agent is an agonist or antagonist for NTSR1.

25. The method of any one of claims 22-24, wherein the targeting agent is NTS or a variant thereof.

26. The method of claim 25, wherein targeting agent is NTSₘᵤₜ.

27. The method of any one of claims 24-26, wherein the targeting agent is SR142948A or NTS_{20.8}.

28. The method of any one of claims 22-27, wherein the target agent comprising or consisting of the sequence of any one of SEQ ID NOS:1-7.

29. The method of any one of claims 1-28, wherein the composition enhances the treatment of the cancer compared to administration of the radiation alone.

30. The method of any one of claims 1-29, wherein the cancer is a radiosensitive cancer.

31. The method of any one of claims 1-29, wherein the cancer is a radioresistant cancer.

32. The method of any one of claims 1-31, wherein the cancer a vascular, bone, muscle, bladder, brain, breast, cervical, cob-rectal, esophageal, kidney, liver, lung, nasopharyngeal, pancreatic, prostate, skin, stomach, uterine, or germ cell.

33. The method of any one of claims 1-32, wherein the cancer is an epithelial cancer.

34. The method of any one of claims 1-32, wherein the cancer is a non-small cell lung cancer (NSCLC).

35. The method of any one of claims 1-34, wherein the same dose of radiation is more effective than when administered in the absence of the pharmaceutical composition, a lower dose of radiation has the same effectiveness as a higher dose when administered in the absence of the pharmaceutical composition, or a combination thereof.

36. The method any one of claims 1-35, wherein a dose of radiation is administered after administration of the pharmaceutical composition.

37. The method of any one of claims 1-36, wherein the dose of radiation is administered 1 to 48 hours, or 1 to 24 hours, or 1 to 12 hours, or 1 to 6 hours, or 2 to 6 hours, or 1, 2, 3, 4, or 5 hours after administration of the pharmaceutical composition.

38. The method of any one of claims 1-37, comprising 1, 2, 3, 4, 5, 6, 7, 8, 9, 10, or more rounds of administration of the pharmaceutical composition followed by administration of the dose of radiation, optionally wherein the dose of radiation is administered after one, some, or all of the rounds of administration of the pharmaceutical composition.

39. The method of any one of claims 1-38, wherein the 7-DHC or analog or derivative thereof is 7-DHC.

40. The method of any one of claims 1-38, wherein the 7-DHC or analog or derivative thereof is an analog or derivative thereof is 7-DHC.

41. The method of claim 40, wherein the analog or derivative thereof is a compound of any of: 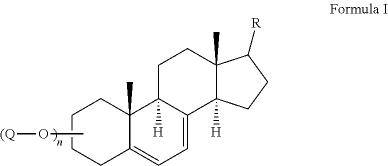
wherein n is an integer value selected from 1, 2, 3, or 4;
wherein R is a linear or branched alkyl or heteroalkyl group, alkenyl or heteroalkenyl, or alkynyl or heteroalkynyl group, where each may be optionally substituted by one or more substituents; and
wherein Q is a hydrogen, a linear or branched alkyl or heteroalkyl group, alkenyl or heteroalkenyl, or alkynyl or heteroalkynyl group, a cycloalkyl group, a heterocycloalkyl, an aryl, a heteroaryl group, acyl group, a sulfonate group, or a siloxy group, where each may be optionally substituted with one or more substituents;
or is a salt thereof; 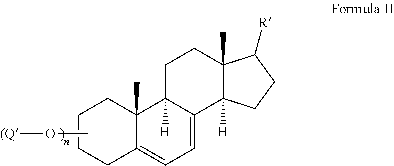
wherein n is an integer value selected from 1, 2, 3, or 4;
wherein R′ is a linear or branched alkyl or heteroalkyl group, alkenyl or heteroalkenyl, or alkynyl or heteroalkynyl group, where each may be optionally substituted by one or more substituents; and
wherein Q′ is a hydrophilic polymer or a hydrophilic saccharide; or is a salt thereof: 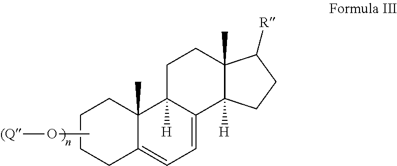
wherein n is an integer value selected from 1, 2, 3, or 4;
wherein R″ is a linear or branched alkyl or heteroalkyl group, alkenyl or heteroalkenyl, or alkynyl or heteroalkynyl group, where each may be optionally substituted by one or more substituents; and
wherein Q″ is a therapeutic agent;
or is a salt thereof; 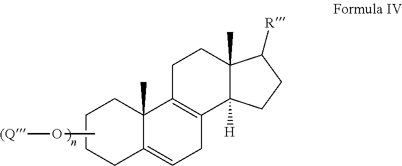
wherein n is an integer value selected from 1, 2, 3, or 4;
wherein R″′ is a linear or branched alkyl or heteroalkyl group, alkenyl or heteroalkenyl, or alkynyl or heteroalkynyl group, where each may be optionally substituted by one or more substituents; and
wherein Q″′ is a hydrogen, a linear or branched alkyl or heteroalkyl group, alkenyl or heteroalkenyl, or alkynyl or heteroalkynyl group, a cycloalkyl group, a heterocycloalkyl, an aryl, a heteroaryl group, acyl group, a sulfonate group, or a siloxy group, where each may be optionally substituted with one or more substituents;
or is a salt thereof; 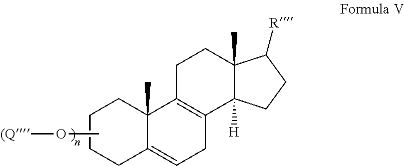
wherein n is an integer value selected from 1, 2, 3, or 4;
wherein R″″ is a linear or branched alkyl or heteroalkyl group, alkenyl or heteroalkenyl, or alkynyl or heteroalkynyl group, where each may be optionally substituted by one or more substituents; and
wherein Q″″ is a hydrophilic polymer or a hydrophilic saccharide; or is a salt thereof; or 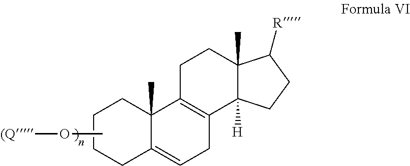
wherein n is an integer value selected from 1, 2, 3, or 4;
wherein R″″′ is a linear or branched alkyl or heteroalkyl group, alkenyl or heteroalkenyl, or alkynyl or heteroalkynyl group, where each may be optionally substituted by one or more substituents; and
wherein Q″″′ is a therapeutic agent;
or is a salt thereof.

42. The method of claims 40 and 41 wherein a plurality of the analog or derivative thereof can self-assemble into nanoparticles.

43. A compound of any of Formulae I-VI.

44. A nanoparticle comprising a compound of any of Formulae I-VI.

45. The compound or nanoparticle of claims 43 or 44 further comprising a therapeutic agent conjugated thereto.

46. The compound or nanoparticle of claim 45, wherein the therapeutic agent is a small molecule chemotherapeutic agent.

47. The nanoparticle of any one of claims 44-46 formed by self-assembly of a plurality of the compounds of Formulae II or V alone or in combination with one or more other polymers, lipids, proteins, or saccharides.

48. A pharmaceutical composition comprising the compound or nanoparticle of any one of claims 43-47.

49. The pharmaceutical composition of any one of claims 1-42.

50. The pharmaceutical composition of claim 49 comprising the nanoparticles according to any of claims 3-28.


SEQUENCE LISTING (7 sequences)

SEQ ID NO: 1 - protein, 9 aa, synthetic construct
XXCNGRCXX

SEQ ID NO: 2 - protein, 11 aa, synthetic construct
CXXXNGRXXXC

SEQ ID NO: 3 - protein, 11 aa, synthetic construct
CNGRCXXXXXX

SEQ ID NO: 4 - protein, 7 aa, synthetic construct
CRGRRST

SEQ ID NO: 5 - protein, 13 aa, Homo sapiens
QLYENKPRRPYIL

SEQ ID NO: 6 - protein, 7 aa, synthetic construct
PRRPYIL

SEQ ID NO: 7 - protein, 8 aa, synthetic construct
  MOD_RES 1: note = Myristic acid linker
  MOD_RES 3: note = N-methylated Arginine
  MOD_RES 7: note = Tert-Leucine
KPXRPYXL